(19) United States
(12) Patent Application Publication
(10) Pub. No.: US 2024/0156366 A1
(43) Pub. Date: May 16, 2024

(54) GASTROINTESTINAL TREATMENT SYSTEM INCLUDING A PERSONALIZED VIBRATING CAPSULE, AND METHOD OF USE THEREOF

(71) Applicant: Vibrant Ltd., Yokneam (IL)

(72) Inventors: Lior BEN-TSUR, Netanya (IL); Shai MOLNAR, Shorashim (IL); Roni SHABAT, Kibbutz Yizra'el (IL)

(21) Appl. No.: 18/383,903

(22) Filed: Oct. 26, 2023

Related U.S. Application Data
(63) Continuation-in-part of application No. 17/961,616, filed on Oct. 7, 2022.
Application No. 17/961,616 is a continuation of application No. 17/461,053, filed on Aug. 30, 2021, now Pat. No. 11,504,024.
Application No. 17/461,053 is a continuation-in-part of application No. 17/038,226, filed on Sep. 30, 2020.
Application No. 17/038,226 is a continuation-in-part of application No. PCT/IB2019/052529, filed on Mar. 28, 2019.
Application No. PCT/IB2019/052529 is a continuation-in-part of application No. 17/233,587, filed on Apr. 19, 2021.
Application No. 17/233,587 is a continuation of application No. 16/357,570, filed on Mar. 19, 2019, now Pat. No. 11,478,401.
Application No. 16/357,570 is a continuation-in-part of application No. PCT/IB2017/055565, filed on Sep. 14, 2017.

(30) Foreign Application Priority Data
Sep. 21, 2016 (GB) 1616044.2
Mar. 30, 2018 (GB) 1805325.6
May 31, 2018 (GB) 1808859.1

(51) Int. Cl. A61B 5/07; A61B 5/00; A61H 23/02

(52) U.S. Cl. CPC A61B 5/073; A61B 5/42; A61B 5/4836; A61H 23/02; A61H 23/0263; A61B 2560/0214; A61B 2562/0219; A61B 2562/0247; A61B 2562/0271; A61B 2562/162; A61H 2201/1207; A61H 2205/083

(57) ABSTRACT

A gastrointestinal treatment system including a gastrointestinal capsule adapted to treat a subject following ingestion of the gastrointestinal capsule. The gastrointestinal capsule includes: (a) a housing; (b) a vibrating agitator, powered by the battery, the vibrating agitator adapted such that, in a first vibrating mode of operation, the housing exerts vibrations on an environment surrounding the capsule; (c) a power supply disposed within the housing and adapted to power the vibrating agitator; and (d) a controller adapted, in response to receipt of an activation input, to activate the vibrating agitator to operate in the first vibrating mode of operation at a user-specific time of day.

### RELATED APPLICATIONS

The present application is a continuation in part of U.S. patent application Ser. No. 17/233,587, filed on Apr. 19, 2021. U.S. patent application Ser. No. 17/233,587 is a continuation of U.S. patent application Ser. No. 16/357,570, filed on Mar. 19, 2019. U.S. patent application Ser. No. 16/357,570 is a continuation in part of PCT Patent Application No. PCT/IB2017/055565, filed on Sep. 14, 2017. PCT Patent Application No. PCT/IB2017/055565 gains priority from GB Patent Application No. 1616044.2, filed on Sep. 21, 2016, and from Chinese Patent Application No. 2017210463282, filed on Aug. 21, 2017.

The present application is also a continuation in part of U.S. patent application Ser. No. 17/961,616 filed on Oct. 7, 2022. U.S. patent application Ser. No. 17/961,616 is a continuation of U.S. patent application Ser. No. 17/461,053, filed on Aug. 30, 2021. U.S. patent application Ser. No. 17/461,053 is a continuation in part of U.S. patent application Ser. No. 17/038,226 filed Sep. 30, 2020. U.S. patent application Ser. No. 17/038,226 is a continuation in part of PCT Patent Application No. PCT/IB2019/052529, filed on Mar. 28, 2019. PCT Patent Application No. PCT/IB2019/052529, filed on Mar. 28, 2019 gains priority from GB Patent Application No. 1805325.6, filed on Mar. 30, 2018, and from GB Patent Application No. 1808859.1, filed on May 31, 2018.

U.S. patent application Ser. Nos. 17/233,587; 16/357,570; 17/961,616; 17/461,053; and Ser. No. 17/038,226: PCT Patent Application Nos. PCT/IB 2017/055565 and PCT/IB2019/052529; GB Patent Application Nos. 1616044.2; 1805325.6; and 1808859.1; and Chinese Patent Application No. 2017210463282 are all incorporated by reference as if fully set forth herein.

### FIELD OF THE INVENTION

The present invention relates in general to gastrointestinal treatment systems including one or more vibrating capsules, and to methods of use thereof. The present invention further relates to methods and systems for personalizing a treatment protocol of such vibrating capsules, such that the one or more capsules vibrate, or are adapted to vibrate, at one or more specific and personalized times of day. The present invention further relates in general to a method and a system for mitigating at least one effect of jetlag, and specifically to a method for mitigating jetlag using a gastrointestinal treatment system including one or more vibrating capsules.

### SUMMARY OF THE INVENTION

In accordance with an embodiment of the present invention, there is provided a gastrointestinal treatment system for treatment of a disorder in a gastrointestinal tract of a subject, the system including an input source providing input relating to a characteristic of a circadian cycle of the subject. The system further includes a control unit, functionally associated with the input source, the control unit adapted to determine a user-specific time of day based on the input relating to the characteristic of the circadian cycle of the subject. The system further includes a gastrointestinal capsule for vibrating in the gastrointestinal tract of the subject following ingestion of the gastrointestinal capsule. The gastrointestinal capsule includes a housing, a vibrating agitator adapted such that, in a first vibrating mode of operation, the housing exerts vibrations on an environment surrounding the capsule, a power supply disposed within the housing and adapted to power the vibrating agitator, and a controller adapted, in response to receipt of an activation input from the control unit, to activate the vibrating agitator to operate in the first vibrating mode of operation at the user-specific time of day.

In some embodiments, the input source includes a user interface, adapted to receive input from the subject or from a caretaker of the subject.

In some embodiments, the input source includes a sensor, adapted to sense a characteristic relating to the circadian cycle of the subject and to provide the input.

In some embodiments, the input includes input relating to a sleep time or sleep schedule of the subject.

In some embodiments, the input includes input relating to a meal time or meal schedule of the subject.

In some embodiments, the input includes input relating to a time of defecation of the subject.

In some embodiments, the input includes input relating to a vitality schedule of the subject.

In some embodiments, the user-specific time of day is a user-specific mealtime.

In some embodiments, the gastrointestinal treatment system further includes a computer readable memory adapted to store a subject profile for the subject, the subject profile including subject data received as the input.

In some embodiments, the control unit is adapted to provide the user-specific time of day, to the gastrointestinal capsule, as part of the activation input.

In some embodiments, the control unit is adapted to incorporate the user-specific time of day into a vibration protocol and to program the gastrointestinal capsule with the vibration protocol.

In accordance with an embodiment of the present invention, there is provided a method of treating a gastrointestinal condition of a subject, the method including:
(a) receiving input relating to a characteristic of a circadian cycle of the subject;
(b) determining a user-specific time of day based on the input relating to the characteristic of the circadian cycle of the subject;
(c) programming a gastrointestinal capsule being adapted to transit a gastrointestinal tract of the subject, with the user-specific time of day, the gastrointestinal capsule having:
   (1) a housing;
   (2) a vibrating agitator, the vibrating agitation mechanism adapted such that, in a first vibrating mode of operation, the housing exerts vibrations on an environment surrounding the gastrointestinal capsule;
   (3) a power supply disposed within the housing and adapted to power the vibrating agitator; and
   (4) a controller adapted to activate the vibrating agitator to operate in the first vibrating mode of operation;
(d) following ingestion of the gastrointestinal capsule by the subject, and when the gastrointestinal capsule is in an operative state, activating the vibrating agitator to operate in the first vibrating mode of operation the user-specific time of day.

In some embodiments, receiving input includes receiving input relating to a sleep schedule of the subject.

In some embodiments, receiving input includes receiving input relating to a meal schedule of the subject.

In some embodiments, receiving input includes receiving input relating to a time of defecation of the subject.

In some embodiments, receiving input includes receiving input relating to a vitality schedule of the subject.

In some embodiments, receiving input includes receiving the input from the subject or from a caregiver of the subject.

In some embodiments, receiving input includes receiving the input from a sensor.

In some embodiments, the method further includes storing the input in a subject-profile associated with the subject.

In some embodiments, programming includes providing the user-specific time of day to the gastrointestinal capsule as part of an activation input. In accordance with an embodiment of the present invention, there is provided a gastrointestinal treatment system including a gastrointestinal capsule adapted to treat a gastrointestinal tract of a subject following ingestion of the gastrointestinal capsule, the gastrointestinal capsule including:
(a) a housing;
(b) a vibrating agitator adapted such that, in a first vibrating mode of operation, the housing exerts vibrations on an environment surrounding the capsule;
(c) a power supply disposed within the housing and adapted to power the vibrating agitator; and
(d) a controller adapted, in response to receipt of an activation input, to activate the vibrating agitator to operate in the first vibrating mode of operation at at least one predetermined time of day.

In accordance with another embodiment of the present invention, there is provided a gastrointestinal treatment system including a gastrointestinal capsule adapted to treat a gastrointestinal tract of a subject following ingestion of the gastrointestinal capsule, the gastrointestinal capsule including:
(a) a housing;
(b) a vibrating agitator adapted such that, in a first vibrating mode of operation, the housing exerts vibrations on an environment surrounding the capsule;
(c) a power supply disposed within the housing and adapted to power the vibrating agitator; and
(d) a controller adapted, when the capsule is in an operative state, to activate the vibrating agitator to operate in the first vibrating mode of operation at at least one predetermined time of day.

In some embodiments, the capsule is adapted to be in the operative state following receipt of an activation input, which transitions the capsule from an inoperative state to an operative state.

In some embodiments, the subject is travelling from an origin location to a destination location, and the system is adapted to mitigate jetlag of the subject.

In some such embodiments, the controller is adapted to activate the vibrating agitator to operate in the first vibrating mode of operation at at least one predetermined time of day according to a time zone of the origin location.

In some such embodiments, the controller is adapted to activate the vibrating agitator to operate in the first vibrating mode of operation at at least one predetermined time of day according to a time zone of the destination location.

In some such embodiments, the controller is adapted to activate the vibrating agitator to operate in the first vibrating mode of operation at a first time of day of the at least one predetermined time of day according to a time zone of the origin location and at a second time of day of the at least one predetermined time of day according to a time zone of the destination location.

In some embodiments, the predetermined time of day is selected according to a circadian cycle of the subject.

In some embodiments, the predetermined time of day is selected according to a gastric pH profile of the subject.

In some embodiments, the capsule includes at least one timing mechanism, and is devoid of sensors for sensing an environment thereof.

In some embodiments, the at least one timing mechanism comprises a timer.

In some embodiments, the at least one timing mechanism comprises a clock. In some embodiments, the at least one timing mechanism is associated with a positioning system such as GPS.

In some embodiments, the controller is adapted, in response to the activation input, to wait a predetermined delay time, and following the predetermined delay time, at a time coinciding with the at least one predetermined time of day, to activate the vibrating agitator to operate in the first vibration mode of operation.

In some embodiments, the capsule further includes at least one sensor adapted to provide the activation input.

In some embodiments, the at least one sensor includes an illumination sensor adapted to provide the activation input upon identification of transition of the capsule from an illuminated environment to a dark environment.

In some embodiments, the at least one sensor includes a pressure sensor adapted to provide the activation input upon identification of pressure applied to the capsule, which pressure is indicative of the capsule moving through a pharynx of the subject.

In some embodiments, the at least one sensor includes a temperature sensor adapted to provide the activation input upon identification of transition of the capsule from an area with ambient temperature to an area with a human body temperature.

In some embodiments, the at least one sensor includes an accelerometer adapted to provide the activation input upon identification of an activation motion carried out by a user of the gastrointestinal capsule.

In some embodiments, the at least one sensor includes a moisture sensor adapted to provide the activation input upon identification of transition of the capsule from a dry environment to a humid environment.

In some embodiments, the capsule further includes a timing mechanism. In some embodiments, in response to the activation input, the controller is adapted to activate operation of the timing mechanism to track a time of day so as to identify the at least one predetermined time of day for activation of the vibration agitator. In some embodiments, in response to the activation input, the controller is adapted to activate operation of the timing mechanism to wait a predetermined duration prior to activation of vibration agitator, such that activation of the vibration agitator occurs at the at least one predetermined time of day.

In some embodiments, the system further includes a control unit, adapted to provide the activation input to the controller of the gastrointestinal capsule.

In some embodiments, the control unit is adapted to provide the activation input following ingestion of the gastrointestinal capsule by the subject. In other embodiments, the control unit is adapted to provide the activation input prior to ingestion of the gastrointestinal capsule by the subject.

In some embodiments, the control unit is adapted to provide to the controller a current time of day, and the controller is adapted to compute a delay time from the current time of day to the at least one predetermined time of day, and to activate the vibrating agitator following the delay time.

In some embodiments, the control unit further includes a timing device, and is adapted to provide to the capsule, as the activation input, an input signal indicating a current time of day being the at least one predetermined time of day, and the controller is adapted, upon receipt of the input signal, to activate the vibrating agitator to operate in the first vibrating mode of operation.

In some embodiments, the timing mechanism associated with the controller is a timer, and the control unit is adapted to provide to the controller, as the activation input, a delay time from the current time of day to the at least one predetermined time of day, and the controller is adapted to activate the vibrating agitator following the delay time.

In some embodiments, the current time of day is a time of day at an origin location of the subject. In other embodiments, the current time of day is a time of day at a destination location of the subject.

In some embodiments, the activation input includes the at least one predetermined time of day. In some embodiments, the activation input includes a delay time from a current time to the at least one predetermined time of day.

In some embodiments, the at least one predetermined time of day includes at least one default predetermined time of day. In some embodiments, the at least one predetermined time of day includes at least one time of day coinciding with at least one predetermined mealtime.

In some embodiments, the at least one predetermined time of day includes at least one time of day coinciding with at least one predetermined mealtime in a time zone of the origin location of the subject.

In some embodiments, the at least one predetermined time of day includes at least one time of day coinciding with at least one predetermined mealtime in a time zone of the destination location of the subject.

In some embodiments, the at least one predetermined mealtime includes at least one default mealtime.

In some embodiments, the at least one default mealtime includes a default breakfast time. In some embodiments, the default breakfast time is between 5 am and am, between 6 am and 10 am, between 6 am and 9 am, between 6 am and 8 am, between 7 am and 10 am, between 7 am and 9 am, and between 7 am and 8 am.

In some embodiments, the at least one default mealtime includes a default lunchtime. In some embodiments, the default lunchtime is between 12 pm and 3 pm, between 12 pm and 2 pm, or between 1 μm and 3 pm.

In some embodiments, the at least one default mealtime includes a default suppertime. In some embodiments, the default suppertime is between 6 pm and 10 pm, between 7 pm and 10 pm, between Bpm and 10 pm, between 6 pm and 9 pm, between 7 pm and 9 pm, or between 6 pm and 8 pm.

In some embodiments, the at least one predetermined time of day includes at least two predetermined times of day. In some embodiments, the at least two predetermined times of day include lunchtime. In some embodiments, the at least two predetermined times of day include suppertime. In some embodiments, the at least two predetermined times of day include at least two mealtimes. In some embodiments, the at least two mealtimes include lunchtime and suppertime.

In some embodiments, activation of the vibrating agitator to operate in the first vibrating mode of operation at the at least one predetermined time of day triggers a spontaneous bowel movement (SBM) in the user, which SBM occurs at a later time of day than the at least one predetermined time of day.

In some embodiments, activation of the vibrating agitator to operate in the first vibrating mode of operation at the at least one predetermined time of day triggers a complete spontaneous bowel movement (CSBM) in the user, which CSBM occurs at a later time of day than the at least one predetermined time of day. In some such embodiments, the at least one predetermined time of day is lunchtime, for example between 12 pm to 3 pm or between 12 pm to 2 pm, and the later time of day is dinnertime, for example between 6 pm and 9 pm.

In some embodiments, at least one of the capsule and the control unit includes an input mechanism for receiving subject-specific input from the subject, and wherein the at least one predetermined mealtime includes at least one subject-specific mealtime of the subject.

In some such embodiments, the subject-specific mealtime of the subject is a subject-specific mealtime in the time zone of the origin location of the subject. In other such embodiments, the subject-specific mealtime of the subject is a subject-specific mealtime in the time zone of the destination location of the subject.

In some embodiments, at least one of the capsule and the control unit includes a location sensor adapted to identify a geographical region in which the capsule is located, and wherein the at least one predetermined time of day includes at least one region-specific time of day of the geographical region in which the capsule is located. In some such embodiments, the at least one region-specific time of day includes at least one region-specific mealtime of the geographical region in which the capsule is located.

In some such embodiments, the geographical region is a geographical region of the origin location of the subject. In other such embodiments, the geographical region is a geographical region of the destination location of the subject.

In some embodiments, the activation input additionally includes a vibration protocol to be used by the vibrating agitator during the first vibrating mode of operation.

In some embodiments, the controller is adapted to activate the agitation vibration mechanism to operate in the first vibrating mode of operation at the at least one predetermined time of day only if a minimum delay duration has passed between receipt of the activation input and the at least one predetermined time of day.

In some embodiments, the vibrating agitator includes at least a radial agitator adapted, in the first vibrating mode of operation, to exert radial forces on the housing, in a radial direction with respect to the longitudinal axis of the housing, thereby to cause the vibrations exerted by the housing. In some embodiments, the radial agitator includes an unbalanced weight attached to a shaft of an electric motor powered by the power supply.

In some embodiments, the vibrating agitator includes at least an axial agitator adapted, in the first vibrating mode of operation, to exert axial forces on the housing, in an axial direction with respect to the longitudinal axis of the housing, thereby to cause the vibrations exerted by the housing. In some embodiments, the axial agitator includes an electric motor powered by the power supply and an urging mechanism, associated with, and driven by, the electric motor, the urging mechanism adapted to exert the axial forces. In some embodiments, the urging mechanism is adapted to exert the axial forces in opposite directions. In some embodiments, the urging mechanism is adapted to deliver at least a portion of the axial forces in a knocking mode.

In some embodiments, the vibrating agitator is adapted in the first vibrating mode of operation, to exert radial forces on the housing in a radial direction with respect to the longitudinal axis of the housing and to exert axial forces on the housing in an axial direction with respect to the longitudinal axis of the housing, thereby to cause the vibrations exerted by the housing. In some embodiments, the vibrating agitator includes a radial agitator adapted to exert the radial forces and a separate axial agitator adapted to exert the axial forces. In other embodiments, the vibrating agitator includes a single agitator adapted to exert the radial forces and the axial forces.

In some embodiments, the housing includes first and second members, and the vibrating agitator includes a mechanism adapted to effect vibrations by moving the first member of the housing in the opposite direction relative to the second member of the housing.

In some embodiments, the vibrating mode of operation including a plurality of cycles, each of the cycles including a vibration duration followed by a repose duration, wherein the housing exerts the vibrations during the vibration duration. In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours. In some embodiments, the cumulative duration is dependent on properties of the battery.

In some embodiments, the vibrating agitator is configured such that a net force exerted by the housing on the environment is in the range of 50 grams-force to 600 grams-force.

In some embodiments, the vibrating agitator is configured to exert the forces on the housing to attain a vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, the controlling of the vibrating agitator is effected so as to effect a mechanical stimulation of the wall of the gastrointestinal tract during the at least one predetermined time of day.

In accordance with another embodiment of the present invention there is provided a gastrointestinal capsule adapted to treat a gastrointestinal tract of a subject following ingestion of the gastrointestinal capsule, the gastrointestinal capsule including:
(a) a housing;
(b) a vibrating agitator adapted such that, in a first vibrating mode of operation, the housing exerts vibrations on an environment surrounding the capsule;
(c) a power supply disposed within the housing and adapted to power the vibrating agitator;
(d) at least one sensor adapted to identify ingestion of the gastrointestinal capsule;
(e) a clock; and
(f) a controller adapted preset with at least one predetermined time of day and functionally associated with the at least one sensor and with the clock, the controller adapted, in response to receipt of input from the at least one sensor indication ingestion of the gastrointestinal capsule, using the clock, to track time to occurrence of the at least one predetermined time of day and to activate the vibrating agitator to operate in the first vibrating mode of operation at the at least one predetermined time of day.

In some embodiments, the at least one predetermined time of day is a default time of day. In some embodiments the at least one predetermined time of day is a default mealtime.

In some embodiments, the at least one predetermined time of day includes at least two predetermined times of day. In some embodiments, the at least two predetermined times of day include lunchtime. In some embodiments, the at least two predetermined times of day include suppertime. In some embodiments, the at least two predetermined times of day include at least two mealtimes. In some embodiments, the at least two mealtimes include lunchtime and suppertime.

In some embodiments the clock is set to a default time zone. In some embodiments, the at least one predetermined time of day is set according to a typical circadian cycle of subjects in the default time zone.

In some embodiments the gastrointestinal capsule further includes a location sensor functionally associated with the controller, and the controller is further adapted, upon receipt of an input from the location sensor indicating a change in location, to activate the vibrating agitator to operate in the first vibrating mode of operation at the at least one predetermined time of day in accordance with a destination time zone.

In accordance with yet another embodiment of the present invention there is provided a system for treating a gastrointestinal tract of a subject, the system including:
a gastrointestinal capsule including:
   (a) a housing;
   (b) a vibrating agitator adapted such that, in a first vibrating mode of operation, the housing exerts vibrations on an environment surrounding the capsule;
   (c) a power supply disposed within the housing and adapted to power the vibrating agitator;
   (d) a clock;
   (e) a first communication interface, such as a transceiver; and
   (f) a controller preset with at least one predetermined time of day and functionally associated with the clock and with the communication interface, the controller adapted, in response to receipt of an activation input, using the clock, to track time to occurrence of the at least one predetermined time of day and to activate the vibrating agitator to operate in the first vibrating mode of operation at the at least one predetermined time of day; and
a control unit including a second communication interface, said control unit adapted to provide the activation input to the capsule.

In some embodiments, the control unit is adapted to provide the activation input prior to the subject ingesting the gastrointestinal capsule.

In some embodiments, the control unit further includes a sensor adapted to sense the presence of the gastrointestinal capsule in or on the control unit, and the control unit is adapted to provide the activation input in response to receipt of an input from the sensor indicating that the gastrointestinal capsule is in or on the control unit, for at least a predetermined duration.

In some embodiments, the control unit further includes a user interface, and is adapted to provide the activation input in response to receipt of a user input, via the user interface, the user input indicating that the subject will soon ingest the capsule.

In some embodiments, the control unit is devoid of a timing mechanism.

In some embodiments, the at least one predetermined time of day is a default time of day. In some embodiments the at least one predetermined time of day is a default mealtime.

In some embodiments, the at least one predetermined time of day includes at least two predetermined times of day. In some embodiments, the at least two predetermined times of day include lunchtime. In some embodiments, the at least two predetermined times of day include suppertime. In some embodiments, the at least two predetermined times of day include at least two mealtimes. In some embodiments, the at least two mealtimes include lunchtime and suppertime.

In some embodiments the clock is set to a default time zone. In some embodiments, the at least one predetermined time of day is set according to a typical circadian cycle of subjects in the default time zone.

In some embodiments the gastrointestinal capsule further includes a location sensor functionally associated with the controller, and the controller is further adapted, upon receipt of an input from the location sensor indicating a change in location, to activate the vibrating agitator to operate in the first vibrating mode of operation at the at least one predetermined time of day in accordance with a destination time zone.

In accordance with a further embodiment of the present invention there is provided a system for treating a gastrointestinal tract of a subject, the system including:
a gastrointestinal capsule including:
   (a) a housing;
   (b) a vibrating agitator adapted such that, in a first vibrating mode of operation, the housing exerts vibrations on an environment surrounding the capsule;
   (c) a power supply disposed within the housing and adapted to power
   (d) a clock;
   (e) a first communication interface, such as a transceiver; and
   (f) a controller functionally associated with the clock and with the communication interface, the controller adapted, in response to receipt of an activation input including at least one predetermined time of day, using the clock, to track time to occurrence of the at least one predetermined time of day and to activate the vibrating agitator to operate in the first vibrating mode of operation at the at least one predetermined time of day; and
a control unit including a second communication interface, said control unit adapted to provide the activation input including the at least one predetermined time of day to the capsule.

In some embodiments, the control unit is adapted to provide the activation input prior to the subject ingesting the gastrointestinal capsule.

In some embodiments, the control unit further includes a sensor adapted to sense the presence of the gastrointestinal capsule in or on the control unit, and the control unit is adapted to provide the activation input in response to receipt of an input from the sensor indicating that the gastrointestinal capsule is in or on the control unit, for at least a predetermined duration.

In some embodiments, the control unit further includes a user interface, and is adapted to provide the activation input in response to receipt of a user input, via the user interface, the user input indicating that the subject will soon ingest the capsule.

In some embodiments, the control unit is devoid of a timing mechanism.

In some embodiments, the at least one time of day is preset in the control unit. In some embodiments, the at least one predetermined time of day is a default time of day. In some embodiments the at least one predetermined time of day is a default mealtime.

In some embodiments, the at least one predetermined time of day includes at least two predetermined times of day. In some embodiments, the at least two predetermined times of day include lunchtime. In some embodiments, the at least two predetermined times of day include suppertime. In some embodiments, the at least two predetermined times of day include at least two mealtimes. In some embodiments, the at least two mealtimes include lunchtime and suppertime.

In some embodiments, the control unit is further adapted to compute the at least one time of day.

In some such embodiments, the control unit further includes a user interface, and is adapted to compute the at least one time of day based on a user input received via the user interface. In some embodiments the user input includes at least one of a sleep schedule and a meal schedule of the subject.

In some embodiments, the control unit further includes a location sensor, and is adapted to compute the at least one time of day based on a location of the control unit.

In some embodiments the gastrointestinal capsule further includes a location sensor functionally associated with the controller, and the controller is further adapted, upon receipt of an input from the location sensor indicating a change in location, to activate the vibrating agitator to operate in the first vibrating mode of operation at the at least one predetermined time of day in accordance with a destination time zone.

In accordance with a further embodiment of the present invention there is provided a system for treating a gastrointestinal tract of a subject, the system including:
a gastrointestinal capsule including:
   (a) a housing;
   (b) a vibrating agitator adapted such that, in a first vibrating mode of operation, the housing exerts vibrations on an environment surrounding the capsule;
   (c) a power supply disposed within the housing and adapted to power the vibrating agitator;
   (d) a timer;
   (e) a first communication interface, such as a transceiver; and
   (f) a controller functionally associated with the timer and with the communication interface, the controller adapted, in response to receipt of an activation input including at least one time delay, using the timer, to track passage of time to completion of the time delay and to activate the vibrating agitator to operate in the first vibrating mode of operation; and
a control unit including a clock and a second communication interface, said control unit adapted to:
   compute a time delay from a current time to at least one predetermined time of day; and
   provide to the gastrointestinal capsule the activation input including the computed time delay,
wherein the controller is adapted to activate the vibrating agitator to operate in the first vibrating mode of operation at the at least one predetermined time of day.

In some embodiments, the control unit is adapted to provide the activation input prior to the subject ingesting the gastrointestinal capsule.

In some embodiments, the control unit further includes a sensor adapted to sense the presence of the gastrointestinal capsule in or on the control unit, and the control unit is adapted to provide the activation input in response to receipt of an input from the sensor indicating that the gastrointestinal capsule is in or on the control unit, for at least a predetermined duration.

In some embodiments, the control unit further includes a user interface, and is adapted to provide the activation input in response to receipt of a user input, via the user interface, the user input indicating that the subject will soon ingest the capsule.

In some embodiments, the at least one time of day is preset in the control unit. In some embodiments, the at least one predetermined time of day is a default time of day. In some embodiments the at least one predetermined time of day is a default mealtime.

In some embodiments, the control unit is further adapted to compute the at least one time of day.

In some such embodiments, the control unit further includes a user interface, and is adapted to compute the at least one time of day based on a user input received via the user interface. In some embodiments the user input includes at least one of a sleep schedule and a meal schedule of the subject.

In some embodiments, the control unit further includes a location sensor, and is adapted to compute the at least one time of day based on a location of the control unit.

In accordance with a further embodiment of the present invention there is provided a system for treating a gastrointestinal tract of a subject, the system including:
a gastrointestinal capsule including:
   (a) a housing;
   (b) a vibrating agitator adapted such that, in a first vibrating mode of operation, the housing exerts vibrations on an environment surrounding the capsule;
   (c) a power supply disposed within the housing and adapted to power
   (d) a first communication interface, such as a transceiver; and
   (e) a controller functionally associated with the communication interface, the controller adapted, in response to receipt of an activation input, to substantially immediately activate the vibrating agitator to operate in the first vibrating mode of operation; and
a control unit including a clock and a second communication interface, said control unit adapted to provide to the gastrointestinal capsule the activation input at at least one predetermined time of day, following ingestion of the gastrointestinal capsule.

In some embodiments, the at least one time of day is preset in the control unit. In some embodiments, the at least one predetermined time of day is a default time of day. In some embodiments the at least one predetermined time of day is a default mealtime.

In some embodiments, the at least one predetermined time of day includes at least two predetermined times of day. In some embodiments, the at least two predetermined times of day include lunchtime. In some embodiments, the at least two predetermined times of day include suppertime. In some embodiments, the at least two predetermined times of day include at least two mealtimes. In some embodiments, the at least two mealtimes include lunchtime and suppertime.

In some embodiments, the control unit is further adapted to compute the at least one time of day.

In some such embodiments, the control unit further includes a user interface, and is adapted to compute the at least one time of day based on a user input received via the user interface. In some embodiments the user input includes at least one of a sleep schedule and a meal schedule of the subject.

In some embodiments, the control unit further includes a location sensor, and is adapted to compute the at least one time of day based on a location of the control unit.

In some embodiments, the gastrointestinal capsule is devoid of a timing mechanism.

In accordance with a further embodiment of the present invention there is provided a method of treating the gastrointestinal tract of a subject, the method including:
(a) providing the gastrointestinal capsule as described herein;
(b) ingesting the gastrointestinal capsule; and
(c) controlling the vibrating agitator such that the first vibrating mode of operation occurs at the at least one predetermined time of day.

In accordance with another embodiment of the present invention there is provided a method of treating the gastrointestinal tract of a subject, the method including:
(a) providing a gastrointestinal capsule, adapted to transit a gastrointestinal tract of the subject, the capsule having:
   (1) a housing arranged along a longitudinal axis;
   (2) a vibrating agitator, adapted such that, in a first vibrating mode of operation, the housing exerts vibrations on an environment surrounding the capsule;
   (3) a power supply disposed within the housing and adapted to power the vibrating agitator; and
   (4) a controller adapted, to activate the vibrating agitator to operate in the first vibrating mode of operation;
(b) receiving at least one capsule activation input;
(c) ingesting the gastrointestinal capsule; and
(d) responsive to the capsule activation input, controlling the vibrating agitator such that the first vibrating mode of operation occurs at the at least one predetermined time of day.

In accordance with yet another embodiment of the present invention there is provided a method of treating the gastrointestinal tract of a subject, the method including:
(a) providing a gastrointestinal capsule, adapted to transit a gastrointestinal tract of the subject, the capsule having:
   (1) a housing arranged along a longitudinal axis;
   (2) a vibrating agitator, adapted such that, in a first vibrating mode of operation, the housing exerts vibrations on an environment surrounding the capsule;
   (3) a power supply disposed within the housing and adapted to power the vibrating agitator; and
   (4) a controller adapted, to activate the vibrating agitator to operate in the first vibrating mode of operation;
(b) ingesting the gastrointestinal capsule; and
(c) when the gastrointestinal capsule is in an operative state, controlling the vibrating agitator such that the first vibrating mode of operation occurs at the at least one predetermined time of day.

In some embodiments, the method is adapted for treating an ailment of the gastrointestinal tract of the subject.

In some embodiments, the subject is travelling from an origin location to a destination location, and the method is adapted for mitigating jetlag of the subject.

In some such embodiments, the first vibrating mode of operation occurs at the at least one predetermined time of day according to the time zone of the origin location.

In other such embodiments, the first vibrating mode of operation occurs at the at least one predetermined time of day according to the time zone of the destination location.

In yet other such embodiments, the first vibrating mode of operation occurs, at a first time of day of the at least one predetermined time of day according to the time zone of the origin location, and at a second time of day of the at least one predetermined time of day according to the time zone of the destination location.

In some embodiments, the predetermined time of day is selected according to a circadian cycle of the subject.

In some embodiments, the predetermined time of day is selected according to a gastric pH profile of the subject.

In some embodiments, providing a gastrointestinal capsule includes providing the gastrointestinal capsule in an inoperative state, the method further including, at the capsule, receiving an activation input transitioning the capsule from the inoperative state to the operative state.

In some embodiments, providing the gastrointestinal capsule includes providing the gastrointestinal capsule including at least one timing mechanism, and devoid of sensors for sensing an environment thereof. In some such embodiments the at least one timing mechanism comprises a clock. In other embodiments the at least one timing mechanism comprises a timer.

In some embodiments, the controlling includes, responsive to the activation input, waiting a predetermined delay time, and following the predetermined delay time, at a time coinciding with the at least one predetermined time of day, activating the vibrating agitator to operate in the first vibration mode of operation.

In some embodiments, receiving the at least one activation input includes receiving the at least one activation input from at least one sensor forming part of the gastrointestinal capsule.

In some embodiments, the at least one sensor includes an illumination sensor, and the receiving the at least one activation input includes receiving input indicating transition of the capsule from an illuminated environment to a dark environment.

In some embodiments, the at least one sensor includes a pressure sensor, and the receiving the at least one activation input includes receiving input indicating pressure applied to the capsule, which pressure is indicative of the capsule moving through a pharynx of the subject.

In some embodiments, the at least one sensor includes a temperature sensor, and the receiving the at least one activation input includes receiving input indicating transition of the capsule from an area with ambient temperature to an area with a human body temperature.

In some embodiments, the at least one sensor includes an accelerometer, and the receiving the at least one activation input includes receiving the activation input in response to a detected activation motion carried out with the gastrointestinal capsule.

In some embodiments, the at least one sensor includes a moisture sensor, and the receiving the at least one activation input includes receiving input indicating transition of the capsule from a dry environment to a humid environment.

In some embodiments, the capsule further including a timing mechanism, the method further including, in response to the receiving the activation input, activating operation of the timing mechanism to track a time of day so as to identify the at least one predetermined time of day for activation of the vibration agitator.

In some embodiments, receiving the activation input includes receiving the activation input from a control unit remote from the gastrointestinal capsule.

In some embodiments, receiving the activation input includes receiving the activation input following the ingesting. In other embodiments, receiving the activation input includes receiving the activation input prior to the ingesting.

In some embodiments, receiving the activation input includes receiving a current time of day, and the controlling the vibration agitator includes computing a delay time from the current time of day to the at least one predetermined time of day and activating the vibrating agitator to operate in the first vibration mode of operation following the delay time.

In some embodiments, the activation input indicates that a current time of day is the at least one predetermined time of day, and controlling the vibration agitator to operate in the first vibration mode of operation occurs immediately following the receiving of the activation input.

In some embodiments, receiving the activation input includes receiving an indication of a delay time from the current time to the at least one predetermined time of day and activating the vibrating agitator to operate in the first vibration mode of operation following the delay time. In some such embodiments the timing mechanism of the capsule comprises a timer.

In some embodiments, the current time of day is the current time of day in the origin location of the subject. In other embodiments, the current time of day is the current time of day in the destination location of the subject.

In some embodiments, the activation input includes the at least one predetermined time of day.

In some embodiments, the at least one predetermined time of day includes at least one default predetermined time of day. In some embodiments, the at least one predetermined time of day includes at least one time of day coinciding with at least one predetermined mealtime.

In some embodiments, the at least one predetermined mealtime is a predetermined mealtime in the time zone of the origin location of the subject. In other embodiments, the at least one predetermined mealtime is a predetermined mealtime in the time zone of the destination location of the subject.

In some embodiments, the at least one predetermined mealtime includes at least one default mealtime.

In some embodiments, the at least one default mealtime includes a default breakfast time. In some embodiments, the default breakfast time is between 5 am and am, between 6 am and 10 am, between 6 am and 9 am, between 6 am and 8 am, between 7 am and 10 am, between 7 am and 9 am, and between 7 am and 8 am.

In some embodiments, the at least one default mealtime includes a default lunchtime. In some embodiments, the default lunchtime is between 12 pm and 3 pm, between 12 pm and 2 pm, or between 1 pm and 3 pm.

In some embodiments, the at least one default mealtime includes a default suppertime. In some embodiments, the default suppertime is between 6 pm and 10 pm, between 7 pm and 10 pm, between Bpm and 10 pm, between 6 pm and 9 pm, between 7 pm and 9 pm, or between 6 pm and 8 pm.

In some embodiments, the at least one predetermined time of day includes at least two predetermined times of day. In some embodiments, the at least two predetermined times of day include lunchtime. In some embodiments, the at least two predetermined times of day include suppertime. In some embodiments, the at least two predetermined times of day include at least two mealtimes. In some embodiments, the at least two mealtimes include lunchtime and suppertime.

In some embodiments, activation of the vibrating agitator to operate in the first vibrating mode of operation at the at least one predetermined time of day triggers a spontaneous bowel movement (SBM) in the user, which SBM occurs at a later time of day than the at least one predetermined time of day.

In some embodiments, activation of the vibrating agitator to operate in the first vibrating mode of operation at the at least one predetermined time of day triggers a complete spontaneous bowel movement (CSBM) in the user, which CSBM occurs at a later time of day than the at least one predetermined time of day.

In some such embodiments, the at least one predetermined time of day is lunchtime, for example between 12 pm to 3 pm or between 12 pm to 2 pm, and the later time of day is dinnertime, for example between 6 pm and 9 pm.

In some embodiments, the method further includes, prior to the controlling, receiving subject-specific input relating to at least one subject-specific mealtime of the subject, and wherein the at least one predetermined mealtime includes the at least one subject-specific mealtime.

In some embodiments, the at least one subject-specific mealtime is a subject-specific mealtime in the time zone of the origin location of the subject. In other embodiments, the at least subject-specific mealtime is a subject-specific mealtime in the time zone of the destination location of the subject.

In some embodiments, the method further includes, prior to the controlling, receiving regional information relating to a geographical region in which the gastrointestinal capsule is located, and wherein the at least one predetermined time of day includes at least one region-specific time of day of the geographical region. In some embodiments, the at least one region-specific time of day comprises a region-specific mealtime of the geographical region.

In some embodiments, the geographical region is a geographical region of the origin location of the subject. In other embodiments, the geographical region is a geographical region of the destination location of the subject.

In some embodiments, receiving the regional information includes receiving an identification of the geographical region. In some embodiments, the identification of the geographical region is received from a location sensor. In some embodiments, receiving the regional information includes receiving the at least one region-specific time of day of the geographical region. In some embodiments, receiving the regional information includes receiving the at least one region-specific mealtime of the geographical region.

In some embodiments, receiving regional information occurs in a control unit remote from the gastrointestinal capsule prior to the gastrointestinal capsule receiving the activation input, and wherein the receiving the activation input includes receiving activation input being on the received regional information.

In some embodiments, receiving regional information is carried out by the controller of the gastrointestinal capsule.

In some embodiments, receiving the activation input additionally includes receiving a vibration protocol to be used by the controller to control operation of the vibrating agitator.

In some embodiments, the vibrating agitator includes at least a radial agitator, and the controlling includes controlling the radial agitator, in the first vibrating mode of operation, to exert radial forces on the housing, in a radial direction with respect to the longitudinal axis of the housing, thereby to cause the vibrations exerted by the housing.

In some embodiments, the vibrating agitator includes at least an axial agitator, and the controlling includes controlling the axial agitator, in the first vibrating mode of operation, to exert axial forces on the housing, in an axial direction with respect to the longitudinal axis of the housing, thereby to cause the vibrations exerted by the housing.

In some embodiments, the controlling includes controlling the vibrating agitator, in the first vibrating mode of operation, to exert radial forces on the housing in a radial direction with respect to the longitudinal axis of the housing and to exert axial forces on the housing in an axial direction with respect to the longitudinal axis of the housing, thereby to cause the vibrations exerted by the housing.

In some embodiments, the vibrating agitator includes a radial agitator adapted to exert the radial forces and a separate axial agitator adapted to exert the axial forces.

In some embodiments, the vibrating agitator includes a single agitator adapted to exert the radial forces and the axial forces.

In some embodiments, the housing includes first and second members, and controlling the vibrating agitator includes effecting a vibration by moving the first member of the housing in the opposite direction relative to the second member of the housing.

In some embodiments, controlling the vibrating agitator includes controlling the vibrating mode of operation to include a plurality of cycles, each of the cycles including a vibration duration followed by a repose duration, wherein the housing exerts the vibrations during the vibration duration.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, controlling the vibrating agitator includes controlling the vibrating agitator such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours. In some embodiments, the cumulative duration is dependent on properties of the battery.

In some embodiments, in the first vibration mode of operation, the vibrating agitator is configured such that a net force exerted by the housing on the environment is in the range of 50 grams-force to 600 grams-force.

In some embodiments, in the first vibration mode of operation the vibrating agitator is configured to exert the forces on the housing to attain a vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, controlling of the vibrating agitator includes controlling the vibrating agitator so as to effect a mechanical stimulation of the wall of the gastrointestinal tract during the at least one predetermined time of day.

In accordance with another embodiment of the present invention, there is provided a method of treating the gastrointestinal tract of a subject, the method including ingesting, by the subject, a gastrointestinal capsule including:
a housing;
a vibrating agitator adapted such that, in a first vibrating mode of operation, the housing exerts vibrations on an environment surrounding the gastrointestinal capsule;
a power supply disposed within the housing, and adapted to power the vibrating agitator;
at least one sensor adapted to identify ingestion of the gastrointestinal capsule;
a clock; and
a controller preset with at least one predetermined time of day and functionally associated with the at least one sensor and with the clock, the controller adapted, in response to receipt of input from the at least one sensor indicating ingestion of the gastrointestinal capsule, to activate the vibrating agitator to operate in the first vibrating mode of operation at the at least one predetermined time of day.

In accordance with yet another embodiment of the present invention, there is provided a method of treating the gastrointestinal tract of a subject, the method including:
providing to the subject, or the subject obtaining, a gastrointestinal capsule including:
   (a) a housing;
   (b) a vibrating agitator adapted such that, in a first vibrating mode of operation, the housing exerts vibrations on an environment surrounding the capsule;
   (c) a power supply disposed within the housing and adapted to power the vibrating agitator;
   (d) a clock;
   (e) a first communication interface, such as a transceiver; and
   (f) a controller preset with at least one predetermined time of day and functionally associated with the clock and with the communication interface, the controller adapted, in response to receipt of an activation input, using the clock, to track time to occurrence of the at least one predetermined time of day and to activate the vibrating agitator to operate in the first vibrating mode of operation at the at least one predetermined time of day;
triggering, by the subject, a control unit to provide the activation input to the controller; and
ingesting, by the subject, of the gastrointestinal capsule,
such that the controller activates the vibrating agitator to operate in the first vibrating mode of operation at the at least one predetermined time of day following receipt of the activation input.

In some embodiments, the triggering comprises placing the gastrointestinal capsule in or on the control unit for at least a predetermined duration.

In some embodiments, the triggering comprises providing a user input, to the control unit, via a user interface. In some such embodiments the user input is indicative of the subject being ready to ingest the capsule.

In accordance with a further embodiment of the present invention, there is provided a method of treating the gastrointestinal tract of a subject, the method including:
providing to the subject, or the subject obtaining, a gastrointestinal capsule including:
   (a) a housing;
   (b) a vibrating agitator adapted such that, in a first vibrating mode of operation, the housing exerts vibrations on an environment surrounding the capsule;
   (c) a power supply disposed within the housing and adapted to power
   (d) a clock;
   (e) a first communication interface, such as a transceiver; and
   (f) a controller functionally associated with the clock and with the communication interface, the controller adapted, in response to receipt of an activation input including at least one predetermined time of day, using the clock, to track time to occurrence of the at least one predetermined time of day and to activate the vibrating agitator to operate in the first vibrating mode of operation at the at least one predetermined time of day;
triggering, by the subject, a control unit to provide the activation input including the at least one predetermined time of day to the controller; and
ingesting, by the subject, of the gastrointestinal capsule,
such that the controller activates the vibrating agitator to operate in the first vibrating mode of operation at the at least one predetermined time of day following receipt of the activation input.

In some embodiments, the triggering comprises placing the gastrointestinal capsule in or on the control unit for at least a predetermined duration.

In some embodiments, the triggering comprises providing a user input, to the control unit, via a user interface. In some such embodiments the user input is indicative of the subject being ready to ingest the capsule.

In some embodiments, the activation input further includes a vibration protocol, such that the controller activates the vibrating agitator to operate in the first vibrating mode of operation in accordance with the vibrating protocol.

In some embodiments, the at least one time of day is preset in the control unit. In some such embodiments the at least one time of day is a default time of day.

In some embodiments, the at least one time of day is provided to the control unit as part of a treatment protocol.

In some embodiments, the method further includes, at the control unit, computing the at least one predetermined time of day, prior to providing the activation input.

In some embodiments, the method further includes, at the control unit, receiving user input, and computing the at least one predetermined time of day based on the received user input.

In accordance with another embodiment of the present invention, there is provided a method of treating the gastrointestinal tract of a subject, the method including:
providing to the subject, or the subject obtaining, a gastrointestinal capsule including:
   (a) a housing;
   (b) a vibrating agitator adapted such that, in a first vibrating mode of operation, the housing exerts vibrations on an environment surrounding the capsule;
   (c) a power supply disposed within the housing and adapted to power the vibrating agitator;
   (d) a timer;
   (e) a first communication interface, such as a transceiver; and
   (f) a controller functionally associated with the timer and with the communication interface, the controller adapted, in response to receipt of an activation input providing at least one time delay, using the timer, to track passage of time to completion of the at least one time delay and to activate the vibrating agitator to operate in the first vibrating mode of operation;
triggering, by the subject, a control unit to provide the activation input including the at least one time delay to the controller; and
ingesting, by the subject, of the gastrointestinal capsule,
such that the controller activates the vibrating agitator to operate in the first vibrating mode of operation following the at least one time delay, at a time coincidental with at least one predetermined time of day.

In some embodiments, the triggering comprises placing the gastrointestinal capsule in or on the control unit for at least a predetermined duration.

In some embodiments, the triggering comprises providing a user input, to the control unit, via a user interface. In some such embodiments the user input is indicative of the subject being ready to ingest the capsule.

In some embodiments, the activation input further includes a vibration protocol, such that the controller activates the vibrating agitator to operate in the first vibrating mode of operation in accordance with the vibrating protocol.

In some embodiments, the method further includes, at the control unit, computing the at least one time delay from the current time to the at least one predetermined time of day.

In some embodiments, the at least one time of day is preset in the control unit. In some such embodiments the at least one time of day is a default time of day.

In some embodiments, the at least one time of day is provided to the control unit as part of a treatment protocol.

In some embodiments, the method further includes, at the control unit, computing the at least one predetermined time of day, prior to providing the activation input.

In some embodiments, the method further includes, at the control unit, receiving user input, and computing the at least one predetermined time of day based on the received user input.

In accordance with yet another embodiment of the present invention there is provided a method of treating the gastrointestinal tract of a subject, the method including:
providing to the subject, or the subject obtaining, a gastrointestinal capsule including:
   (a) a housing;
   (b) a vibrating agitator adapted such that, in a first vibrating mode of operation, the housing exerts vibrations on an environment surrounding the capsule;
   (c) a power supply disposed within the housing and adapted to power the vibrating agitator;
   (d) a first communication interface, such as a transceiver; and
   (e) a controller functionally associated with the communication interface, the controller adapted, in response to receipt of an activation input, to substantially immediately activate the vibrating agitator to operate in the first vibrating mode of operation;
ingesting, by the subject, of the gastrointestinal capsule,
following the ingesting, at at least one predetermined time of day, automatically providing an activation input from a control unit to the gastrointestinal capsule, thereby causing the controller to control vibrating agitator to operate in the first vibrating mode of operation at the at least one predetermined time of day.

In some embodiments, the activation input further includes a vibration protocol, such that the controller activates the vibrating agitator to operate in the first vibrating mode of operation in accordance with the vibrating protocol.

In some embodiments, the at least one time of day is preset in the control unit. In some such embodiments the at least one time of day is a default time of day.

In some embodiments, the at least one time of day is provided to the control unit as part of a treatment protocol.

In some embodiments, the method further includes, at the control unit, computing the at least one predetermined time of day, prior to providing the activation input.

In some embodiments, the method further includes, at the control unit, receiving user input, and computing the at least one predetermined time of day based on the received user input.

There is further provided in accordance with an embodiment of the present invention a gastrointestinal treatment system including a gastrointestinal capsule for vibrating in a gastrointestinal tract of a subject following ingestion of the gastrointestinal capsule, the gastrointestinal capsule including:
(a) a housing having a longitudinal axis;
(b) a vibrating agitator adapted such that, in a first vibrating mode of operation, the housing exerts vibrations on an environment surrounding the capsule;
(c) a power supply disposed within the housing and adapted to power the vibrating agitator; and
(d) a controller adapted, in response to receipt of an activation input, to activate the vibrating agitator to operate in the first vibrating mode of operation at at least two predetermined times of day.

In some embodiments, the at least two predetermined times of day are selected according to a circadian cycle of the subject.

In some embodiments, one of the at least two predetermined times of day is a lunchtime. In some embodiments, the lunchtime is a default lunchtime. In some embodiments, the lunchtime is a subject-specific lunchtime. In some embodiments, the lunchtime is a region-specific lunchtime.

In some embodiments, one of the at least two predetermined times of day is a suppertime. In some embodiments, the suppertime is a default suppertime. In some embodiments, the suppertime is a subject-specific suppertime. In some embodiments, the suppertime is a region-specific suppertime.

There is additionally provided in accordance with an embodiment of the present invention a method of treating constipation of a subject, the method including:
(a) providing to the subject a gastrointestinal capsule for ingestion thereof, the gastrointestinal capsule being adapted to transit a gastrointestinal tract of the subject, the gastrointestinal capsule having:
   (1) a housing arranged along a longitudinal axis;
   (2) a vibrating agitator, the vibrating agitation mechanism adapted such that, in a first vibrating mode of operation, the housing exerts vibrations on an environment surrounding the gastrointestinal capsule;
   (3) a power supply disposed within the housing and adapted to power the vibrating agitator; and
   (4) a controller adapted to activate the vibrating agitator to operate in the first vibrating mode of operation;
(b) following ingestion of the gastrointestinal capsule by the subject, and when the gastrointestinal capsule is in an operative state, activating the vibrating agitator to operate in the first vibrating mode of operation at at least two predetermined times of day; and
(c) repeating steps (a) and (b), which together form a treatment session, one to seven times per week, thereby to treat constipation of the subject.

In some embodiments, the method further includes receiving input relating a circadian cycle of the subject, and the at least two predetermined times of day are selected according to the circadian cycle of the subject.

In some embodiments, one of the at least two predetermined times of day is a lunchtime. In some embodiments, the lunchtime is a default lunchtime. In some embodiments, the lunchtime is a subject-specific lunchtime. In some embodiments, the lunchtime is a region-specific lunchtime.

In some embodiments, one of the at least two predetermined times of day is a suppertime. In some embodiments, the suppertime is a default suppertime. In some embodiments, the suppertime is a subject-specific suppertime. In some embodiments, the suppertime is a region-specific suppertime.

There is also provided in accordance with an embodiment of the present invention a method of increasing a number of spontaneous bowel movements (SBMs) or of complete spontaneous bowel movements (CSBMs) that a subject experiences per week, the method including:
(a) obtaining a baseline number of SBMs or of CSBMs that the subject has per week;
(b) providing to the subject a gastrointestinal capsule for ingestion thereof, the gastrointestinal capsule being adapted to transit a gastrointestinal tract of the subject, the gastrointestinal capsule having:
   (1) a housing arranged along a longitudinal axis;
   (2) a vibrating agitator, the vibrating agitation mechanism adapted such that, in a first vibrating mode of operation, the housing exerts vibrations on an environment surrounding the gastrointestinal capsule;
   (3) a power supply disposed within the housing and adapted to power the vibrating agitator; and
   (4) a controller adapted to activate the vibrating agitator to operate in the first vibrating mode of operation;
(c) following ingestion of the gastrointestinal capsule by the subject, and when the gastrointestinal capsule is in an operative state, activating the vibrating agitator to operate in the first vibrating mode of operation at at least two predetermined times of day on at least two consecutive days, wherein the at least two predetermined times of day include at least two predetermined mealtimes; and
(d) repeating steps b and c, which together form a treatment session, one to seven times per week, for a period of at least two weeks.

In some embodiments, the repeating of steps b and c increases the number of SBMs or CSBMs experienced by the user, per week, by at least one additional SBM or one additional CSBM for at least two of the four weeks.

In some embodiments, the method further includes receiving input relating a circadian cycle of the subject, and the at least two predetermined times of day are selected according to the circadian cycle of the subject.

In some embodiments, one of the at least two predetermined times of day is a lunchtime. In some embodiments, the lunchtime is a default lunchtime. In some embodiments, the lunchtime is a subject-specific lunchtime. In some embodiments, the lunchtime is a region-specific lunchtime.

In some embodiments, one of the at least two predetermined times of day is a suppertime. In some embodiments, the suppertime is a default suppertime. In some embodiments, the suppertime is a subject-specific suppertime. In some embodiments, the suppertime is a region-specific suppertime.

In some embodiments, the activating of the vibrating agitator includes activating the vibrating agitator to operate in the first vibrating mode of operation for a predetermined duration at each time of activation thereof.

In some embodiments, the repeating steps b and c five times per week, for a period of at least two weeks, increases the number of SBMs or CSBMs experienced by the user, per week, by at least one additional SBM or one additional CSBM.

In some embodiments, the repeating steps b and c five times per week, for a period of at least two weeks, increases the number of SBMs or CSBMs experienced by the user, per week, by at least two additional SBMs or two additional CSBMs.

In some embodiments, the increasing of the number of SBMs or CSBMs experienced by the user per week includes improving a clinical definition criteria or clinical diagnosis of the subject.

There is further provided in accordance with an embodiment of the present invention a method of treating a gastrointestinal ailment of a subject, the method including:
(a) providing to the subject a gastrointestinal capsule for ingestion thereof, the gastrointestinal capsule being adapted to transit a gastrointestinal tract of the subject, the gastrointestinal capsule having:
   (1) a housing arranged along a longitudinal axis;
   (2) a vibrating agitator, the vibrating agitation mechanism adapted such that, in a first vibrating mode of operation, the housing exerts vibrations on an environment surrounding the gastrointestinal capsule;
   (3) a power supply disposed within the housing and adapted to power the vibrating agitator; and
   (4) a controller adapted to activate the vibrating agitator to operate in the first vibrating mode of operation;
(b) following ingestion of the gastrointestinal capsule by the subject, and when the gastrointestinal capsule is in an operative state, activating the vibrating agitator to operate in the first vibrating mode of operation at at least one predetermined time of day on at least two consecutive days; and
(c) repeating steps a and b, which together form a treatment session, one to seven times per week, thereby to treat at least one gastrointestinal ailment of the subject, wherein treating of the gastrointestinal ailment includes at least one of treating or alleviating constipation of the subject, reducing straining experienced by the subject during defecating, and increasing a Bristol stool score of stool of the subject.

In some embodiments, the method further includes receiving input relating a circadian cycle of the subject, and the at least one predetermined time of day is selected according to the circadian cycle of the subject.

In some embodiments, the at least one predetermined times of day is a lunchtime. In some embodiments, the lunchtime is a default lunchtime. In some embodiments, the lunchtime is a subject-specific lunchtime. In some embodiments, the lunchtime is a region-specific lunchtime.

In some embodiments, the at least one predetermined time of day is a suppertime. In some embodiments, the suppertime is a default suppertime. In some embodiments, the suppertime is a subject-specific suppertime. In some embodiments, the suppertime is a region-specific suppertime.

In some embodiments, the activating of the vibrating agitator includes activating the vibrating agitator to operate in the first vibrating mode of operation for a predetermined duration at each activation thereof.

In some embodiments, the repeating the steps (a) and (b) one to seven times per week includes repeating steps (a) and (b) at least twice per week on consecutive days, such that first and second the gastrointestinal capsules disposed in two different locations within the gastrointestinal tract of the subject simultaneously, and the activating the vibrating agitator to operate in the first vibrating mode of operation at the at least one predetermined time of day on two consecutive days includes activating the vibrating agitators of the first and second gastrointestinal capsules to operate in the first vibrating mode of operation, simultaneously at the two different locations, during the at least one predetermined time of day.

In some embodiments, the method further includes prior to (a), obtaining a baseline number of spontaneous bowel movements (SBM) or of complete spontaneous bowel movements (CSBM) that the subject has per week.

In some embodiments, the method further includes repeating steps a-d for at least two weeks, thereby to increase a number of SBMs or CSBMs of the subject by at least one additional SBM or at least one additional CSBM over the baseline number.

In some embodiments, the method further includes repeating steps a-d for at least two weeks, thereby to increase a number of SBMs or CSBMs of the subject by at least two additional SBMs or at least one additional CSBMs over the baseline number.

In accordance with an embodiments of the present invention, there is provided a method for treatment of a disorder in a gastrointestinal tract of a human subject, the method including:
(a) activating a first vibrating ingestible capsule to carry out a first vibration protocol defined in a first treatment protocol, the first vibration protocol including delivering vibrations to a wall of the GI tract of the subject, thereby to treat the subject;
(b) receiving feedback regarding a response of the subject to treatment in accordance with the first treatment protocol;
(c) based at least on the feedback, obtaining an updated recommendation for an updated recommended treatment protocol; and
(d) programming a second programmable vibrating ingestible capsule to implement a second vibration protocol defined in a second treatment protocol, the second treatment protocol being based on the updated recommended treatment protocol; and
(e) activating the second programmable vibrating ingestible capsule to carry out the second vibration protocol, the second vibration protocol including delivering vibrations to a wall of the GI tract of the subject, thereby to treat the subject,
wherein the updated recommendation is electronically obtained based on data included in a database, the data relating to at least one other subject.

In some embodiments, the first vibrating ingestible capsule includes a programmed capsule, programmed to implement the first vibration protocol.

In some embodiments, the first vibrating ingestible capsule includes a first programmable vibrating ingestible capsule.

In some embodiments, the method further includes, prior to (a):
(f) programming the first programmable vibrating ingestible capsule to implement the first vibration protocol.

In some embodiments, first vibration protocol includes a default vibration protocol. In some embodiments, the first treatment protocol includes a default treatment protocol.

In some embodiments, the method further includes, prior to (f):
(g) receiving from the subject initial input including subject data;
(h) obtaining an initial recommendation for an initial recommended treatment protocol based on at least one characteristic included in the subject data;
(i) generating the first treatment protocol based on the initial recommended treatment protocol,
wherein at least one of initial recommendation and the updated recommendation is electronically obtained based on data included in the database, the data relating to at least one other subject.

In some embodiments, the subject data includes at least one of demographic information, medical information, and treatment history of the subject.

In some embodiments, the data included in the database includes, for each of the at least one other subject, at least one of demographic information, medical information, and treatment history information. In some embodiments, the demographic information includes at least one of gender, age, date of birth, and geographical location. In some embodiments, the medical information includes at least one of information relating to disorders of the gastrointestinal tract, information relating to chronic diseases, and information relating to medicines taken regularly. In some embodiments, the treatment history includes at least one of information relating to treatment protocols used, and a response of the at least one other subject to treatment with the treatment protocols.

In some embodiments, obtaining the initial recommendation includes:
selecting the at least one characteristic of the subject from the received subject data;
identifying in the database a treatment protocol used for treatment of other subjects sharing the at least one characteristic with the subject; and
recommending the treatment protocol as the initial recommended treatment protocol.

In some embodiments, selecting the at least one characteristic includes selecting at least two characteristics of the subject, and wherein the identifying includes identifying in the database a treatment protocol used for treatment of other subjects sharing each of the at least two characteristics with the subject.

In some embodiments, identifying includes identifying in the database a treatment protocol successfully used for treatment of the other subjects.

In some embodiments, the method further includes, following (h) and prior to (i):
providing the initial recommended treatment protocol to a decision maker; and
obtaining from the decision maker an indication whether or not the first treatment protocol should be identical to the initial recommended treatment protocol.

In some embodiments, the method further includes, if the indication obtained from the decision maker indicates that the first treatment protocol should not be identical to the initial recommended treatment protocol:
obtaining from the decision maker at least one change to be made to the initial recommended treatment protocol; and
applying the at least one change to the initial recommended treatment protocol thereby to obtain the first treatment protocol.

In some embodiments, the decision maker is a medical practitioner.

In some embodiments, the decision maker includes an electronic decision maker. In some embodiments, the at least one change obtained from the electronic decision maker is within predetermined limits for electronically changing the initial recommended treatment protocol. In some embodiments, the predetermined limits are defined in the subject data.

In some embodiments, programming the first programmable vibrating ingestible capsule includes:
providing the first vibration protocol to a capsule control unit, functionally associated with the first programmable vibrating ingestible capsule; and
the capsule control unit programming the first programmable vibrating ingestible capsule to implement the first vibration protocol.

In some embodiments, programming includes remotely transmitting the first vibration protocol to the first programmable vibrating ingestible capsule. In some embodiments, remotely transmitting includes transmitting the first vibration protocol using a short range wireless communication method. In some embodiments, remotely transmitting the first vibration protocol includes transmitting to the first programmable vibrating ingestible capsule a list of vibration parameters for effecting the first vibration protocol. In some embodiments, remotely transmitting the first vibration protocol includes transmitting to the first programmable vibrating ingestible capsule executable code for effecting the first vibration protocol.

In some embodiments, activating the first vibrating ingestible capsule includes activating a timer of the first vibrating ingestible capsule to immediately begin effecting the first vibration protocol. In some embodiments, activating the first vibrating ingestible capsule includes activating the first vibrating ingestible capsule to identify ingestion of the first vibrating ingestible capsule by the subject, and to begin effecting the first vibration protocol immediately following identification of ingestion of the first vibrating ingestible capsule.

In some embodiments, receiving feedback includes receiving from the subject feedback indicating times at which the subject experienced bowel movements during or following the treatment in accordance with the first treatment protocol. In some embodiments, receiving feedback includes receiving from the subject feedback indicating a physical feeling experienced by the subject during or following the treatment in accordance with the first treatment protocol. In some embodiments, receiving feedback includes receiving from the subject feedback indicating at least one characteristic of fecal matter excreted by the subject during or following the treatment in accordance with the first treatment protocol.

In some embodiments, receiving feedback includes receiving, from at least one sensor, information regarding expelling of the first vibrating ingestible capsule from the body of the subject. In some embodiments, the information regarding expelling includes a time at which the first vibrating ingestible capsule was expelled from the body of the subject. In some embodiments, the information regarding expelling includes an identification of the first vibrating ingestible capsule. In some embodiments, the identification includes at least one of an identification number, an RFID, a barcode, and specific dimensions of the first vibrating ingestible capsule.

In some embodiments, receiving feedback further includes receiving from the at least one sensor information regarding at least one characteristic of fecal matter excreted from the body of the subject.

In some embodiments, the at least one sensor includes a toilet-bowl mounted sensor, and wherein the receiving the information from the sensor includes:
at the toilet-bowl mounted sensor, identifying at least one of the first vibrating ingestible capsule and fecal matter being expelled from the body of the subject;
at the toilet bowl sensor, gathering the information regarding expelling of the first vibrating ingestible capsule or of expelled fecal matter; and
transmitting the information from the toilet-bowl mounted sensor.

In some embodiments, receiving feedback includes receiving feedback from at least one of a medical practitioner or a care-giver of the subject regarding the response of the subject to treatment in accordance with the first treatment protocol.

In some embodiments, receiving feedback includes:
receiving from the subject feedback indicating at least one of times at which the subject experienced bowel movements and characteristics of fecal matter expelled during or following the treatment in accordance with the first treatment protocol; and
receiving, from at least one sensor, information indicating at least one of a time at which the first vibrating ingestible capsule was expelled from the body of the subject and characteristics of fecal matter expelled from the body of the subject; and

obtaining the updated recommendation includes:
comparing the feedback received from the subject to the information received from the at least one sensor;
assigning a reliability weight to the feedback received from the subject based on the comparison; and
taking the reliability weight of the feedback received by the user into consideration when using the feedback to obtain the updated recommendation.

In some embodiments, obtaining the updated recommendation includes:
selecting the at least one characteristic of the subject from at least one of the received subject data and the received feedback;
identifying in the database a treatment protocol used for treatment of other subjects sharing the at least one characteristic with the subject; and
recommending the treatment protocol as the updated recommended treatment protocol.

In some embodiments, selecting the at least one characteristic includes selecting from the received feedback at least one other characteristic relating to the response of the subject to the treatment with the first vibrating ingestible capsule, and wherein the identifying includes identifying in the database a treatment protocol used for treatment of other subjects who had a similar response to the treatment in accordance with the first treatment protocol.

In some embodiments, identifying includes identifying in the database a treatment protocol successfully used for treatment of the other subjects.

In some embodiments, the method further includes, following (c) and prior to (d):
providing the updated recommended treatment protocol to a decision maker; and
obtaining from the decision maker an indication whether or not the second treatment protocol should be identical to the updated recommended treatment protocol.

In some embodiments, the method further includes, if the indication obtained from the decision maker indicates that the second treatment protocol should not be identical to the updated recommended treatment protocol:
obtaining from the decision maker at least one change to be made to the updated recommended treatment protocol; and
applying the at least one change to the updated recommended treatment protocol thereby to obtain the second treatment protocol.

In some embodiments, the decision maker is a medical practitioner. In some embodiments, the decision maker includes an electronic decision maker.

In some embodiments, programming the second programmable vibrating ingestible capsule includes:
providing the second vibration protocol to a capsule control unit, functionally associated with the second programmable vibrating ingestible capsule; and
the capsule control unit programming the second programmable vibrating ingestible capsule to implement the second vibration protocol.

In some embodiments, programming the second programmable vibrating ingestible capsule includes remotely transmitting the second vibration protocol to the second programmable vibrating ingestible capsule. In some embodiments, remotely transmitting includes transmitting the second vibration protocol using a short range wireless communication method. In some embodiments, remotely transmitting the second vibration protocol includes transmitting to the second programmable vibrating ingestible capsule a list of vibration parameters for effecting the second vibration protocol. In some embodiments, remotely transmitting the second vibration protocol includes transmitting to the second programmable vibrating ingestible capsule executable code for effecting the second vibration protocol.

In some embodiments, activating the second programmable vibrating ingestible capsule includes activating a timer of the second programmable vibrating ingestible capsule to immediately begin effecting the second vibration protocol. In some embodiments, activating the second programmable vibrating ingestible capsule includes activating the second programmable vibrating ingestible capsule to identify ingestion of the second programmable vibrating ingestible capsule by the subject, and to begin effecting the second vibration protocol immediately following identification of ingestion of the second programmable vibrating ingestible capsule.

In some embodiments, the method further includes, following (c), using the received feedback together with subject data received from the subject to update the database to reflect the response of the subject to the treatment in accordance with the first treatment protocol.

In some embodiments, the method further includes, following (e), repeating steps (b)-(e).

In accordance with an embodiments of the present invention, there is provided a system for treatment of a disorder in a gastrointestinal tract of a human subject, the system including:
at least one input module adapted to receive input from a human, typically at least one of the subject, a medical practitioner treating the subject, and a care giver of the subject;
a computer readable memory adapted to store, or storing, a subject profile for the subject, the subject profile including subject data received as initial input by the at least one input module;
a database including data relating to subjects treated for disorders in the gastrointestinal tract and to treatment protocols used for the subjects;
a first vibrating ingestible capsule adapted to be activated to implement a first vibration protocol defined in a first treatment protocol, and adapted, in an operative mode, to deliver vibrations to a wall of the GI tract of the subject in accordance with the first vibration protocol, thereby to treat the subject;
a second programmable vibrating ingestible capsule adapted to be programmed and activated to implement a second vibration protocol defined in a second treatment protocol, and adapted, in an operative mode, to deliver vibrations to a wall of the GI tract of the subject in accordance with said second vibration protocol, thereby to treat the subject;
a processor, functionally associated with the at least one input module, the computer readable memory, and the database;
wherein the at least one input module is adapted to receive feedback regarding a response of the subject to treatment in accordance with the first treatment protocol,
wherein the processor is configured, based at least on the feedback, to:
   automatically obtain an updated recommendation for an updated recommended treatment protocol based on at least one of said feedback, data included in said subject profile, and data included in said database, said data included in said database relating to at least one other subject of said subjects; and
   effect programming of the second programmable vibrating ingestible capsule to implement the second vibration protocol, the second treatment protocol including the second vibration protocol being based on the updated recommended treatment protocol.

In some embodiments, the first vibrating ingestible capsule includes a programmed capsule, programmed to implement the first vibration protocol.

In some embodiments, the first vibrating ingestible capsule includes a first programmable vibrating ingestible capsule.

In some embodiments, the processor is further configured to effect programming of the first programmable vibrating ingestible capsule to implement the first vibration protocol.

In some embodiments, the first vibration protocol includes a default vibration protocol. In some embodiments, the first treatment protocol includes a default treatment protocol.

In some embodiments, the processor is further configured, prior to effecting programming of the first programmable vibrating ingestible capsule, to:
obtain an initial recommendation for an initial recommended treatment protocol based on at least one characteristic included in the subject data; and
generate the first treatment protocol based on the initial recommended treatment protocol,
wherein the processor electronically obtains at least one of initial recommendation and the updated recommendation based on data included in the database, the data relating to at least one other subject of the subjects.

In some embodiments, the subject data includes at least one of demographic information, medical information, and treatment history information of the subject. In some embodiments, the data included in the database includes, for each of the at least one other subject, at least one of demographic information, medical information, and treatment history information. In some embodiments, the demographic information includes at least one of gender, age, date of birth, and geographical location. In some embodiments, the medical information includes at least one of information relating to disorders of the gastrointestinal tract, information relating to chronic diseases, and information relating to medicines taken regularly. In some embodiments, the treatment history information includes at least one of information relating to treatment protocols used, and a response of the at least one other subject to treatment with the treatment protocols.

In some embodiments, the processor is configured to obtain the initial recommendation by:
selecting the at least one characteristic of the subject from the subject data;
identifying in the database a treatment protocol used for treatment of other subjects sharing the at least one characteristic with the subject; and
recommending the treatment protocol as the initial recommended treatment protocol.

In some embodiments, the processor is further configured to provide the initial recommended treatment protocol to a decision maker and to receive from the decision maker an indication whether or not the first treatment protocol should be identical to the initial recommended treatment protocol.

In some embodiments, the processor is further configured, if the indication received from the decision maker indicates that the first treatment protocol should not be identical to the initial recommended treatment protocol, to receive from the decision maker at least one change to be made to the initial recommended treatment protocol and to apply the at least one change to the initial recommended treatment protocol thereby to generate the first treatment protocol.

In some embodiments, the decision maker is a medical practitioner.

In some embodiments, the decision maker includes an electronic decision maker. In some embodiments, at least one change received from the electronic decision maker is within predetermined limits for electronically changing the initial recommended treatment protocol. In some embodiments, the predetermined limits are defined in the subject data.

In some embodiments, the system further includes a capsule control unit functionally associated with the processor and with the at least one second programmable vibrating ingestible capsule, the capsule control unit adapted to receive from the processor the second vibration protocol and to program the second programmable vibrating ingestible capsule to implement the second vibration protocol.

In some embodiments, the capsule control unit includes a communication module for remotely transmitting the second vibration protocol to the second programmable vibrating ingestible capsule. In some embodiments, the capsule control unit is adapted to remotely transmit the second vibration protocol using a short range wireless communication method. In some embodiments, the capsule control unit is adapted to remotely transmit to the second programmable vibrating ingestible capsule a list of vibration parameters for effecting the second vibration protocol. In some embodiments, the capsule control unit is adapted to remotely transmit to the second programmable vibrating ingestible capsule executable code for effecting the second vibration protocol.

In some embodiments, the first vibrating ingestible capsule includes a first programmable vibrating ingestible capsule, and wherein the capsule control unit is adapted to receive from the processor the first vibration protocol and to program the first programmable vibrating ingestible capsule to implement the first vibration protocol.

In some embodiments, the capsule control unit includes a communication module for remotely transmitting the first vibration protocol to the first programmable vibrating ingestible capsule. In some embodiments, the capsule control unit is adapted to remotely transmit the first vibration protocol using a short range wireless communication method. In some embodiments, the capsule control unit is adapted to remotely transmit to the first programmable vibrating ingestible capsule a list of vibration parameters for effecting the first vibration protocol. In some embodiments, the capsule control unit is adapted to remotely transmit to the first programmable vibrating ingestible capsule executable code for effecting the first vibration protocol.

In some embodiments, the feedback includes feedback received from the subject indicating times at which the subject experienced bowel movements during or following the treatment in accordance with the first treatment protocol. In some embodiments, the feedback includes feedback received from the subject indicating a physical feeling experienced by the subject during or following the treatment in accordance with the first treatment protocol. In some embodiments, the feedback includes feedback received from the subject indicating at least one characteristic of fecal matter excreted by the subject during or following the treatment in accordance with the first treatment protocol.

In some embodiments, the system further includes at least one sensor adapted to provide to the processor information regarding expelling of the first vibrating ingestible capsule from the body of the subject. In some embodiments, the information regarding expelling includes a time at which the first vibrating ingestible capsule was expelled from the body of the subject. In some embodiments, the information regarding expelling includes an identification of the first vibrating ingestible capsule. In some embodiments, the identification includes at least one of an identification number, an RFID, a barcode, and specific dimensions of the first vibrating ingestible capsule.

In some embodiments, the at least one sensor is further adapted to provide to the processor information regarding at least one characteristic of excrement excreted from the body of the subject.

In some embodiments, the sensor provides the information to the processor as the feedback.

In some embodiments, the at least one sensor includes a toilet-bowl mounted sensor. In some embodiments, the toilet-bowl mounted sensor includes:
a receptacle adapted to be mounted within a toilet bowl and to receive excrement therein, the receptacle including:
   a first side wall, a second side wall, a third side wall, and a fourth side walls, the first and fourth side walls being generally opposite one another, the second and third side walls being generally opposite one another, and the third side wall including a side wall ingress; and
   a floor surface attached to the first, second, third, and fourth side walls, the floor surface including a wire frame or a mesh including openings suitable for removal of excrement from the receptacle;
a pushing mechanism, movable relative to the receptacle and adapted to push content of the receptacle, the pushing mechanism including a movable ingress;
a capsule measuring and/or identification mechanism, adapted to at least one of measure dimensions of an ingestible capsule included in the excrement and uniquely identify the ingestible capsule;
a capsule releasing mechanism for releasing the ingestible capsule from the receptacle; and
a controller for controlling operation of the pushing mechanism, the capsule releasing mechanism, and the capsule measuring and/or identification mechanism,
wherein the capsule measuring and/or identification mechanism includes a hollow formed between the side wall ingress and the movable ingress when the pushing mechanism is adjacent the third side wall, the hollow being suitable for enclosing the capsule.

In some embodiments, motion of the pushing mechanism is adapted to apply force to the excrement in the receptacle thereby to break-down the excrement to be suitably sized for removal via the openings. In some embodiments, the pushing mechanism is adapted to sense at least one characteristic of the excrement based on an amount of the force applied to the excrement in order to break down the excrement.

In some embodiments, the hollow formed between the movable ingress and the side wall ingress has a predetermined cross section, the predetermined cross section being suitably sized to match at least one of a cross section and a diameter of a specific type of capsule expected to be expelled into the receptacle.

In some embodiments, the capsule measuring and/or identification mechanism includes at least one first sensor mounted on the third side wall and adapted to sense engagement between a surface of the pushing mechanism and the third side wall.

In some embodiments, the capsule measuring and/or identification mechanism includes at least one second sensor mounted onto the side wall ingress, and adapted to sense engagement between an exterior surface of a capsule and a surface of the side wall ingress.

In some embodiments, the capsule measuring and/or identification mechanism includes a weighing mechanism adapted to sense a weight of a capsule captured in the hollow.

In some embodiments, the capsule measuring and/or identification mechanism further includes a capsule identification system, adapted to uniquely identify a capsule captured in the hollow.

In some embodiments, the controller is adapted to receive input from the capsule measuring and/or identification mechanism relating to the ingestible capsule following capturing thereof in the hollow, and to provide information based on the input to the processor. In some embodiments, the controller is adapted to receive input from the pushing mechanism relating to at least one characteristic of the excrement, and to provide information based on the input to the processor.

In some embodiments, the feedback includes feedback received from at least one of a medical practitioner or a care-giver of the subject regarding the response of the subject to the treatment in accordance with the first treatment protocol.

In some embodiments, the processor is further configured to use the subject data and the received feedback to update the database to reflect the response of the subject to the treatment in accordance with the first treatment protocol.

In accordance with an embodiments of the present invention, there is provided a toilet-bowl mounted sensor, including:
a receptacle adapted to be mounted within a toilet bowl and to receive excrement therein, the receptacle including:
   a first side wall, a second side wall, a third side wall, and a fourth side walls, the first and fourth side walls being generally opposite one another, the second and third side walls being generally opposite one another, and the third side wall including a side wall ingress; and
   a floor surface attached to the first, second, third, and fourth side walls, the floor surface including a wire frame or a mesh including openings suitable for removal of excrement from the receptacle;
a pushing mechanism, movable relative to the receptacle and adapted to push content of the receptacle, the pushing mechanism including a movable ingress;
a capsule measuring and/or identification mechanism, adapted to at least one of measure dimensions of an ingestible capsule included in the excrement and uniquely identify the ingestible capsule;
a capsule releasing mechanism for releasing the ingestible capsule from the receptacle; and
a controller for controlling operation of the pushing mechanism, the capsule releasing mechanism, and the capsule measuring and/or identification mechanism,
wherein the capsule measuring and/or identification mechanism includes a hollow formed between the side wall ingress and the movable ingress when the pushing mechanism is adjacent the third side wall, the hollow being suitable for enclosing the ingestible capsule.

In some embodiments, the sensor further includes a seal circumferentially arranged about the receptacle, wherein the receptacle and the seal are sized to circumferentially engage and seal against the toilet bowl along an entire cross section thereof, such that all matter expelled into the toilet bowl is received in the receptacle.

In some embodiments, at least one of the first, second, third, and fourth side walls includes a plurality of water jets, functionally associated with a conduit, the water jets adapted to receive water from the conduit and to spray the water into the receptacle for washing out excrement therefrom.

In some embodiments, the openings in the floor surface are suitably sized so that the ingestible capsule cannot pass through the openings.

In some embodiments, the floor surface is angled from the second side wall toward the third side wall, such that gravity assists in directing the ingestible capsule toward the capsule measuring and/or identification mechanism.

In some embodiments, the pushing mechanism extends generally parallel to the second side wall, and is movable between the second and third side walls. In some embodiments, motion of the pushing mechanism is adapted to apply force to the excrement in the receptacle thereby to break-down the excrement to be suitably sized for removal via the openings.

In some embodiments, during the motion, the pushing mechanism is adapted to sense at least one characteristic of the excrement based on and amount of the force applied to the excrement in order to break down the excrement.

In some embodiments, the pushing mechanism includes a plurality of wire frame elements connected to one another by a connector surface.

In some embodiments, the hollow formed between the movable ingress and the side wall ingress has a hollow cross section, the cross section being suitably sized to match at least one of a cross section and a diameter of a specific type of ingestible capsule expected to be expelled into the receptacle.

In some embodiments, the capsule measuring and/or identification mechanism includes at least one first sensor mounted on the third side wall and adapted to sense engagement between a surface of the pushing mechanism and the third side wall. In some embodiments, the capsule measuring and/or identification mechanism further includes at least one second sensor mounted onto the side wall ingress, and adapted to sense engagement between an exterior surface of an ingestible capsule captured in the hollow and a surface of the side wall ingress.

In some embodiments, the capsule measuring and/or identification mechanism including a weighing mechanism adapted to sense a weight of an ingestible capsule captured in the hollow. In some embodiments, the weighing mechanism forms part of, or is mounted on, the capsule releasing mechanism.

In some embodiments, the capsule measuring and/or identification mechanism includes a capsule identification system, adapted to uniquely identify an ingestible capsule captured in the hollow. In some embodiments, the capsule identification system includes a barcode reader adapted to read a barcode printed on an exterior surface of the ingestible capsule captured in the hollow, thereby to uniquely identify the ingestible capsule. In some embodiments, the capsule identification system includes a QR-code reader adapted to read a QR-code printed on an exterior surface of the ingestible capsule captured in the hollow, thereby to uniquely identify the ingestible capsule. In some embodiments, the capsule identification system includes a text interpretation mechanism adapted to capture and interpret an identification number or text printed on an exterior surface of the ingestible capsule captured in the hollow, thereby to uniquely identify the ingestible capsule. In some embodiments, the capsule identification system includes an image processing mechanism adapted to capture and interpret an image printed on an exterior surface of the ingestible capsule captured in the hollow, thereby to uniquely identify the ingestible capsule. In some embodiments, the capsule identification system includes an RFID tag reader adapted to read an RFID tag mounted onto the ingestible capsule captured in the hollow, thereby to uniquely identify the ingestible capsule.

In some embodiments, the capsule releasing mechanism includes a hinged portal formed in the floor surface beneath the side wall ingress, such that opening of the portal releases the ingestible capsule from the hollow and from the receptacle. In some embodiments, opening and closing of the portal is controlled by the controller.

In some embodiments, the controller is adapted to receive input from the capsule measuring and/or identification mechanism relating to the ingestible capsule following capturing thereof, and to provide capsule information based on the input to a remote location. In some embodiments, the controller is adapted to receive input from the pushing mechanism relating to at least one characteristic of the excrement, and to provide excrement information based on the input to a remote location.

In some embodiments, the controller is adapted to provide at least one of the capsule information and the excrement information by wired communication with the remote location, via a communication wire extending from the controller along the toilet bowl. In some embodiments, the controller is adapted to provide at least one of the capsule information and the excrement information by wireless communication with the remote location.

In some embodiments, the capsule information provided to the remote location includes at least one of a time at which the ingestible capsule was expelled into the receptacle or captured in the hollow, dimensions of the ingestible capsule, a weight of the ingestible capsule, and an identification of the ingestible capsule.

In some embodiments, the controller is adapted to provide power to the pushing mechanism, the capsule measuring and/or identification mechanism, and the capsule releasing mechanism. In some embodiments, the controller is adapted to receive power from a power supply wire extending along the toilet bowl.

In accordance with an embodiments of the present invention, there is provided a method for measuring and identifying an ingestible capsule expelled into a toilet bowl, the method including:
mounting a sensor as described herein in the toilet bowl, such that matter expelled into the toilet bowl, including the ingestible capsule, is received in the receptacle;
breaking down excrement included in the matter so that excrement is sized to be removed from the receptacle via the openings in the floor surface;
pushing the ingestible capsule toward the third side wall thereby capturing the ingestible capsule in the hollow; and
measuring whether at least one of a cross section of the ingestible capsule matches an expected cross section and a diameter of the ingestible capsule matches an expected diameter.

In some embodiments, breaking down includes moving the pushing mechanism back and forth between the second and third side walls, thereby to break down the excrement.

In some embodiments, measuring includes weighing the ingestible capsule. In some embodiments, the method further includes, following the capturing, uniquely identifying the capsule.

In some embodiments, the method further includes reporting at least one of measurements of the ingestible capsule, a weight of the ingestible capsule, and an identity of the ingestible capsule to a remote location.

In some embodiments, reporting is only carried out if the cross section of the ingestible capsule matches the expected cross section or if the diameter of the ingestible capsule matches the expected diameter. In some embodiments, reporting is only carried out if a weight of the ingestible capsule matches an expected weight.

In some embodiments, the method further includes, following the measuring, releasing the ingestible capsule from the receptacle via the capsule releasing mechanism.

### BRIEF DESCRIPTION OF THE FIGURES

The foregoing discussion will be understood more readily from the following detailed description of the invention, when taken in conjunction with the accompanying FIGS. 1-14D), in which:

FIG. 1 is a schematic block diagram of a system for treatment of a disorder in the gastrointestinal tract of a subject according to an embodiment of the present invention;

FIGS. 2A and 2B together are a schematic flowchart of a method for treatment of a disorder in the gastrointestinal tract of a subject according to the present invention, the method utilizing the inventive system of FIG. 1;

FIGS. 3A, 3B, and 3C are, respectively, a partially cut away side plan view, a partially cut away perspective view, and a top plan view of an inventive toilet-bowl mounted sensor for capturing and identifying an ingestible capsule according to the present invention, the toilet-bowl mounted sensor forming part of the system of FIG. 1;

FIG. 4 is a sectional view of another embodiment of a receptacle, forming part of an inventive toilet-bowl mounted sensor according to the present invention, in a stand-by mode;

FIGS. 5A and 5B are, respectively, a perspective view and a sectional view of the receptacle of FIG. 4 in an operational mode;

FIG. 6 is a partial sectional view of the receptacle of FIG. 4 in a capsule capturing mode;

FIG. 7 is a sectional view of the receptacle of FIG. 4 in a capsule releasing mode;

FIG. 8 is a schematic flowchart of a method for capturing and identifying an ingestible capsule that has been expelled into a toilet bowl according to the present invention, the method utilizing the inventive toilet-bowl mounted sensor of any one of FIGS. 3A-7;

FIGS. 9A to 9F are schematic block diagram of various embodiments of gastrointestinal treatment systems including a vibrating ingestible capsule according to the present invention;

FIGS. 10A to 10F are schematic flowcharts of embodiments of methods for treating the gastrointestinal tract according to the present invention, the treatment methods being based on use of the gastrointestinal treatment systems of FIGS. 9A to 9F, respectively;

FIG. 11 is a schematic illustration of the circadian cycle of a person, including suitable times of day for the implementation of the methods of FIGS. 10A to 10F;

FIG. 12 is a graphic illustration of the gastric pH of a person, indicating suitable times of day for the implementation of the methods of FIGS. 10A to 10F;

FIGS. 13A and 13B are graphic representation of results of clinical experiments conducted using an ingestible vibrating gastrointestinal capsule as illustrated in FIG. 9A and using a method as illustrated in FIG. 10A; and

FIGS. 14A, 14B, 14C, and 14D are graphic representation of results of additional clinical experiments conducted using an ingestible vibrating gastrointestinal capsule as illustrated in FIG. 9A and using a method as illustrated in FIG. 10A.

### DESCRIPTION OF THE PREFERRED EMBODIMENTS

The principles of the inventive gastrointestinal treatment systems and methods of treating the gastrointestinal tract using the inventive gastrointestinal treatment systems, and the inventive toilet-bowl mounted sensor for capturing and identifying an ingestible capsule that has been expelled into a toilet-bowl and method of use thereof, may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For the purposes of this application, the term “subject” relates to a human subject.

For the purposes of this application, the term “vibrating ingestible capsule” relates to an ingestible capsule adapted to at least intermittently vibrate, for a cumulative duration of at least one minute, in accordance with a vibration protocol of the capsule, such that, when the capsule is disposed in the gastrointestinal (GI) tract of a subject and is in operative mode, the vibrations are delivered to a wall of the GI tract of the subject, so as to provide mechanical stimulation to the GI tract of the subject. Typically, such mechanical stimulation is provided so as to treat GI disorders such as constipation, diarrhea, gastroparesis, obesity, and the like.

For the purposes of this application, the term “programmable vibrating ingestible capsule” relates to a vibrating ingestible capsule adapted to vibrate in accordance with vibration protocol, the vibration protocol adapted to be programmed into the capsule by a capsule control unit. Specifically, the vibration protocol of a programmable vibrating ingestible capsule may be changed by re-programming the capsule to implement a different vibration protocol.

For the purposes of this application, the term “programmed vibrating ingestible capsule” relates to a vibrating ingestible capsule adapted to vibrate in accordance with a vibration protocol, the vibration protocol being programmed into the capsule, for example by the manufacturer thereof. The programmed vibration protocol is fixed, and the capsule cannot be re-programmed.

For the purposes of this application, the terms “vibrating agitator” and “vibration engine” are used interchangeably, and refer to any type of structure or mechanism that vibrates or causes elements in its vicinity to vibrate, including a motor driven agitator such as a motor driven rotor including an eccentric weight, a motor drive pendulum, and a motor driven axial agitator.

For the purposes of this application, the terms “intermittently activated vibrating agitator” and “intermittently activated vibration engine” are used interchangeably, and refer to a vibrating agitator or vibration engine that vibrates or causes elements in its vicinity to vibrate and is operative at certain times, and does not vibrate or cause elements in its vicinity to vibrate at other times, the activation times being selected by a controller or other control unit controlling the vibrating agitator.

For the purposes of this application, the term “controller”, and the equivalent term “control element” refer to a computing circuit or element for controlling operation of mechanical and/or electrical components of the capsule, which form part of the capsule. The controller includes a processor or processing unit functionally associated with a non-tangible computer readable storage medium. The storage medium stores instructions, which, when executed by the processor, carry out actions which control the operation of the mechanical and/or electrical components of the capsule. For example, the instructions may include instructions to activate operation of a vibrating agitator at a specific time, frequency, cycle, and/or for a specific duration. The controller may be functionally associated with, or may include, a transceiver for receiving input, which input may be used to trigger execution of specific instructions stored in the storage medium.

For the purposes of this application, the terms “control unit” and “capsule control unit” refer to a computing circuit or device for controlling operation of mechanical and/or electrical components of the capsule, which is remote from the capsule. The control unit is adapted to program a vibrating ingestible capsule and/or to activate a vibrating ingestible capsule. The control unit includes a processor or processing unit functionally associated with a non-tangible computer readable storage medium. The storage medium stores instructions, which, when executed by the processor, carry out actions which control the operation of the capsule or provide instructions to the controller of the capsule. For example, the instructions may include instructions to receive a vibration protocol, and to transmit or provide to the received vibration protocol, or a specific vibration protocol, to the capsule thereby to program the capsule to implement the vibration protocol. For example, the capsule control unit may provide the vibration protocol to a processor of the programmable vibrating ingestible capsule by means of remote communication, such as by short range wireless communication, and may provide the protocol as a list of parameters, as executable code, or in any other manner suitable for the capsule to implement the vibration protocol. For activation, the capsule control unit typically is adapted to transmit an activation signal to a vibrating ingestible capsule, indicating to a processor of the capsule that the vibration protocol should be initiated. In some cases, the capsule control unit is also adapted to receive a signal from the capsule, indicating that the programmed vibration protocol and/or the activation instruction have been received. As another example, the instructions may include instructions to receive feedback from the capsule. The control unit is functionally associated with, or includes, a transceiver for communication with the capsule.

For the purposes of this application, the term “short range wireless communication method” relates to any wireless communication method or protocol in which signals are communicated up to a maximum range of at most 1 kilometer, at most 500 meters, at most 300 meters, at most 200 meters, at most 100 meters, at most 50 meters, or at most 10 meters such as, for example, Bluetooth communication, Wi-Fi communication, RFID signal communication, low frequency magnetic field, and the like.

For the purposes of this application, the term “clock” relates to a mechanism capable of tracking time, in a manner that is indicative of a time of day, for example as a specific hour, minute, and/or second combination. The time of day may be relative to a specific time zone, such as Eastern Standard Time (EST) or Greenwich Mean Time (GMT). A clock may be capable of tracking a time of day relative to a time preset, by default, by a processor or controller outside of the clock, or by a user, similar to a wrist watch or wall clock set to the time of day.

For the purposes of this application, the term “timer” relates to a mechanism capable of tracking passage of time relative to a beginning time, and which is not necessarily capable of tracking time in a manner that is indicative of a time of day. If the beginning time is indicative of a time of day, the timer is a clock. In a simple timer, the beginning time is not indicative of a time of day.

For the purposes of this application, the term “simple timer” relates to a timer, which is not a clock, and is only capable of tracking passage of time in a manner which is not indicative of the time of day. In a simple timer, passage of a specific time duration, such as an hour, will always be indicated in an identical manner, regardless of differences in the beginning times. For example, a simple timer tracking an hour from 1:14:57 am or tracking an hour from 12:00:00 pm would indicate the same passage of time—1:00:00—one hour, zero minutes, zero seconds.

Thus, every clock is a timer, but not every timer is a clock. Specifically, a simple timer is not a clock.

For the purposes of this application, the term “predetermined time of day” relates to any time of day occurring within a specific predetermined duration. For example, the predetermined time of day 12:00 pm to 2:00 pm encompasses any minute or second within the two hours between 12:00 pm and 2:00 pm. Typically, the predetermined time of day is relative to a specific time zone (e.g., the local time zone where the capsule is disposed, obtained, programmed, or ingested). As such, the predetermined time of day 12:00 pm to 2:00 pm EST is different from the predetermined time of day 12:00 pm to 2:00 pm GMT. The predetermined time of day must be predetermined prior to its occurrence, but may be determined only shortly before its occurrence. Specifically, the predetermined time of day need not necessarily be a default time of day or included in factory settings, and may be computed by a controller or control unit, prior to its arrival.

For the purposes of this application, the term “within”, with respect to a predetermined duration, refers to a period including the predetermined duration, as well a default “grace period” of up to 45 minutes before and after the predetermined duration.

In some embodiments the grace period is 10, 15, 20, or 30 before and after the predetermined duration.

For the purposes of this application, a vibrating agitator is considered “to operate” at a predetermined duration if it is operative at any point in time within that predetermined duration.

For the purposes of this application, the term “vibration protocol” relates to a protocol specifying vibration parameters of an intermittently activated vibrating agitator or an intermittently activated vibration engine of a vibrating ingestible capsule. Typically, the vibration protocol relates to at least one of an activation time of day (for a first activation of the vibrating agitator) and/or to an activation delay for initiating vibration (a duration between activation of the capsule and the first activation of the vibrating agitator), a vibration rate (number of vibration cycles per hour), a vibration duration and a repose duration for each vibration cycle, a vibration frequency, an amount of force exerted by the vibrations, and the like.

For the purposes of this application, the term “treatment procedure” relates to parameters of a treatment utilizing vibrating ingestible capsules, which are typically defined by a treating physician or medical practitioner. For example, the treatment procedure may include the number of capsules to be taken within a specific time duration (e.g. 3 capsules per week, 2 capsules per day), the frequency at which capsules should be taken, the time of day at which capsules should be taken, whether the capsule should be taken with or without food, and the like.

For the purpose of this application, the term “treatment protocol” relates to all aspects of treatment of a subject with a vibrating ingestible capsule, and includes the treatment procedure as well as the vibration protocol to be used for treating the subject.

For the purposes of this application, the term “default treatment protocol” relates to a standard treatment protocol which is typically used when initially treating a subject with a vibrating ingestible capsule, or when insufficient information is available for tailoring a specific treatment protocol to the subject.

For the purposes of this application, the terms “default vibration protocol” relates to a standard vibration protocol which is typically used when initially treating a subject with a vibrating ingestible capsule, or when insufficient information is available for tailoring a specific vibration protocol to the subject. In some cases, the default vibration protocol is the vibration protocol used by programmed vibrating ingestible capsules.

For the purposes of this application, the term “electronically obtained”, when relating to a treatment protocol, relates to a treatment protocol which was generated by or determined by a machine based on electronic information, such as information included in a computerized or electronic profile of a subject and/or data included in a database, without direct human input or intervention.

For the purposes of this application, the term “successful treatment”, and variations thereof, relates to treatment of a subject with one or more capsules implementing a specific treatment protocol such that during or following the treatment, the symptoms of the subject are improved by a significant degree, as defined by medical practices. For treatment of chronic constipation, successful treatment is treatment that results in an increase of at least one bowel movement per two-weeks, an increase of at least one bowel movement per week, an increase of three bowel movements per two-weeks, or an increase of at least two bowel movements per week, on average.

For the purposes of this application, the term “decision maker” relates to any entity capable of applying logic to a proposed treatment protocol, in order to determine whether or not the proposed treatment protocol is suitable for treatment of a subject, and/or whether the proposed treatment protocol must be modified in order to be suitable for treatment of the subject. The decision maker may be a human, or a machine including suitable artificial intelligence or logic components.

For the purposes of this application, the term “electronic decision maker” relates to a decision maker which is fully based on artificial intelligence or logic, and which makes decisions regarding the suitability of a proposed treatment protocol for treatment of a subject without direct input from a human, only based on artificial logic and/or intelligence. The electronic decision maker is typically limited to decisions within a scope determined by a human, such as a medical practitioner, prior to use of the electronic decision maker. For example, a doctor may define a range of vibration protocols which is acceptable for use for a subject, and the electronic decision maker may make decisions within that range, but would refer to a human decision maker if decisions are required that are outside of the predefined range.

For the purpose of this application, the term “activation input” relates to an input received by a controller of a vibrating ingestible capsule, which causes the controller of the capsule to activate itself, so as to be able to process inputs and/or to control additional components of the capsule. The activation input may be received from an element forming part of the capsule, such as a sensor sensing specific conditions in which the capsule should be activated, or from a remote source, such as a remote control unit, for example by way of a signal transmitted to the capsule, magnetic field applied to the capsule, specific motion applied to the capsule, or any other type of input provided to the capsule from a remote source.

For the purpose of this application, a vibrating ingestible capsule is said to be in an “inoperative state” when the capsule is in a storage condition, intended to preserve the life of a battery thereof. In the inoperative state, components of the capsule which are intended to receive or to provide an activation input, such as specific sensors, transceivers, and/or timing mechanisms may be active at least to a minimal degree. However, in the inoperative state, no vibration takes place, and a controller controlling vibration of the capsule is inactive.

For the purpose of this application, a vibrating ingestible capsule is said to be in an “operative state” when the controller of the capsule is processing inputs and data, and can cause a vibrating agitator of the capsule to vibrate.

For the purpose of this application, the term “jetlag” relates to any symptom resulting from a change in time zones, including, but not limited to, a shift in a person's sleep schedule, acute constipation resulting from travel, and the like.

For the purpose of this application, the term “mitigating jetlag” relates to appreciably decreasing any jetlag symptom, for example by at least 25%. For example, for a traveler who typically suffers from acute constipation for the first 48 hours of travel, jetlag would be mitigated if the traveler suffered from such acute constipation for, at most, the first 36 hours of travel.

For the purposes of this application, the term “chronic constipation” relates to a spontaneous bowel movement (SBM) frequency of at most 3 SBMs per week,

For the purposes of this application, the term “acute constipation” relates to a subject suffering from a specific event of constipation, without necessarily suffering from chronic constipation. For example, the subject may be constipated for several days following travel, or during travel, for example as part of the subject's jetlag symptoms.

For the purposes of this application, the term “vitality schedule” relates to a timeline defining, for each time window, a vitality level of the subject, where the vitality level may reflect the subject's wakefulness and activity level at that time. For example, for a person who wakes up early and is the most productive during the early morning, but falls asleep early, the vitality schedule may include high vitality from 5 am to 11 am, medium vitality from 11 am to 8 pm, low vitality from Bpm to 4 am, and again medium vitality from 4 am to 5 am.

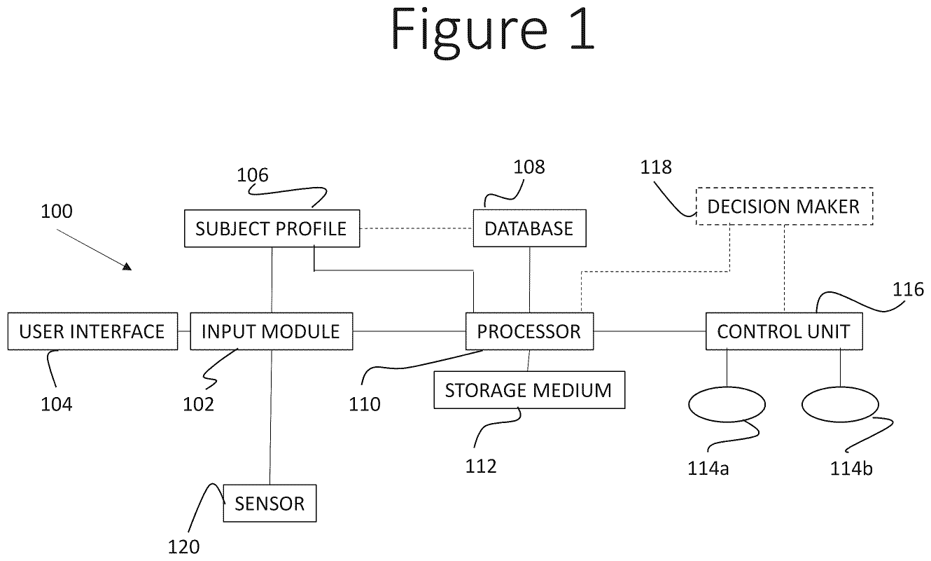

Referring now to the drawings, FIG. 1 is a schematic block diagram of a system 100 for treatment of a disorder in the gastrointestinal tract of a subject according to an embodiment of the present invention.

As seen in FIG. 1, system 100 includes at least one input module 102 operative to receive input from at least one of the subject, a medical practitioner or medical personnel treating the subject, and a care giver of the subject, such as a parent, guardian, or personal medical aide, for example via a user interface 104. The input module 102 may further be operative to receive input from one or more sensors, as described in further detail hereinbelow. In some embodiments, separate input modules 102 and/or different user interfaces 104 are used for different input providers.

The input module 102 may be any suitable input module, and may include, for example, a receiver or transceiver configured to receive the input as a communication signal from a remote location, such as from the one or more sensors, or a keyboard or touchpad configured to receive input entered directly thereinto, for example by the subject, a caretaker of the subject, medical personnel treating the subject, or any other input provider.

A computer readable memory stores a subject profile 106 for the subject, including subject data. The subject profile 106 may be functionally associated with the input module 102, and the subject data may be provided to the subject profile 106 as input, via one of more input module 102. For example, the subject, the medical practitioner or medical personnel treating the subject, or the care giver of the subject may provide the subject data, for example as an initial input.

In some embodiments, the subject data includes demographic information of the subject, such as the subject's name, identification number such as a social security number or passport number, address, date of birth, age, gender, contact information, and emergency contact information.

In some embodiments, the subject data includes medical information of the subject, such as the subject's medical history and particularly the medical history of constipation, diarrhea, gastroparesis, obesity, and/or gastrointestinal diseases, medications and/or treatments previously tried by the subject, particularly for constipation, diarrhea, gastroparesis, obesity, and/or gastrointestinal diseases, results of medical examination such as etiology of constipation, results from physical examination such as digital rectal examinations, results from gastrointestinal explorations such as transit or motility studies, anorectal manometry, balloon test expulsion, scintigraphy scan, or other gastrointestinal explorations, blood test information, stool sample information, microbiome information, information relating to allergies, chronic diseases, medications currently being used, and the like. In some embodiments, the medical information may be collected before, during, and/or after treatment in accordance with the method described herein.

In some embodiments, the subject data may also include information relating to the subject's lifestyle, such as diet, water intake, and physical activity.

In some embodiments, for female subjects, the subject data may also include obstetric medical history.

In some embodiments, the subject data may include guidelines for treatment of the subject, or a “treatment safe zone” or “self adapting zone” for the subject, such as, for example, a range of vibration protocols suitable for safe treatment of the subject, which may be used by an electronic decision maker as described in further detail hereinbelow.

In some embodiments, the system includes a database 108, which may be functionally associated with one or more input module 102 and/or with subject profile 106. Database 108 includes data relating to subjects treated for disorders in the gastrointestinal tract. In some embodiments, data is input into the database 108 directly by an input provider, such as one or more subjects, a medical practitioner, medical personnel, a researcher or administrator at a research facility, and the like, for example via input module 102 and user interface 104. In some embodiments, the data is input into the database 108 automatically, for example directly from the subject profile 106, as described in further detail hereinbelow.

In some embodiments, data included in database 108 relates to at least one other subject, and preferably to a plurality of subjects other than the subject.

In some embodiments, the data included in database 108 includes, for each other subject, demographic information such as gender, age, date of birth, and the like, as well as information relating to disorders of the gastrointestinal tract experienced by the other subject, to treatments provided for such disorders, and to the other subject's response to such treatments. In some embodiments, the data included in database 108 may be substantially parallel to the subject data. Specifically, in some embodiments, the data included in the database lists, for each of the other subjects, one or more vibration protocols, treatment procedures, and/or treatment protocols used to treat the other subject, and the other subject's response to treatment with such protocols and procedures.

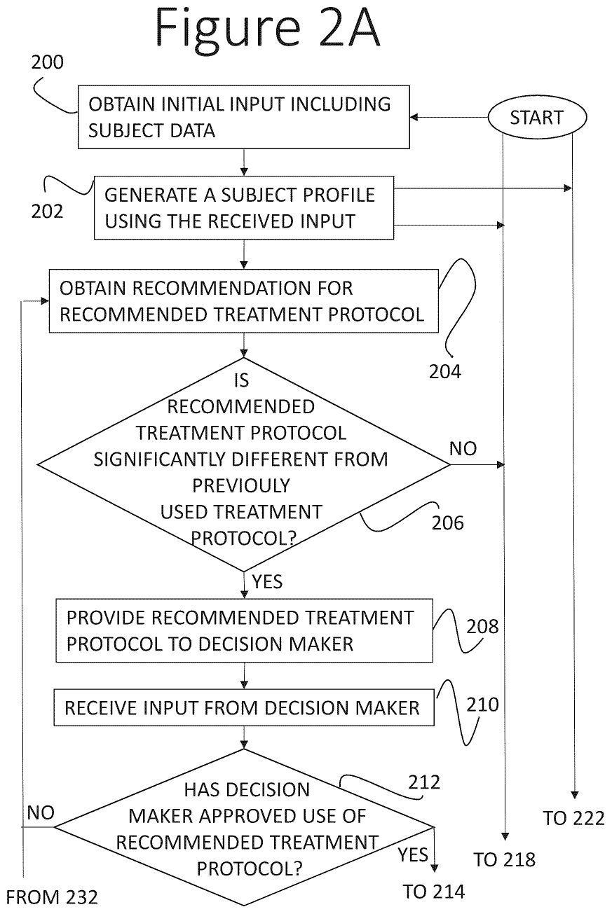

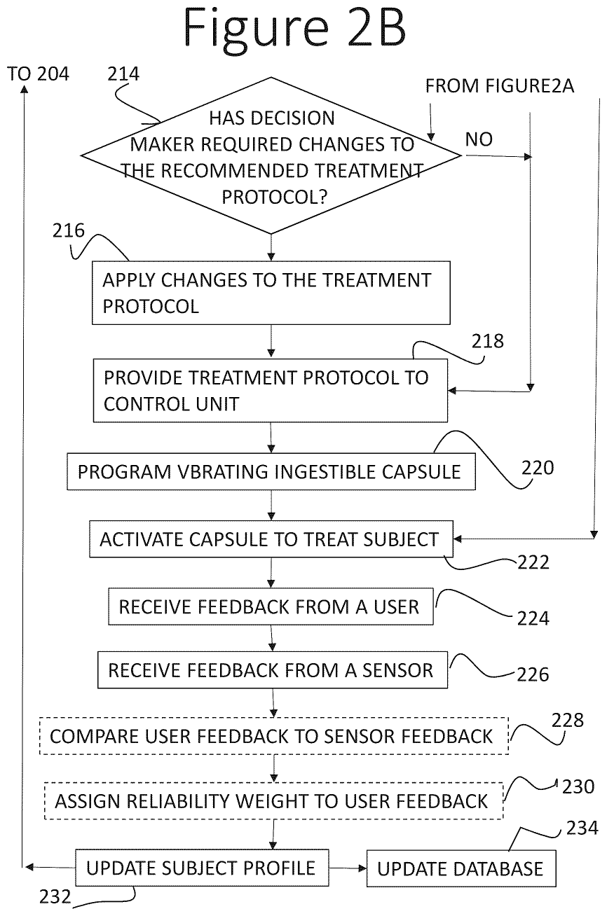

A processor 110 is functionally associated with subject profile 106 and with database 108, and may further be functionally associated with input module 102. Processor 110 is functionally associated with a computer readable storage medium 112 storing instructions, which may be carried out by the processor 110, as explained in further detail hereinbelow with respect to FIGS. 2A and 2B.

In some embodiments, processor 110 is configured to receive, for example via input module 102 or via subject profile 106, feedback relating to a response of the subject to treatment with a specific treatment protocol, a specific vibration protocol, or specific treatment procedures. For example, the feedback may indicate times at which the subject experienced bowel movements following the treatment, a physical feeling experienced by the subject during or immediately after the treatment, or one or more characteristics of fecal matter excreted by the subject during or following the treatment.

In some embodiments, the feedback is received from a human, such as from the subject, a medical practitioner or medical professional treating the subject, or a care-giver of the subject. In some embodiments, the feedback is received from a sensor, as described in further detail hereinbelow.

In some embodiments, the processor 110 is further configured to assign a weight, or a reliability score, to such feedback, as described in further detail hereinbelow.

In some embodiments, processor 110 is configured to electronically obtain an initial recommendation for an initial recommended treatment protocol for the subject. In some embodiments, the initial recommendation may be a default initial recommendation. In some embodiments, the initial recommendation may be based on at least one selected characteristic included in the subject data. In some embodiments, the initial recommendation may be based on data included in the database 108, for example data relating to other subjects sharing the selected characteristic(s) with the subject.

In some embodiments, processor 110 is configured to electronically obtain an updated recommendation for an updated recommended treatment protocol for the subject, based on received feedback relating to a response of the subject to a previously used treatment protocol, on at least one characteristic included in the subject data and/or on data included in the database 108, for example data relating to other subjects sharing the characteristic with the subject or sharing the experienced response with the subject.

The updated recommendation may change one or more parameters of the vibration protocol or of the treatment protocol, relative to a previous recommendation. For example, the updated recommendation may change the vibration frequency, the delay time until starting the vibration protocol, the duty cycle, the segment in the GI tract in which the capsule is intended to vibrate, the length of the vibration time in each vibration cycle, the rest time of each vibration cycle, and/or the vibration intensity.

The system 100 further includes a first vibrating ingestible capsule 114a, adapted to implement a first vibration protocol for delivering vibrations, or mechanical stimulation, to a wall of the GI tract of the subject thereby to treat the gastrointestinal disorder of the subject, the first vibration protocol forming part of a first treatment protocol for the subject. The system further includes at least one second programmable vibrating ingestible capsule 114b, adapted to be programmed to implement a second vibration protocol delivering vibrations, or mechanical stimulation, to a wall of the GI tract of the subject thereby to treat the gastrointestinal disorder of the subject, the second vibration protocol forming part of a second treatment protocol for the subject. As explained in further detail hereinbelow, in some embodiments, the first and/or second vibration protocols and/or treatment protocols may be identical to, or may be based on, recommended vibration protocols and/or treatment protocols electronically obtained by processor 110.

Each of capsules 114a and 114b may include a sensor module including one or more sensors; a timer; an intermittently activated vibration engine; a processor, functionally associated with the sensor(s) in the sensor module, with the timer, and with the vibration engine; a receiver or transceiver functionally associated with the processor; and at least one power source providing power to the sensor module, the timer, the vibration engine, the processor, and/or the receiver/transceiver.

In some embodiments, the sensors included in the sensor module may sense conditions in the vicinity of the capsule. As such, the sensors may include: a pH sensor adapted to sense the pH in the vicinity of the capsule; a light sensor adapted to sense a degree of illumination in the vicinity of the capsule; a pressure sensor adapted to sense pressure applied to the capsule; and/or an orientation sensor, such as an accelerometer, adapted to sense the three dimensional orientation of the capsule.

The power source of capsules 114a and 114b may be any suitable power source, such as, for example, one or more alkaline or silver oxide batteries, lithium batteries, primary batteries, rechargeable batteries, capacitors and/or super capacitors.

In some embodiments, the capsules 114a and 114b may be substantially as described in U.S. Patent Application Publication No. 2015/0073315 and in U.S. Pat. No. 9,078,799 which are incorporated by reference as if fully set forth herein.

In some embodiments, first vibrating ingestible capsule 114a may be a programmed vibrating ingestible capsule, programmed, for example at a time of manufacturing thereof, to implement a specific vibration protocol as the first vibration protocol. In other embodiments, the first vibrating ingestible capsule 114a is a first programmable vibrating ingestible capsule 114a, adapted to be programmed to implement the first vibration protocol.

In some embodiments, the first treatment protocol may be a default treatment protocol, used as the standard initial treatment protocol for subjects suffering from any disorder, or from a specific disorder, of the gastrointestinal tract, and may define a default vibration protocol as the first vibration protocol.

A capsule control unit 116, functionally associated with processor 110 and with at least one programmable vibrating ingestible capsule, is adapted to receive a vibration protocol and to program a programmable vibrating ingestible capsule to implement the received vibration protocol. In some embodiments, the capsule control unit 116 is adapted to receive a treatment protocol including treatment procedures and a vibration protocol and to program the programmable vibrating ingestible capsule to implement the received vibration protocol.

In some embodiments, capsule control unit 116 may include a dedicated processor; a programming module; a verification and activation module; and/or a transceiver or other communication module. The capsule control unit may further include at least one power source providing power to components of the capsule control unit.

In some embodiments, the capsule control unit 116 further includes a user interface, adapted to provide output to the subject or to another user associated with the subject, such as a medical practitioner or care giver. In some such embodiments, the capsule control unit 116 may prompt the subject, via the user interface, to ingest one or more capsules in accordance with the treatment protocol received by the capsule control unit.

Capsule control unit 116 may be any suitable control unit, including a suitably programmed computing device such as a smartphone, a tablet computer, laptop computer, desktop computer, and the like, or may be a dedicated control unit, e.g. a stand-alone control unit or a control unit forming part of another medical device.

In system 100, capsule control unit 116, or a dedicated processor thereof, is adapted to receive from processor 110 the second treatment protocol, for example via a transceiver of the capsule control unit 116, and to program the second programmable vibrating ingestible capsule 114b to implement the second vibration protocol defined in the second treatment protocol. In some embodiments, in which first vibrating ingestible capsule 114a is programmable, capsule control unit 116 may be further adapted to similarly receive the first treatment protocol, from processor 110 or from another source, and to program the first vibrating ingestible capsule 114a to implement the first vibration protocol defined in the first treatment protocol.

In some embodiments, the programming module of capsule control unit 116 is configured to receive the second vibration protocol from the processor of the capsule control unit, and to program a processor of second programmable vibrating ingestible capsule 114b to implement the second vibration protocol upon activation thereof or upon ingestion of the second vibrating ingestible capsule. Similarly, in embodiments in which first vibrating ingestible capsule 114a is programmable, the programming module of capsule control unit 116 is configured to receive the first vibration protocol from the processor of the capsule control unit, and to program a processor of first vibrating ingestible capsule 114a to implement the first vibration protocol upon activation thereof or upon ingestion of the first vibrating ingestible capsule.

In some embodiments, the programming module of capsule control unit 116 may include a transmitter, or may be functionally associated with a transmitter, for transmission of the first and/or second vibration protocol to a receiver/transceiver of capsules 114a and/or 114b, for example using a short range wireless communication method.

In some embodiments, the programming module of capsule control unit 116 transmits the vibration protocol to the capsule 114a or 114b as a sequence of parameter signals, such as digits, indicating time frames from activation or from ingestion and vibration parameters to be used at those time frames. In some embodiments, the programming module of capsule control unit 116 transmits the vibration protocol to the capsule 114a or 114b as executable code to be used by the processor of the capsule to automatically activate the vibration engine thereof in accordance with the vibration protocol.

In some embodiments, a memory element within the capsule 114a or 114b includes multiple vibration protocols which may be effected by the capsule, and the programming module of capsule control unit 116 transmits to the capsule an indication of a specific one of the multiple vibration protocols to be used, such as a vibration protocol identifier.

In some embodiments, the activation module of the capsule control unit 116 is configured to activate the first and/or second programmable vibrating ingestible capsule for operation.

In some cases, the activation includes activating the capsule to identify ingestion thereof by the subject, and subsequently to begin implementation of the vibration protocol programmed therein. In other embodiments, activation of the capsule includes immediate activation of the timer to start counting time till activation of the vibration engine. Such embodiments assume that the capsule is activated shortly before the subject ingests the capsule. In yet other embodiments, activation of the capsule occurs following ingestion of the capsule by the subject, by providing to the ingested capsule a remote activation signal.

In some embodiments, the activation module of capsule control module 116 includes a transmitter, or is functionally associated with a transmitter or transceiver, and activates the capsule by providing an activation signal thereto.

In some embodiments, the activation module also includes a verification module, configured to verify that the capsule received the vibration protocol transmitted thereto and is capable of effecting the vibration protocol without error. In some embodiments, the verification module includes, or is functionally associated with a receiver, receiving from the capsule a verification signal indicating that the capsule has received the vibration protocol. In some embodiments, the verification module includes a vibration sensor, sensing the capsule vibrating in accordance with a specific vibration pattern indicative of verification thereof.

In some embodiments, the processor 110 and the capsule control unit 116 are functionally associated with a decision maker 118, which is adapted to receive, for example from processor 110, a suggestion or recommendation for a treatment protocol or a vibration protocol, and to approve, reject, or modify the suggested or recommended protocol for treatment of the subject.

In some embodiments, the decision maker may be a human decision maker, such as a medical practitioner or treating gastroenterologist, who may receive the suggested or recommended treatment protocol or vibration protocol by the protocol being presented on a display visible to the human decision maker, or by receiving an electronic message, such as an email message, specifying the recommended or suggested treatment protocol or vibration protocol.

The human decision maker may approve, reject, or modify the received recommended treatment protocol or vibration protocol by interacting with a user interface 104 of one of input modules 102, or by interacting directly with capsule control unit 116, for example with an input module or user interface thereof.

In other embodiments, the decision maker 118 is an electronic decision maker, such as a suitably equipped computer or server, which is adapted to electronically receive the suggested or recommended treatment protocol or vibration protocol, and to approve, reject, or modify the suggested or recommended treatment protocol or vibration protocol. In some embodiments, the logic applied by the electronic decision maker may be learned over time, for example based on the results that similar treatment had for other subjects. In some embodiments, the electronic decision maker may include an artificial intelligence component.

In some embodiments, decisions made by an electronic decision maker are limited to be within certain limitations, or within a “self adapting zone” predefined by a human decision maker, such as safety regulations defined by a manufacturer of the capsule and/or medical limitations defined by a medical practitioner. For example, the manufacturer may indicate that the frequency of vibrations has an upper limit of 1000 Hz, in which case the decision maker will not permit, or approve, any protocol requiring vibration at a frequency greater than 1000 Hz. As another example, a medical practitioner may indicate that no changes can be made to treatment procedures, in which case the electronic decision maker would make decisions relating to the vibration protocol, but would defer any decisions relating to the treatment procedures to a human decision maker, for example via an electronic message.

In some embodiments, the limitations are based on the structure and/or functionality of the capsule, such as safety limitations defined by the manufacturer. In such embodiments, the same limitations apply to multiple subjects, and multiple capsules. In some embodiments, the limitations are defined by the medical practitioner based on the medical state of the subject, and are specific to the subject being treated. Such limitations may be provided as input by the medical practitioner, for example as part of the subject data stored in the subject profile.

In some embodiments, the electronic decision maker may be functionally associated with the capsule control unit 116, and may transmit the approved or modified treatment protocol or vibration protocol directly to the capsule control unit 116. In other embodiments, the electronic decision maker provides the approved or modified treatment protocol or vibration protocol to processor 110, which may transmit the treatment protocol to capsule control unit 116 as described hereinabove.

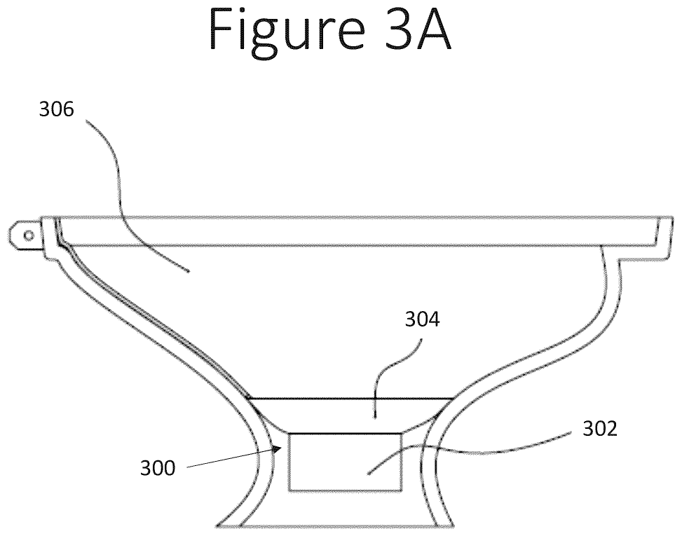

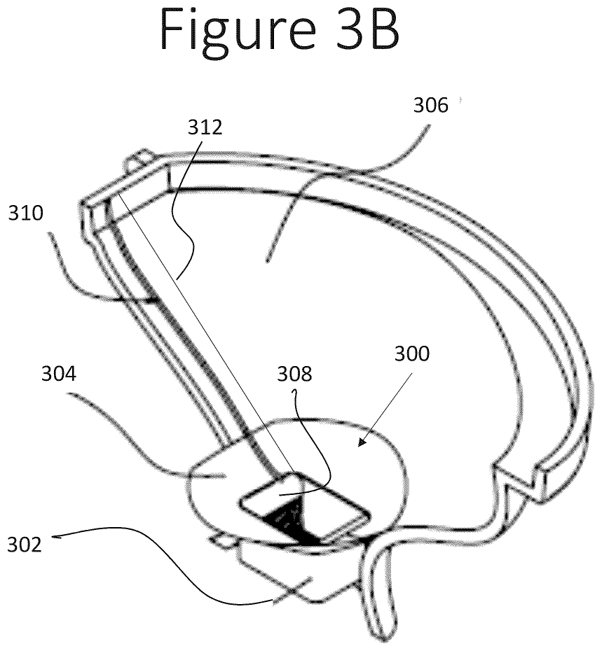

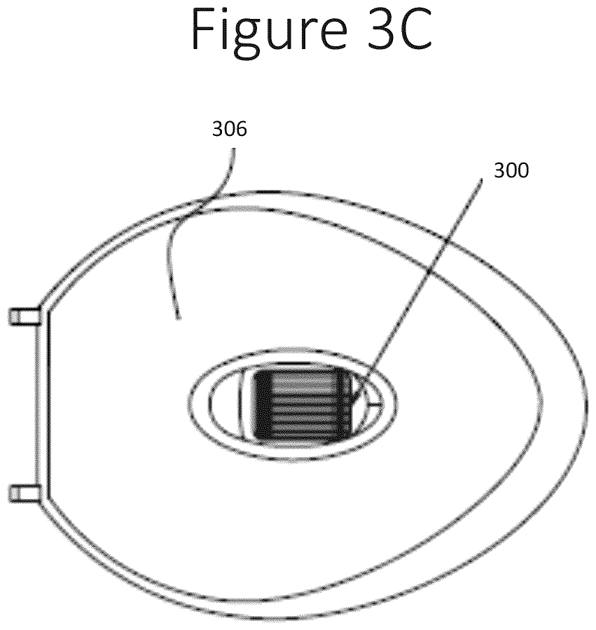

In some embodiments, processor 110 and/or subject profile 106 are further associated with at least one sensor 120, which is adapted to sense, and provide to processor 110 and/or to subject profile 106, information regarding a vibrating ingestible capsule being expelled from the body of the subject. In some embodiments, the sensor comprises a toilet-bowl mounted sensor. The toilet bowl mounted sensor may be any suitable sensor, such as any one of the inventive sensors described hereinbelow with reference to FIG. 3A to 8, or a toilet-bowl mounted sensor known in the art, for example as described in US Patent Application Publication No. 2009/0326514, which is hereby incorporated by reference.

The information provided by sensor 120 may include a time at which the vibrating ingestible capsule was expelled from the body of the subject, an identification of the expelled capsule, and/or information regarding characteristics of excrement expelled from the body of the subject.

Reference is now additionally made to FIGS. 2A and 2B, which, together, are a schematic flowchart of a method for treatment of a disorder in the gastrointestinal tract of a subject according to the present invention, the method utilizing the inventive system of FIG. 1.

As seen, and as described in further detail hereinbelow, the method may start at step 200, at step 218, or at step 222.

In some embodiments, the method begins at step 200, by obtaining initial input including subject data for a subject to be treated using the method. The subject data typically includes demographic information and/or medical history information for the subject, required for initiating subject profile 106. In some embodiments, the input may additionally include information relating to other subjects, such as information required for creating or updating database 108. In some embodiments, the input may further include limitations defining a “self adapting zone” for an electronic decision maker, as described hereinabove.

As mentioned hereinabove with reference to FIG. 1, the input may be received from the subject, from a medical facility or medical practitioner treating the subject, or from a caretaker of the subject. In some embodiments, the input is provided to input module 102 via a user interface 104 which is local to system 100, while in other embodiments the input may be provided via a remote user interface 104, and communicated to input module 102 or to processor 110 using a suitable communication method.

At step 202, a subject profile, such as subject profile 106 described hereinabove with respect to FIG. 1, is generated for the subject, for example by processor 110, based on the input relating to the subject.

Turning to step 204, it is seen that a recommendation for a recommended treatment protocol for the subject is obtained. In some embodiments, the recommendation is electronically obtained by processor 110. In some embodiments, the recommendation is based on information included in the subject profile 106.

For example, in an initial treatment round, the subject profile may indicate that the motility in the subject's digestive system is slower than the motility of the average digestive system, such that the food reaches the colon after a longer time than the average hours. In such embodiments, processor 110 may recommend a treatment protocol having a long enough activation delay time so as to ensure that the capsule vibrates when it has reached the subject's large intestine, and does not vibrate prematurely.

In some embodiments the recommendation may also be based on data obtained from database 108, the data relating to one or more other subjects. For example, in an initial treatment round, the subject profile 106 may indicate that the subject is a female aged 55. Processor 110 may access database 108 to identify treatment protocols and/or vibration protocols that were successfully used to treat other females having similar ages, for example aged 50-60. One such identified protocol may be suggested as the recommended treatment protocol for the subject, or a new treatment protocol, including characteristics of a number of such identified protocols, may be generated as the recommended treatment protocol for the subject.

As described in further detail hereinbelow, the method described herein is iterative, and subject profile 106 may be updated to reflect the response of the subject to treatment with a specific treatment protocol. As such, in subsequent treatment rounds, processor 110 may use data relating to such responses when generating the recommendation for the next treatment protocol to be used.

Returning to the example provided above, the subject profile 106 may, in a later treatment round, indicate that the subject's condition did not improve when treated with a vibration protocol providing capsule vibration at a frequency of 150 Hz. The processor may then recommend an updated vibration protocol having a different vibration frequency.

Selection of a subsequent treatment protocol may also be based on data from the database, relating to at least one other user. In the example above in which the subject's condition did not improve when treated with a vibration protocol providing capsule vibration at a frequency of 150 Hz, the processor may, when identifying in database 108 potential treatment protocols, limit the identified protocols to ones providing vibration at a higher frequency, provided that the higher frequency is within a predetermined permissible range for the capsule and/or for the specific subject, such as up to 250 Hz. As another alternative, the processor may identify treatment protocols that were successfully used to treat females aged 50-60 which did not respond well to treatment with capsule vibration at a frequency of 150 Hz.

When updating the vibration protocol or treatment protocol in a second or subsequent treatment round, the updated recommended treatment protocol may change one or more parameters of the vibration protocol or of the treatment protocol, relative to a previous recommendation. For example, the updated recommendation may change parameters of the vibration protocol, such as the vibration frequency, the delay time until starting the vibration protocol, the duty cycle, the segment in the GI tract in which the capsule is intended to vibrate, the length of the vibration time in each vibration cycle, the rest time of each vibration cycle, and/or vibration intensity, and/or parameters of the treatment procedures, such as the frequency at which a capsule should be ingested, the time of day at which the capsule should be ingested, and/or whether the capsule should ingested with or without food.

As seen at step 206, the processor 110 may then check whether or not the obtained recommended treatment protocol is significantly different from a previously used treatment protocol. Naturally, in an initial iteration of the method described herein, step 206 would be obviated. In some embodiments, a treatment protocol would be considered ‘significantly different’ from another treatment protocol if the vibration protocol and/or the treatment procedures defined by the treatment protocol, or any portion thereof, is not in the range of allowed treatment protocols and/or characteristics defined for the subject, as described hereinabove.

If the recommended treatment protocol is significantly different from the treatment protocol used in a previous round of treatment, at step 208 the recommended treatment protocol is provided by processor 110 to decision maker 118, which may be a human decision maker, such as a medical practitioner treating the subject, or may be an electronic decision maker, as described hereinabove.

As seen at step 210, input from the decision maker is received by processor 110. The decision maker may require that the recommended treatment protocol be modified prior to treating the subject, or may indicate that the recommended treatment protocol, or a similar treatment protocol based on the recommended treatment protocol, should not be used for the subject, and such requirements may be provided as the input. In embodiments in which the decision maker is human, the input may be provided via user interface 104 and input module 102.

At step 212, processor 110 checks whether or not the decision maker has approved treating the subject using a treatment protocol which is based on the recommended treatment protocol. If processor 110 identifies that the decision maker has not approved use of a treatment protocol based on the recommended protocol, the method returns to step 204 for selection of another recommended treatment protocol.

Otherwise, if processor 110 identifies that the decision maker approved use of the recommended treatment protocol, at step 214 the processor checks whether or not the decision maker has required making any changes to the recommended treatment protocol in order to obtain the treatment protocol to be used.

As seen at step 216, if such changes are required, processor 110 applies the changes to the recommended treatment protocol, thereby to generate the treatment protocol to be used. Once the treatment protocol is generated at step 216, the generated treatment protocol, or, in some embodiments, only the vibration protocol defined thereby, is provided by processor 110 to capsule control unit 116 at step 218.

Alternately, if no changes were required to the recommended treatment protocol (at step 214), or if the recommended treatment protocol is not significantly different from the treatment protocol used in an immediately previous round of treatment (at step 208), the treatment protocol to be used is set to be identical to the recommended treatment protocol, and the treatment protocol, or only the vibration protocol defined thereby, is provided by processor 110 to capsule control unit 116, at step 218.

As another alternative, the method may start from step 218, for example by providing to the capsule control unit 116 a default treatment protocol or a default vibration protocol to be used in a first treatment iteration. In such embodiments, the default treatment protocol or the default vibration protocol may be provided by the manufacturer of the capsule or by a medical practitioner for any subject beginning treatment using the system of FIG. 1, regardless of the subject's profile or of any data collected in the database regarding other subjects. In other embodiments, the method may begin with steps 200 and 202 so as to create the subject profile, and may then proceed directly to step 218 wherein the default treatment protocol or default vibration protocol is provided to the capsule control unit 116.

Once the capsule control unit 116 has received the vibration protocol to be used, possibly as part of the treatment protocol to be used, at step 220 the capsule control unit 116 programs a programmable vibrating ingestible capsule, such as capsule 114a or 114b, to implement the vibration protocol upon activation of the capsule or upon ingestion thereof, substantially as described hereinabove with respect to FIG. 1. In some embodiments, the capsule control unit 116 also provides output to the subject or to a caretaker thereof, for example via a display or other user interface, the output indicating the treatment procedures defined in the treatment protocol to be used.

As seen at step 222, once the programmable vibrating ingestible capsule (e.g. 114a or 114b) has been programmed to implement the vibration protocol, the capsule is activated, for example by the activation module of capsule control unit 116 as described hereinabove, and is provided to the subject for ingestion thereof, thereby to treat the subject. As explained hereinabove, the subject or a caretaker thereof is notified of the treatment procedures, and is instructed to ingest the programmed capsule in accordance with the treatment procedures defined in the treatment protocol being used.

In some embodiments, the capsule may be provided to the subject for ingestion without previous activation thereof. In such embodiments, the capsule may be activated while it is within the gastrointestinal tract of the subject, for example by providing a remote signal, from the exterior of the subject's body to a receiver of the capsule.

In some embodiments, the first vibrating ingestible capsule used to effect the first iteration of treatment is not a programmable capsule, but rather a programmed capsule, programmed to implement a specific vibration protocol, such as the default vibration protocol, for example as described hereinabove with reference to capsule 114a of FIG. 1. In some such embodiments, the method may begin at step 222, by activation of the programmed capsule and treatment of the subject therewith. In other embodiments, the method may begin with steps 200 and 202 so as to create the subject profile, and may then proceed directly to step 222 wherein the programmed capsule is activated and used to treat the subject.

During treatment of the subject, or following such treatment, processor 110 receives feedback from a user, which may be the subject, a medical practitioner treating the subject, and/or a caretaker of the subject, regarding the subject's response to the treatment, as seen at step 224. In some embodiments, the feedback is provided to processor 110 via user interface 104 and input module 102.

In some embodiments, the feedback may indicate times at which the subject experienced bowel movements during or following treatment in accordance with a specific treatment protocol, or a number of spontaneous bowel movements (SBM) or complete spontaneous bowel movements (CSBM) experienced during or following the treatment.

In some embodiments, the feedback may indicate a physical feeling experienced by the subject during or following treatment in accordance with a specific treatment protocol, such as a feeling of complete evacuation following a bowel movement, straining, bloating, gas, flatulence, pain during bowel movements, pain at times other than during a bowel movement, or other adverse events occurring during or following the treatment. For example, the subject may provide feedback indicating that vibration was felt when the capsule was passing through the rectal area of the subject, or that stomach pains were relieved following the treatment.

In some embodiments, the feedback may indicate treatment procedures used by the subject during implementation of the treatment protocol, such as, for example, a time of day at which a vibrating ingestible capsule was ingested.

In some embodiments, the feedback may indicate at least one characteristic of fecal matter excreted by the subject during or following the treatment in accordance with the treatment protocol. For example, the subject may provide feedback indicating the Bristol stool measure of a bowel movement following the treatment.

At step 226, the processor 110 may receive feedback from one or more sensors 120. In some embodiments, a sensor 120 is mounted in the toilet-bowl, and provides information regarding expelling of the vibrating ingestible capsule from the body of the subject. In some embodiments, the information includes an indication of a time at which the vibrating ingestible capsule was expelled from the body of the subject. In some embodiments, the information includes an identification of the expelled capsule. In some embodiments, the information includes information regarding fecal matter expelled by the subject, such as for example a Bristol stool measure of the fecal matter, as described hereinbelow.

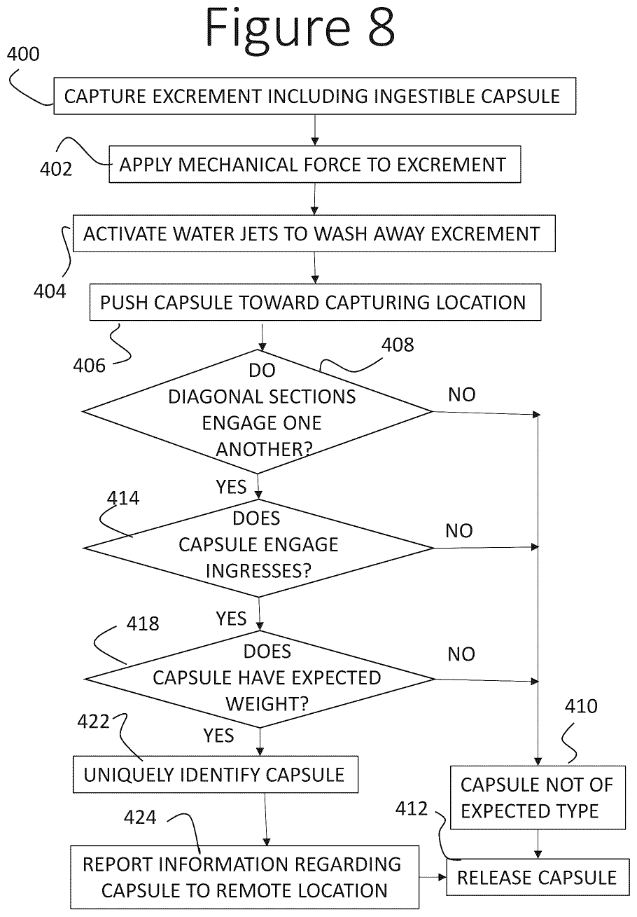

As discussed hereinabove with respect to FIG. 1, a toilet-bowl mounted sensor 120 may be any suitable sensor known in the art, such as that described in US Patent Application Publication No. 2009/0326514, or may be an inventive sensor as described hereinbelow with reference to FIGS. 3A to 7. An exemplary method by which the inventive sensors of FIGS. 3A to 7 obtain and provide feedback to processor 110 is described hereinbelow with respect to FIG. 8.

In some embodiments, at step 228 processor 110 may optionally compare, or cross-reference, the feedback provided by the user at step 224 to the feedback received from the sensor at step 226, and may assign a reliability weight to the feedback from the user and/or from the sensor based on the results of such comparison, as seen at step 230.

For example, the processor 110 may compare the times at which the subject reported to have had bowel movements, and the time at which the sensor reported that the capsule was expelled. If the subject did not report a bowel movement at the time that the capsule was expelled, this is indicative of the subject not providing accurate feedback, and as such the sensor based feedback would be assigned a higher reliability weight than the feedback received from the subject.

At step 232, the processor 110 updates the subject profile 106 based on the feedback provided by the subject, the medical practitioner, and/or the caretaker at step 224, and/or based on the feedback provided by the sensor 120 at step 226. In embodiments in which the subject and/or sensor feedback is assigned a reliability weight at step 230, the reliability weight is taken into consideration when updating the subject profile, such that a greater significance is given to the more reliable feedback.

Subsequently, the method returns to step 204, and an updated treatment protocol recommendation for the subject is obtained based on the updated user profile and based on data from database 108, which data relates to at least one other subject.

In some embodiments, following updating of the subject profile 106 at step 232, the processor 110 creates or updates an entry in database 108 relating to the subject, for example based on the update subject profile 106, as seen at step 234.

Reference is now made to FIGS. 3A, 3B, and 3C which are, respectively, a partially cut away side plan view, a partially cut away perspective view, and a top plan view of an inventive toilet-bowl mounted sensor 300 for capturing and identifying an ingestible capsule according to the present invention. In some embodiments, the toilet-bowl mounted sensor 300 may form part of the system of FIG. 1, for example as sensor 120.

As seen in FIGS. 3A-3C, sensor 300 includes a receptacle 302 surrounded at a top end thereof by a sealing rim 304. The sensor 300 is adapted to be placed within a toilet bowl 306, at a portion thereof in which sealing rim 304 engages, and seals against, the entire circumference of the toilet bowl, such that any excrement expelled into the toilet bowl would be caught in receptacle 302.

In some embodiments, the receptacle 302 is formed of a wire frame or a mesh frame, having gaps or openings large enough to allow water and excrement to pass through, but smaller than the diameter of the capsule to be caught in the receptacle, such that the capsule does not fall through the frame.

Extending along toilet bowl 306, from the exterior thereof to a controller 308 of sensor 300 (described in further detail hereinbelow), is at least one conduit 310 for providing water flow to sensor 300, and at least one wire 312 for providing electrical power to sensor 300 and/or for enabling communication of sensor 300 with a remote device, such as processor 110 of FIG. 1.

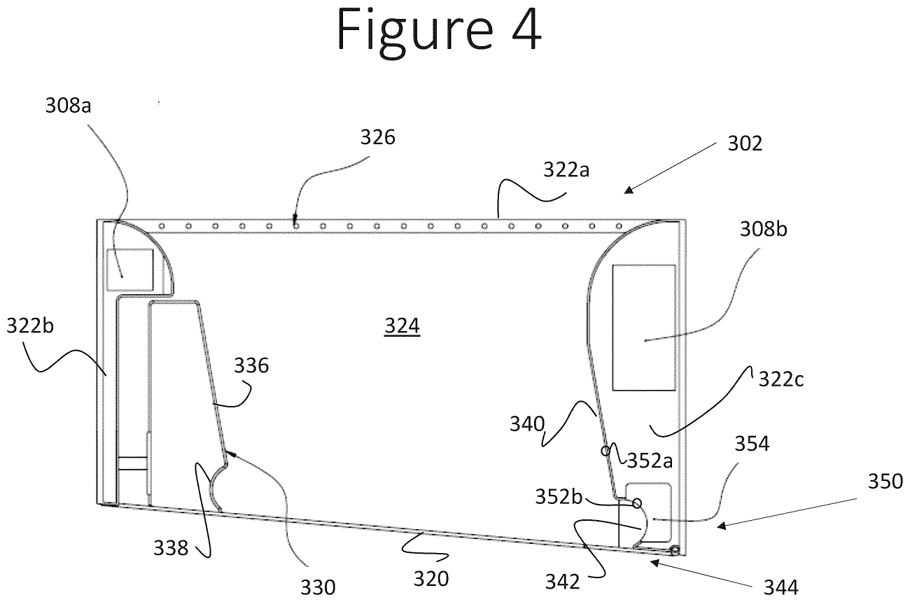

Reference is now made to FIG. 4, which is a sectional view of an embodiment of receptacle 302 of toilet-bowl mounted sensor 300 in a stand-by mode.

As seen, receptacle 302 has a wire frame bottom surface 320 as described hereinabove, and side walls 322 surrounding a hollow 324. At least one side wall, indicated in FIG. 4 by reference numeral 322a includes a plurality of water jets 326 which receive water from conduit 310 and are adapted to spray water into hollow 324, in order to wash out excrement therefrom.

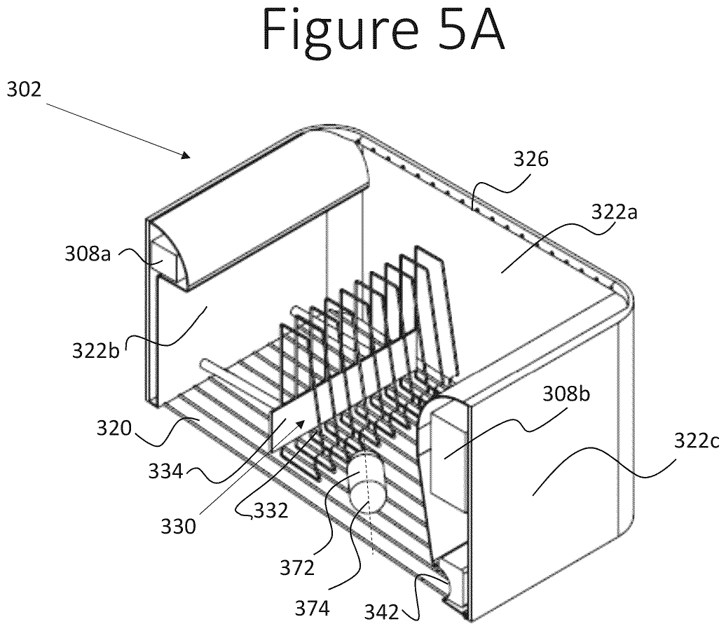

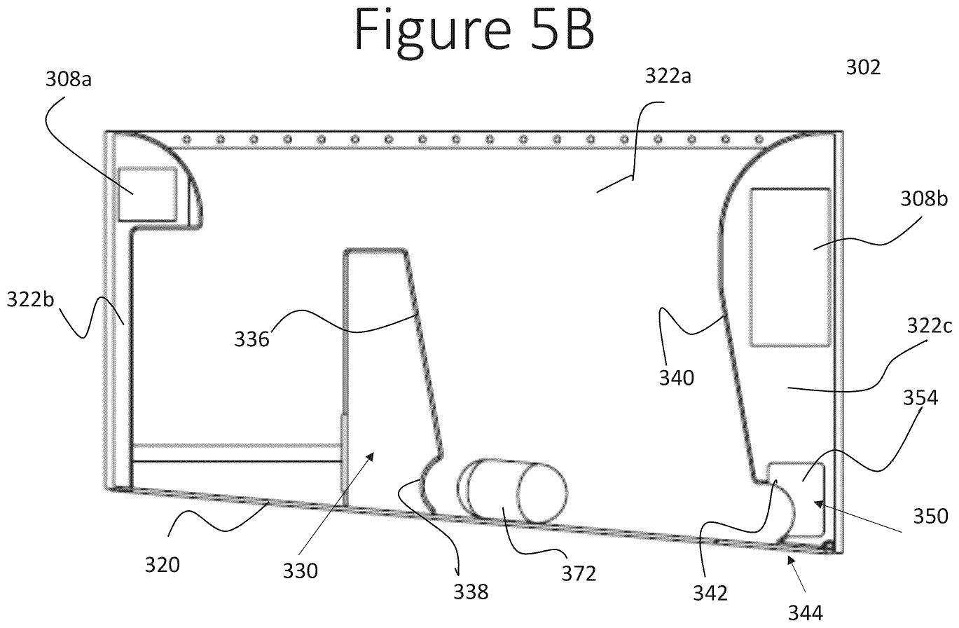

A pushing mechanism 330, which in some embodiments includes a plurality of wire frame elements 332 (seen clearly in FIG. 5A) connected to one another by a connector surface 334 (seen clearly in FIG. 5A), is disposed within receptacle 302 and is movable relative to one of side walls 322b, as explained in further detail hereinbelow. Pushing mechanism 330 is adapted to move back-and-forth along the receptacle 302 between side walls 322b and 322c, so as to provide mechanical force on excrement in the receptacle for breaking thereof into pieces that can be flushed out of the receptacle via the wire frame bottom 320. In some embodiments, pushing mechanism 330 is further adapted to sense the Bristol stool measure of the excrement in receptacle 302, for example by sensing the force exerted by the motor driving the pushing mechanism 330 for breaking down the excrement.

A side of pushing mechanism distal to the side wall 322b includes a diagonal section 336 terminating in a generally hemispherical ingress 338, which, together with a corresponding diagonal section 340 and generally hemispherical ingress 342 formed in an opposing side wall 322c of receptacle 302 are adapted for capturing a vibrating ingestible capsule and for measurement and identification thereof, as described in further detail hereinbelow.

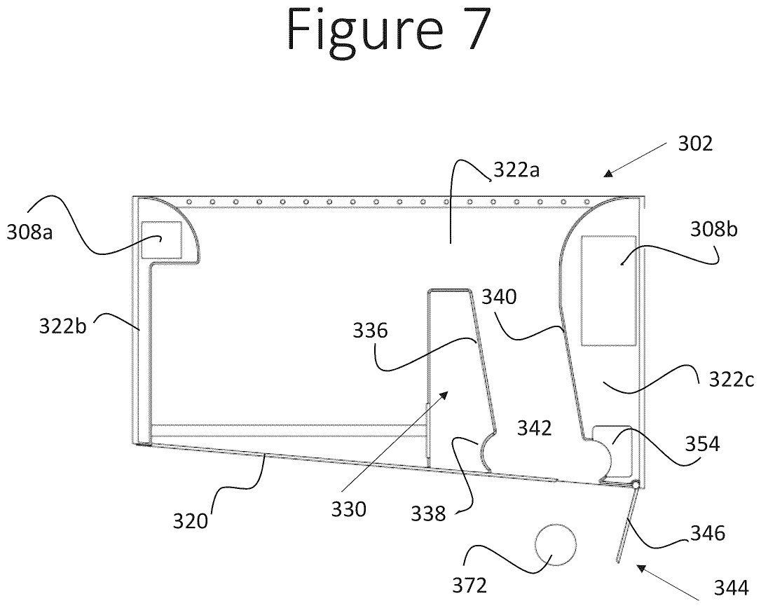

Disposed beneath ingress 342 is a capsule releasing mechanism 344, adapted, following capturing of an expelled capsule and identification thereof, to release the capsule from receptacle 302 for flushing thereof down the toilet. In some embodiments, releasing mechanism 344 comprises a hatch 346 (seen clearly in FIG. 7), hingedly connected to side wall 322c, which is adapted to open and release a captured capsule following identification thereof, and to close back immediately following removal of the capsule.

It will be appreciated that though in the illustrated embodiment the bottom surface 320 is slanted toward side wall 322c and ingress 342, so as to allow gravity to assist a capsule in arriving at its capturing location as described in further detail hereinbelow, in some embodiments the bottom surface 320 may be horizontal and perpendicular to the side walls 322b and 322c.

Included within side wall 322c, adjacent to ingress 342, is a capsule identification mechanism 350, which may include one or more sensors 352, a vision system 354, a communication system, and/or a weighing mechanism, for measuring the dimensions and/or weight of a captured capsule, and/or for uniquely identifying the capsule.

As described in further detail hereinbelow, sensors 352 may be contact sensors or distance sensors adapted to sense contact between, or a distance between, diagonal sections 336 and 340 and/or contact between a capsule captured between ingresses 338 and 342 and the ingress walls.

Vision system 354 has a field of view, and is typically adapted to view a wall of a captured capsule via the field of view, so as to uniquely identify the capsule. In some embodiments, the vision system may include a barcode reader adapted to identify a barcode printed onto the exterior of a captured capsule, a QR-code reader adapted to identify a QR-code printed onto the exterior of a captured capsule, an OCR mechanism adapted to scan and interpret text or an identification number printed onto the exterior of a captured capsule, or an image capturing and analysis mechanism adapted to capture an image of the exterior of the capsule and to analyze the captured image thereby to identify the capsule or a manufacturer thereof.

In some embodiments, the communication system includes an RFID tag reader adapted to read an RFID tag mounted on the exterior of a captured capsule, thereby to identify capsule. The communication system may be incorporated in the vision system or may be separate therefrom.

The weighing mechanism may be disposed on bottom surface 320 of the receptacle 302, preferably adjacent to ingress 342, so as to weigh a capsule captured between ingresses 338 and 342, as described in further detail hereinbelow. In some embodiments, weighing mechanism is mounted onto releasing mechanism 344.

Controller 308 of receptacle 302 may be disposed on or within one or more of side walls 322 and is adapted to control movement, and in some embodiments sensing capabilities, of pushing mechanism 330, and to control operation of identification mechanism 350 and of releasing mechanism 344. Additionally, controller 308 is adapted to provide power to pushing mechanism 330, releasing mechanism 344 and identification mechanism 350.

In some embodiments, controller 308 is adapted to receive input from weighing mechanism 344 and/or from identification mechanism 350, so as to identify that a capsule has been captured, and the dimensions, weight, and/or identity of the captured capsule. In some embodiments, controller 308 includes a clock for identifying a time at which a capsule was captured and/or identified.

Typically, controller 308 is adapted to communicate with a remote location, such as processor 110, via a communication module forming part of the controller and/or via a communication wire 310 extending from receptacle 302, along toilet bowl 306 to the remote location. In some embodiments, the controller 308 may communicate the time at which the capsule was captured, and information regarding the capsule, such as its dimensions, weight, and/or identity, to the remote location, for use thereof. For example, in the embodiment of FIGS. 1 and 2, the information provided by the controller 308 may be used to identify a time that the capsule has spent in the GI tract of the subject, or an order at which a plurality of ingested capsules were expelled.

In some embodiments, the controller 308 may further communicate information relating to characteristics of the expelled fecal matter, such as a Bristol stool measure thereof, as sensed by pushing mechanism 330 or by the motor driving the pushing mechanism. In some such embodiments, the controller 308 only communicates the information relating to the fecal matter if a capsule was captured, or if a capsule of a specific type was captured.

In the embodiment illustrated in FIG. 4, controller 308 has a first portion 308a disposed within side wall 322b and adapted to control and to provide power to pushing mechanism 330, and a second portion 308b disposed within side wall 322c and adapted to control and to provide power to releasing mechanism 344 and identification mechanism 350. However, any other arrangement of controller 308 is considered within the scope of the invention.

Reference is now made to FIGS. 5A and 5B, which are, respectively, a perspective view and a sectional view of the receptacle 302, in an operational mode. As seen, pushing mechanism 330 is being pushed away from side wall 322b and toward side wall 322c, as indicated by arrow 370. A vibrating ingestible capsule 372, which was expelled from the body of a subject together with fecal matter thereof, is caught by receptacle 302, and may be pushed by pushing mechanism 330 toward ingress 342 and identification mechanism 350. In some embodiments, capsule 372 is generally ovoid, is symmetrical about a longitudinal axis 374, and has a generally circular cross-section in a direction perpendicular to axis 374.

It will be appreciated that pushing mechanism 330 may be pushed in the direction of arrow 370 and back in the opposite direction multiple times, so as to break down fecal matter caught in receptacle 302 for removal thereof via bottom surface 320. In some embodiments, motion of pushing mechanism 330 is accompanied by water spray from jets 326 for washing away fecal matter and cleaning of the capsule, prior to pushing and/or capturing thereof.

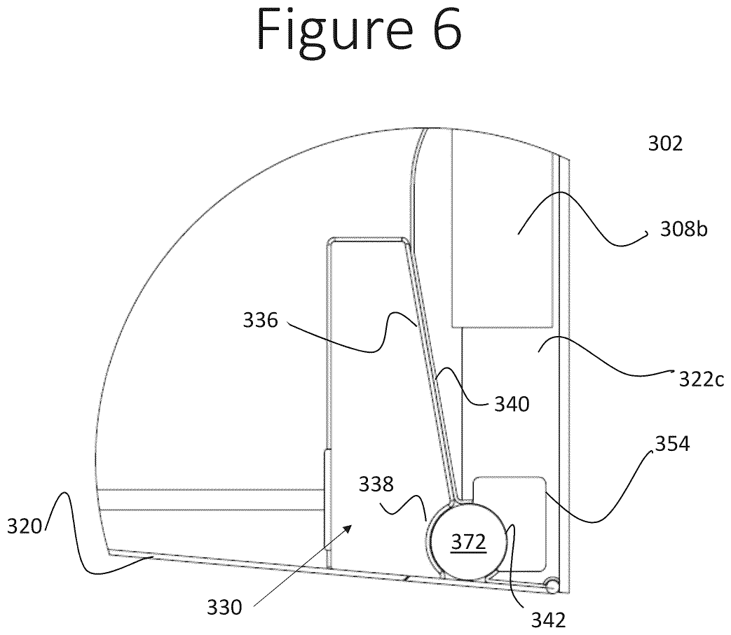

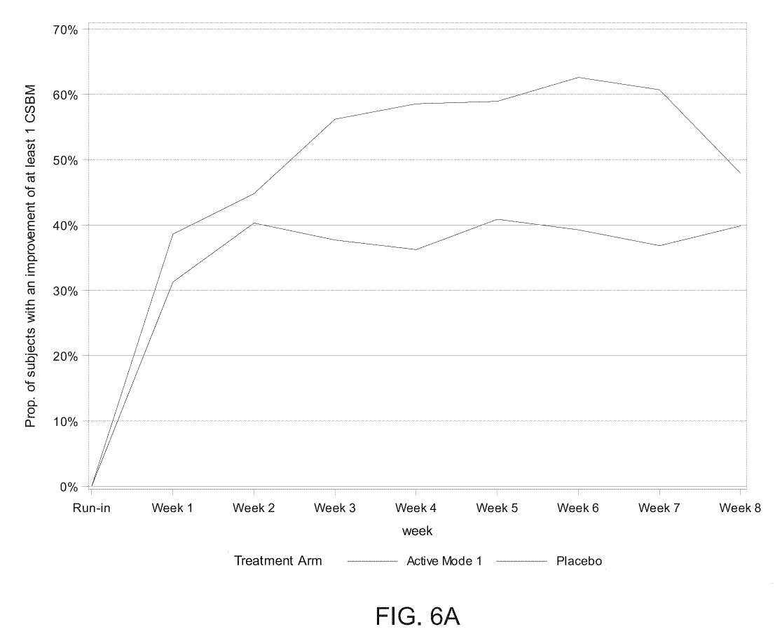

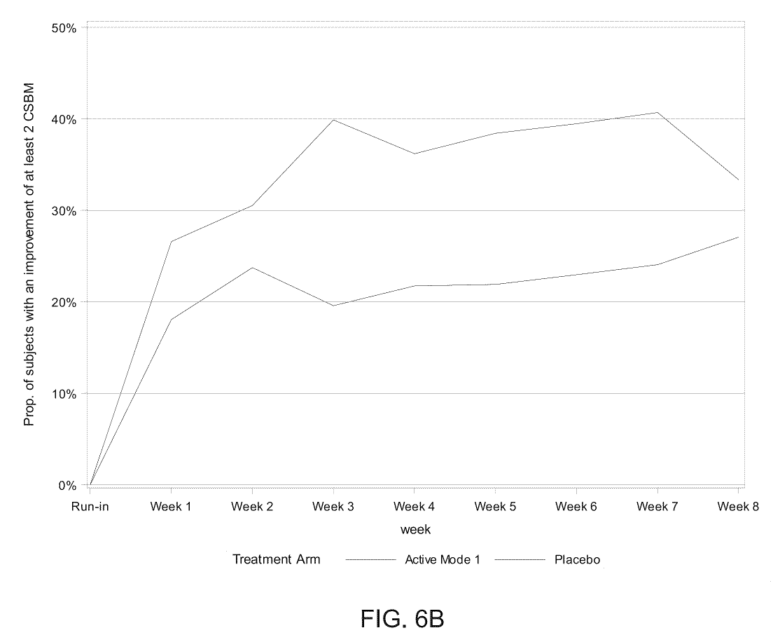

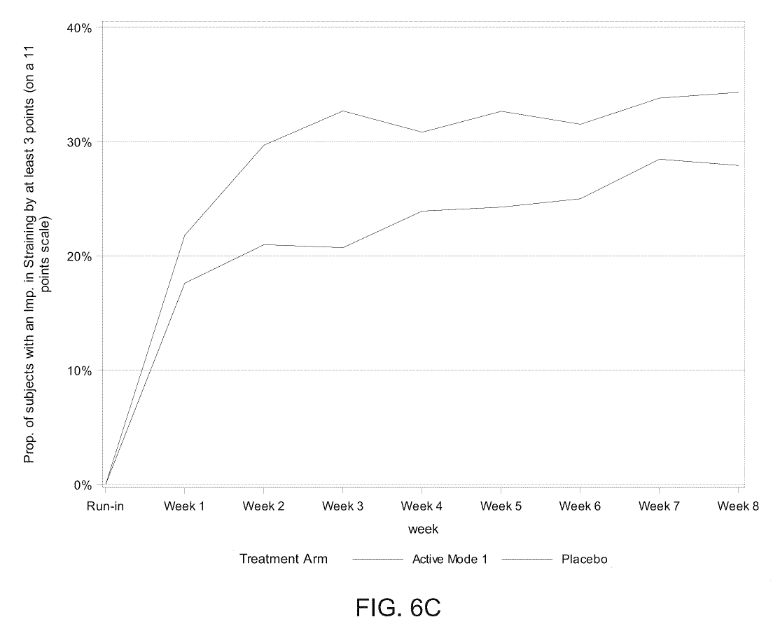

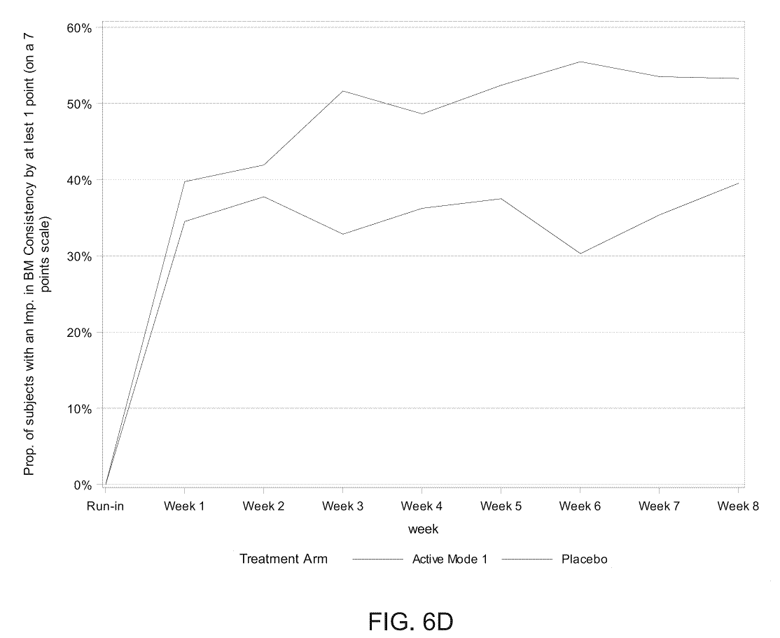

Reference is now made to FIG. 6, which is a partial sectional view of receptacle 302 in a capsule capturing mode. As seen in FIG. 6, pushing mechanism 330 is pushed distally from side wall 322b until it engages side wall 322c, such that diagonal sections 336 and 340 engage one another, and such that the capsule 372 is trapped between ingresses 338 and 342. It is a particular feature of the present invention that the arrangement of the wire frame of bottom surface 320, and ingress 338 of pushing mechanism 330, cause capsule 372 to be captured such that longitudinal axis 374 is generally parallel to side wall 322c, and such that the circular cross section of the capsule substantially fills the circular cross-section formed by ingresses 338 and 342.

It is a particular feature of the teachings herein that capturing of the capsule, results in measurement of its dimensions. Specifically, sensors 352 of the identification mechanism 350 identify the dimensions of the capsule. Sensors 352a mounted along section 340 identify whether or not diagonal sections 336 and 340 engage one another—if the diagonal sections do not engage one another when the capsule is captured, the capsule has a greater circumference than that of the circular cross section of the cavity between ingresses 338 and 342. Sensors 352b mounted along ingress 342 identify whether or not the circumference of the capsule engages the ingress wall—if the circumference of the capsule does not engage the ingress walls when the capsule is captured, the capsule has a smaller circumference than that of the circular cross section of the cavity between ingresses 338 and 342. In some embodiments, weighing mechanism 358, which may be disposed beneath ingresses 338 and 342, identifies the weight of capsule 372 while the capsule is captured.

In some embodiments, the circular cross-section of the cavity between ingresses 338 and 342 may be specifically sized to match the cross section of a particular type of capsule, such that controller 308 may identify, based on input received from sensors 352, whether or not the captured capsule is of the particular type. Additionally, even if the dimensions of captured capsule 372 match the expected dimensions, controller 308 may use the identified weight of the capsule to determine whether the capsule is indeed of the expected particular type. The controller 308 may report capture of the capsule, for example to processor 110, if the capsule has the expected dimensions and weight, and may choose not to report capture of the capsule if it is differently sized or has a different weight.

When the capsule 372 is captured between ingresses 338 and 342, vision system 354 may view a portion of the exterior of the capsule 372 via field of view 355, and may thus uniquely identify the capsule. As described above, in one example, the capsule may have a barcode printed thereon, and the vision system includes a barcode reader adapted to identify the capsule by reading the barcode printed thereon. In another example, the capsule may have a QR-code printed thereon, and the visions system includes a QR-code reader adapted to identify the capsule by reading the code printed thereon. In yet another example, the capsule has a specific logo and/or an identification or serial number printed on the exterior thereof, and vision system 354 includes an OCR mechanism or other mechanism suitable for reading the logo and serial number, thereby to identify the capsule.

In some embodiments, identification of the capsule may alternately or additionally be accomplished by communication system 356. For example, the communication system may include an RFID tag reader, which may identify an RFID tag mounted on the exterior of the captured capsule. Following identification of the capsule, the controller 308 may report capturing of the capsule, and information about the capsule, to a remote location, as described hereinabove. In some embodiments, the controller 308 may report the information only if the capsule is identified as belonging to a specific manufacturer, or as being of a specific type, for example as described hereinabove.

Reference is now made to FIG. 7, which is a sectional view of receptacle 302 in a capsule releasing mode. As seen in FIG. 7, following completion of the capsule identification, pushing mechanism 330 may be displaced away from side wall 322c so as to release the capsule 372 from the hollow between ingresses 338 and 342, and hatch 346 of releasing mechanism 344 may open, for example by rotating downwardly relative to bottom surface 320, so as to allow the capsule 372 to drop out of receptacle 302b, and to be flushed down the toilet. In some embodiments, the releasing mechanism is electrically controlled by controller 308, such that hatch 346 recloses after a predetermined duration during which it is assumed that the capsule has been removed from the receptacle, or after sensors, for example on hatch 346, identify releasing of the capsule.

Reference is now additionally made to FIG. 8, which is a schematic flowchart of a method for capturing and identifying an ingestible capsule that has been expelled into a toilet bowl according to the present invention, the method utilizing the inventive toilet-bowl mounted sensor 300 of any one of FIGS. 3A-7.

At an initial step 400, excrement including an ingestible capsule is captured in a receptacle, such as receptacle 302, of the sensor 300. The pushing mechanism 330 is pushed forward (toward side wall 322c) and back (away from side wall 322c and toward side wall 322b), so as to apply mechanical force to the excrement for breaking down thereof, as seen at step 402.

Subsequently or simultaneously to the breaking action of the pushing mechanism, at step 404, water jets 326 are activated to wash away excrement, which may be removed from the receptacle via the wire frame of bottom surface 320.

In some embodiments, steps 402 and 404 may be repeated for a predetermined number of cycles, a predetermined duration, or until controller 308 determines that there is little or no residual excrement remaining on the capsule or in the receptacle.

At step 406, following removal of excrement and cleaning of the capsule, the pushing mechanism 330 is displaced toward side wall 322c, pushing the capsule in that direction, until the pushing mechanism 330 can move no further.

At step 408, controller 308 assesses whether or not the pushing mechanism has engaged the side wall of the receptacle, and more specifically, whether or not diagonal sections 336 and 340 engage one another, for example using sensors 352a (FIG. 7).

If the pushing mechanism can move no further, but diagonal sections 336 and 340 do not engage one another, this is indicative of the captured capsule having a greater cross-section circumference than the expected capsule, and at step 410 the controller concludes that the captured capsule is not of the expected type. At step 412, the releasing mechanism is operated to release the captured capsule, for example by opening hatch 346 as described hereinabove.

Otherwise, if at step 408 it is determined that the diagonal sections 336 and 340 engage one another, at step 414 controller 308 assesses whether the exterior surface of the captured capsule engages the surface of the cavity between ingresses 338 and 342, for example by using sensor 352b (FIG. 7).

If the capsule does not engage the surface of the cavity when diagonal sections 336 and 340 engage one another, this is indicative of the captured capsule having a smaller cross-section circumference than the expected capsule, and the method continues to step 410 where the controller concludes that the captured capsule is not of the expected type and to step 412 where the capsule is released from the receptacle.

If the capsule is determined to have the expected dimensions, controller 308 may receive from the weighing mechanism information relating to a weight of the captured capsule, and may determine, at step 418, whether or not the measured weight of the capsule matches, or is within a predetermined margin of error, of an expected weight of the capsule. If the measured weight of the capsule does not match the expected weight, the method continues to step 410 where the controller concludes that the captured capsule is not of the expected type and to step 412 where the capsule is released from the receptacle.

Otherwise, the controller concludes that the capsule is of the expected type, and at step 422 the identification mechanism uniquely identifies the capsule, for example by reading a barcode, QR code, unique identification number or string of characters, or an RFID tag located on the exterior surface of the capsule.

At step 424 the controller may report to a remote location, such as processor 110 of FIG. 1, information relating to the capsule, such as the time the capsule was expelled from the body of the user or the time that the capsule was identified by the identification mechanism, and/or the unique identification of the capsule, which information may be used to better understand the subject's response to treatment with the capsule. In some embodiments, the controller may further report information regarding the fecal matter, such as a Bristol stool measure thereof, as identified by the pushing mechanism and as described hereinabove. As discussed in detail hereinabove, the reporting may be carried out by wired or wireless communication between the controller 308, or another portion of the sensor 300, and the remote location.

Subsequently or in parallel to such reporting, the capsule is released from the receptacle, at step 412.

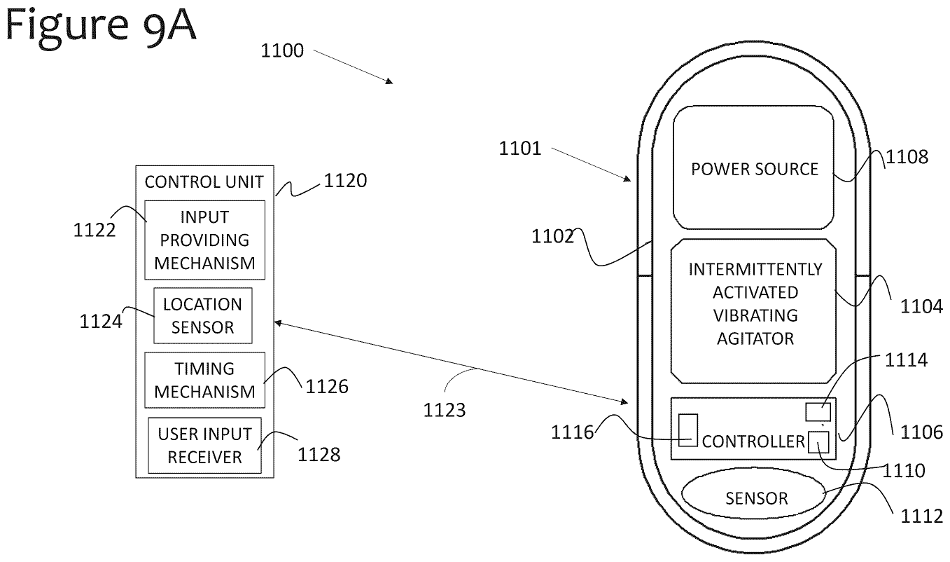

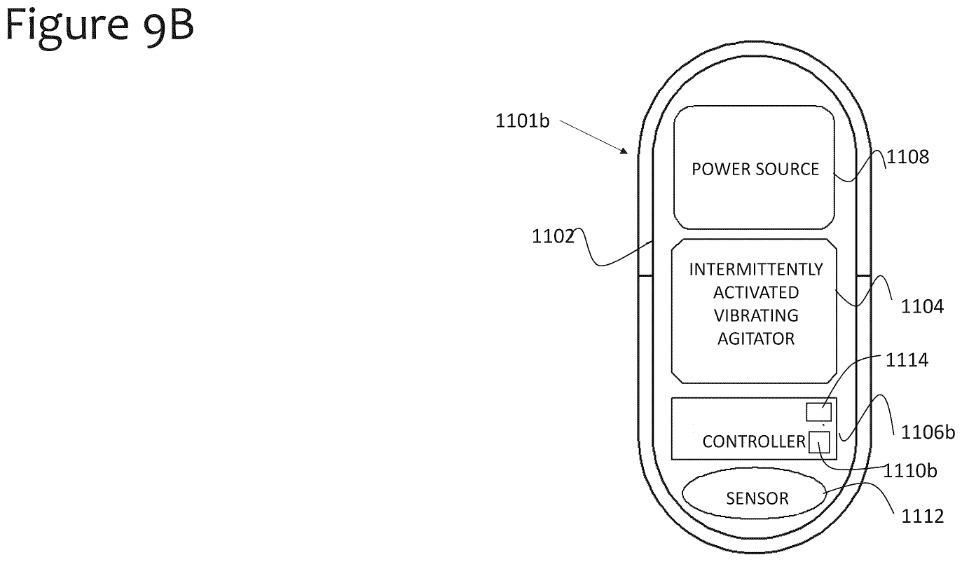

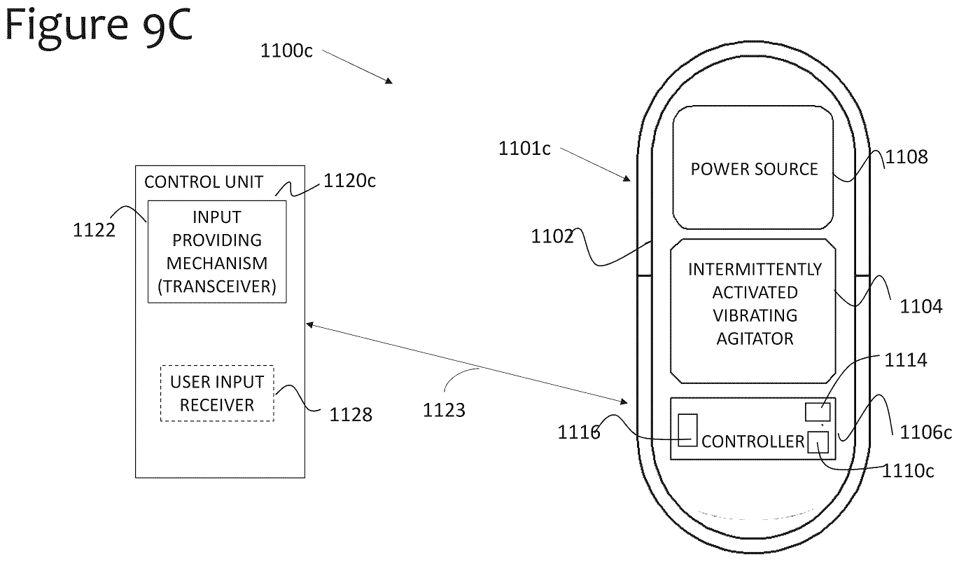

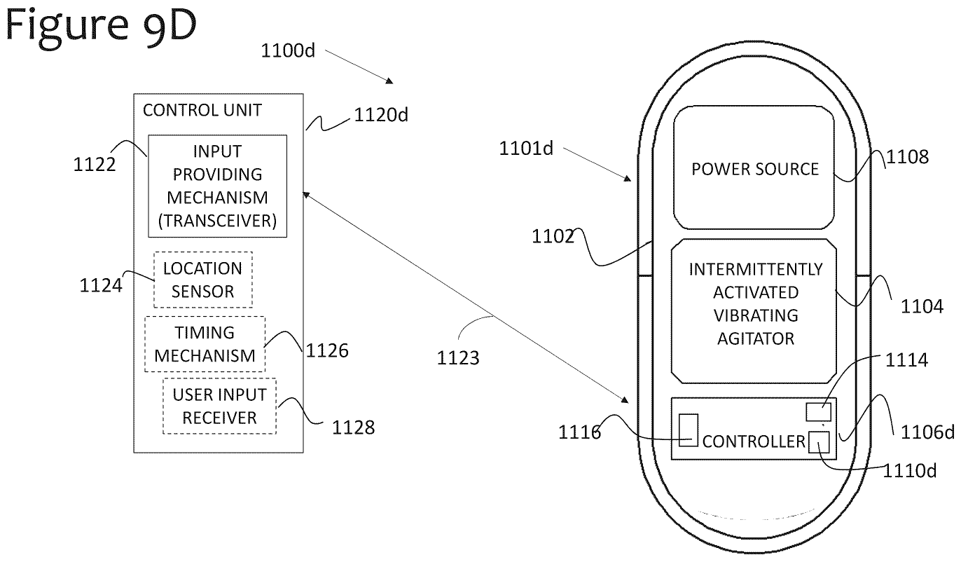

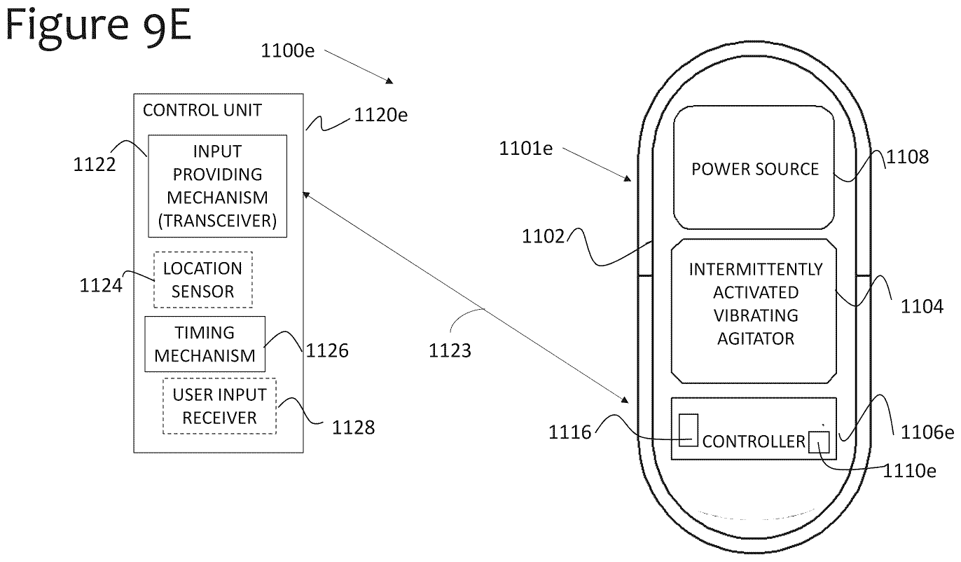

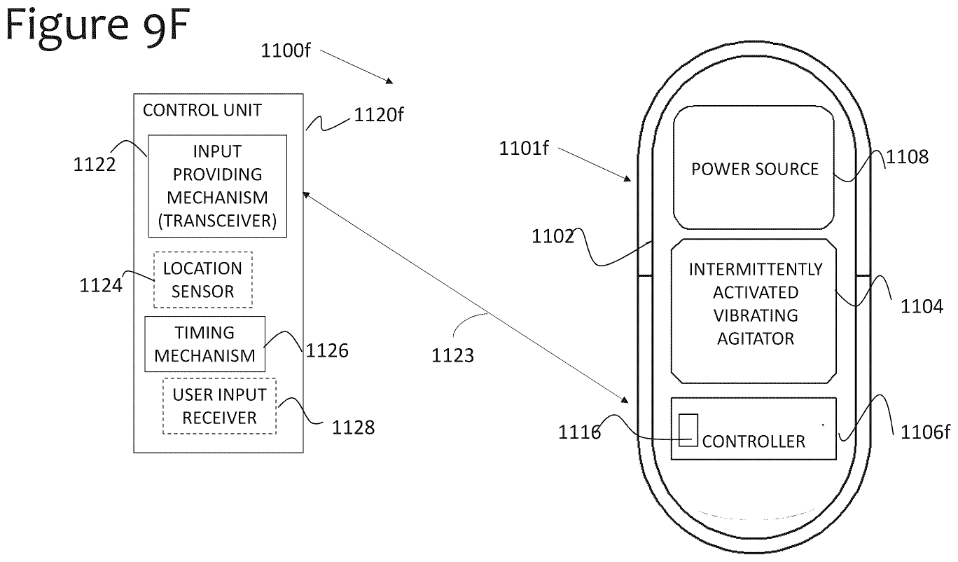

Reference is now made to FIGS. 9A to 9F, which are schematic block diagrams of various embodiments of gastrointestinal treatment systems including vibrating ingestible capsules according to embodiments of the present invention.

The gastrointestinal treatment systems of FIG. 9A to 9F may be, or may form part of, gastrointestinal treatment system 100 described hereinabove with respect to FIG. 1. For example, the vibrating ingestible capsules of FIGS. 9A to 9F may be equivalent to, or may implement, vibrating ingestible capsule 114a and/or 114b described hereinabove with respect to FIG. 1. As another example, the capsule control units of FIGS. 9A to 9F may be equivalent to, or may implement, control unit 116 of FIG. 1. As such, vibrating ingestible capsules of FIGS. 9A to 9F may be associated with a computer readable memory storing a user profile and/or a database, substantially as described hereinabove with respect to FIG. 1.

As seen in FIG. 9A, a general gastrointestinal treatment system 1100 includes vibrating ingestible capsule 1101. Capsule 1101 includes a capsule housing or shell 1102, arranged along a longitudinal axis and having disposed therein a vibrating agitator 1104. A controller 1106, which may for example be, or include, a processor, is adapted to control operation of the vibrating agitator 1104, and at least one power source 1108 provides power to vibrating agitator 1104 and controller 1106.

Power source 1108 may be any suitable power source, such as one or more alkaline or silver oxide batteries, primary batteries, rechargeable batteries, capacitors and/or supercapacitors.

Intermittently activated vibrating agitator 1104 is adapted to have a vibration mode of operation (also termed the first mode of operation) and a rest mode of operation (also termed the second mode of operation). In the vibration mode of operation, intermittently activated vibrating agitator 1104 is adapted to exert forces on capsule housing 1102, such that capsule housing 1102 exerts vibrations on an environment surrounding capsule 1101.

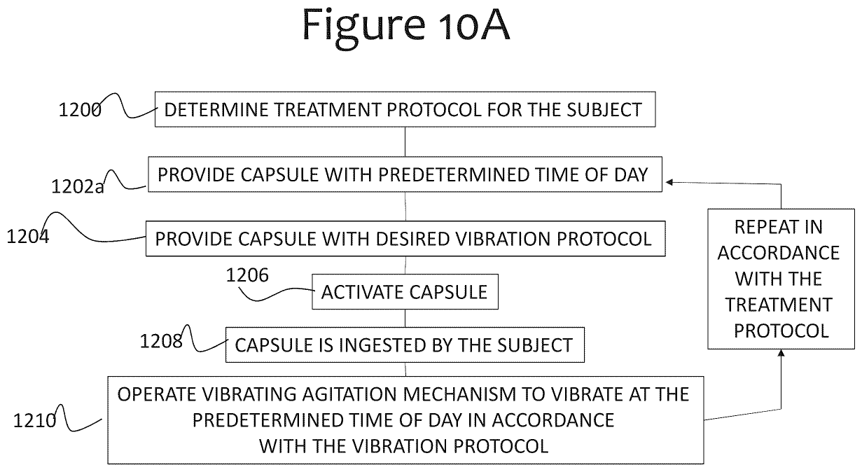

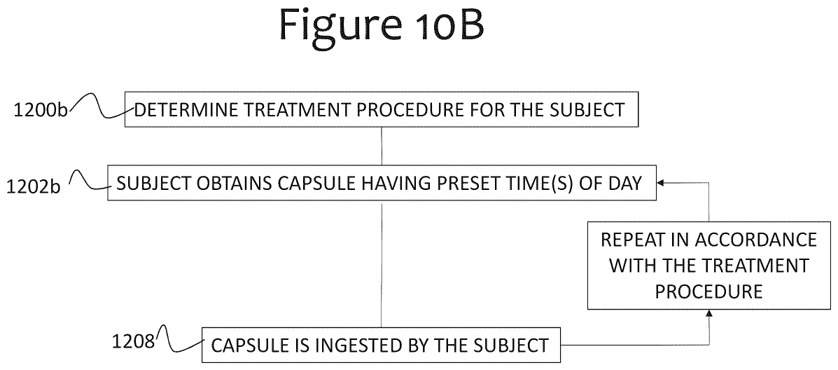

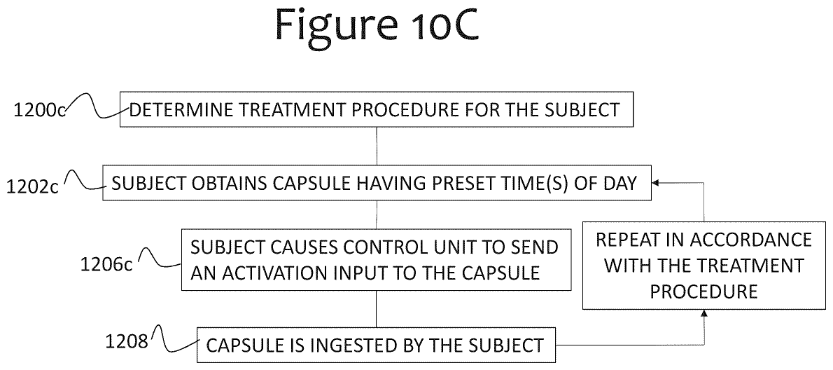

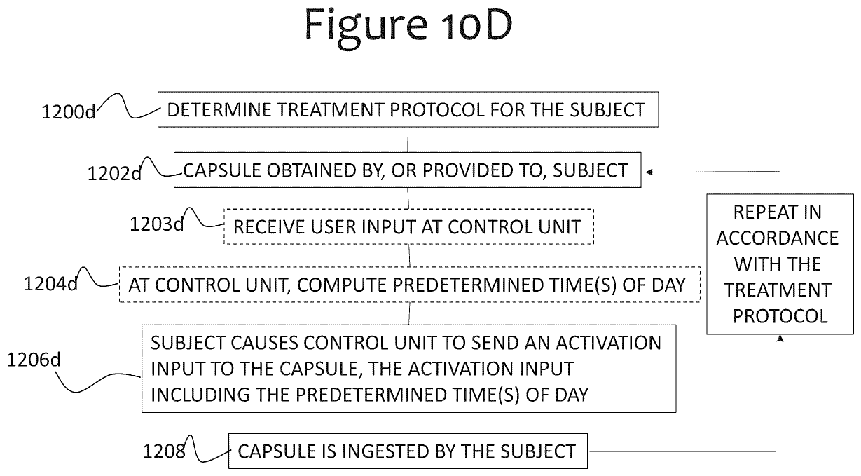

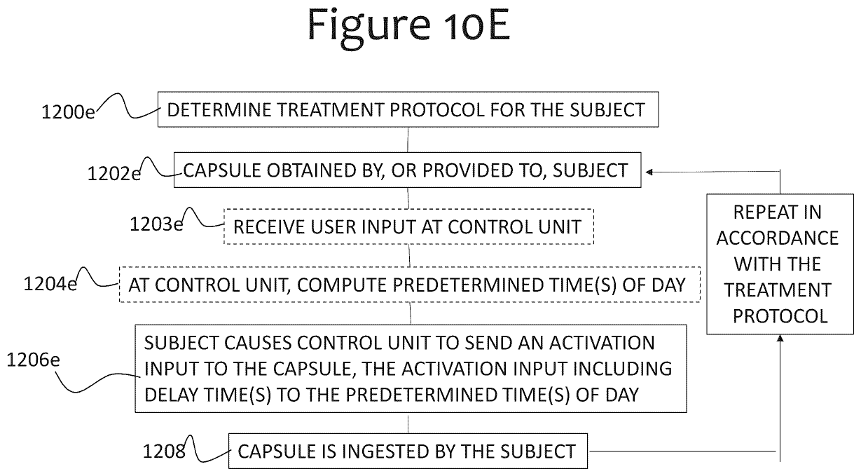

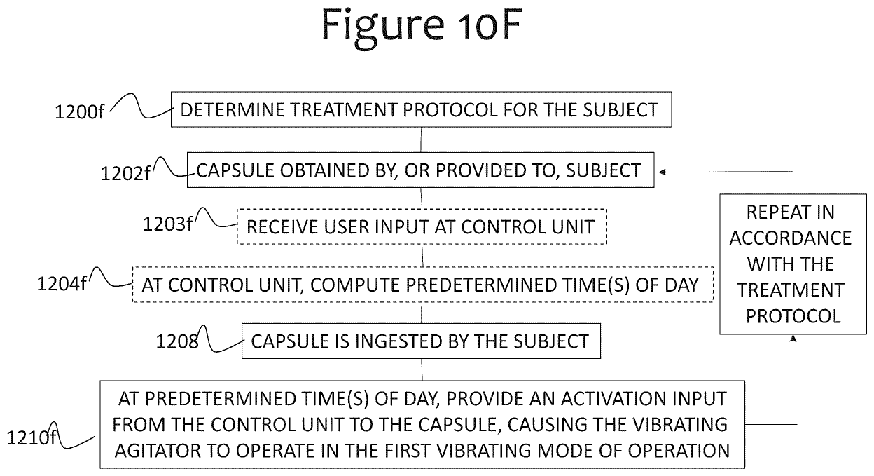

It is a particular feature of the present invention that controller 1106 is adapted, in response to receipt of an activation input or when the capsule is in an operative state, to activate vibrating agitator 1104 to operate in the vibrating mode of operation at at least one predetermined time of day, as described in detail hereinbelow with respect to FIGS. 10A to 10F.

In some embodiments, controller 1106 is adapted to activate vibrating agitator 1104 to operate in the vibrating mode of operation at the at least one predetermined time of day, on at least two consecutive days. For example, if controller 1106 is adapted to activate vibrating agitator 1104 to operate in the vibrating mode of operation at 1:00 pm, and the capsule is ingested by the subject at 6:00 pm on Sunday, controller 1106 will activate vibrating agitator 1104 at 1:00 pm on Monday, and again at 1:00 pm on Tuesday. In such embodiments, each activation of the vibrating agitator may be for a predetermined duration, e.g. 2 hours. In such embodiments, power source 1108 carries sufficient charge for activation of the vibrating agitator on at least two consecutive days.

In some embodiments, the capsule is in an inoperative state, until the receipt of an activation input, which causes controller 1106 to transition the capsule from the inoperative state to an operative state.

In some embodiments, controller 1106 is functionally associated with, or includes, a timing mechanism 1110, powered by power source 1108 and adapted to track at least one time characteristic. In some embodiments, the timing mechanism 1110 comprises a clock. In some embodiments, the timing mechanism 1110 comprises a timer, for example adapted to track a duration that has passed since an activation input was received, or a duration that has passed since the subject ingested capsule 1101.

In some embodiments, in response to receipt of an activation input, controller 1106 is adapted to activate operation of a clock timing mechanism 1110 to track a time of day, so as to identify the at least one predetermined time of day for activation of vibration agitator 1104.

In some embodiments, capsule 1101 is devoid of any sensors for sensing an environment thereof, and includes, as timing mechanism 1110, a timer. In some such embodiments, controller 1106 is adapted, in response to receipt of an activation input, to wait a predetermined delay time, or a delay time provided as part of the activation input, and following the delay time, at a time coinciding with the at least one predetermined time of day, to activate vibrating agitator 1104 to operate in the first vibration mode of operation.

In other embodiments, such as the embodiment illustrated in FIG. 9A, capsule 1101 further includes at least one sensor 1112, functionally associated with controller 1106. The at least one sensor 1112 may be adapted to sense at least one parameter within capsule 1101 or in an environment of capsule 1101, and may include a temperature sensor, an illumination sensor, a moisture sensor, a pressure sensor, an accelerometer, or any other suitable sensor. In some embodiments, the at least one sensor 1112 is adapted to identify a specific condition in capsule 1101 or in the vicinity thereof, and to provide an activation input to controller 1106 in response to identification of the condition. For example, in some embodiments the condition is indicative of the subject ingesting capsule 1101. For example, in some embodiments sensor 1112 may include an illumination sensor, adapted to identify transition of capsule 1101 from an illuminated environment (e.g. outside the human body) to a dark environment (e.g. within the human body) and to provide an activation input in response to identification of such a transition.

As another example, in some embodiments sensor 1112 may include a pressure sensor adapted identify pressure applied to the capsule 1101, which pressure is indicative of the capsule moving through a pharynx of the subject, and to provide an activation input in response to identification of such pressure.

As a further example, in some embodiments sensor 1112 may include a temperature sensor adapted to identify transition of capsule 1101 from an area with ambient temperature (e.g. outside the human body) to an area with a human body temperature and to provide an activation input in response to identification of such a transition.

As another example, in some embodiments, sensor 1112 may include a motion or acceleration sensor, such as an accelerometer, adapted to identify an activation motion carried out by a user on capsule 1101 and to provide an activation input in response to identification of such a transition. An example of an accelerometer providing an activation input for a gastrointestinal capsule is provided in U.S. patent application Ser. No. 15/168,065 filed on May 29, 2016 (and published as US 2017/0340242), which is incorporated by reference for all purposes as if fully set forth herein.

As a further example, in some embodiments sensor 1112 may include a moisture sensor adapted to identify transition of capsule 1101 from a dry area (e.g. outside the human body) to a moist area (e.g. within the human body) and to provide an activation input in response to identification of such a transition.

In some embodiments, such as the embodiment illustrated in FIG. 9A, capsule 1101 further includes a location sensor 1114, such as a GPS or GLONASS receiver, functionally associated with controller 1106. Location sensor 1114 may be adapted to identify the geographic location of the capsule.

In some embodiments, system 1100 further includes a control unit 1120, which is typically remote from capsule 1101, and which is adapted to provide one or more inputs to the capsule. In some such embodiments, capsule 1101 further includes a remote input receiving mechanism 1116, such as a transceiver, functionally associated with controller 1106, and adapted to receive inputs from an input providing mechanism 1122 of control unit 1120.

In some embodiments, control unit 1120 may further include a location sensor 1124, such as a GPS or GLONASS receiver, adapted to identify the geographic location of the control unit.

In some embodiments, control unit 1120 may further include a timing mechanism 1126, adapted to track at least one time characteristic, such as the time of day, or a duration that has passed since a control instruction was provided to capsule 1101. Typically, the timing mechanism 1126 comprises a clock.

In some embodiments, control unit 1120 may further include a user input receiver 1128, such as a keyboard, touch screen, or touch pad, adapted to receive input from a user, such as the subject, a medical professional treating the subject, or a caregiver of the subject.

Control unit 1120 may be any suitable type of control unit. In some embodiments, control unit may be a suitably configured smart phone or a tablet computer.

In some such embodiments, control unit 1120 may provide inputs to capsule 1101 by remotely transmitting the inputs from input providing mechanism 1122 to remote input receiving mechanism 1116, for example using a short-range wireless communication method indicated by arrow 1123, such as radio frequency (RF) communication or Bluetooth® communication. One example of such a mechanism for providing input to a capsule is described in U.S. patent application Ser. No. 15/132,039 filed Apr. 18, 2016 (and published as US2017/0296428), and entitled “IN VIVO DEVICE AND METHOD OF USING THE SAME”, which is incorporated by reference for all purposes as if fully set forth herein.

In some embodiments, control unit 1120 is adapted to provide the activation input to controller 1106 of capsule 1101. In some such embodiments, control unit 1120 provides the activation input prior to the subject ingesting capsule 1101, whereas in other embodiments control unit 1120 provides the activation input following ingestion of capsule 1101 by the subject.

As discussed hereinabove, controller 1106 of capsule 1101 is adapted to activate vibrating agitator 1104 to operate in the vibrating mode of operation at at least one specific time of day. That specific time of day may be identified and/or provided to controller 1106 by any of a number of methods or mechanism, some exemplary ones of which are described herein.

In some embodiments, controller 1106 of capsule 1101 is pre-programmed with the predetermined time of day, which may be, for example, a default time of day. In such embodiments, timing mechanism 1110 of capsule 1101 comprises a clock, adapted to identify the predetermined time of day.

In some embodiments, capsule 1101 is adapted to be used to mitigate jetlag of a user travelling from an origin location having an origin time zone to a destination location having a destination time zone. In some such embodiments, the pre-determined time of day is a time of day in the origin time zone. In other such embodiments, the pre-determined time of day is a time of day in the destination time zone. In yet other embodiments, the predetermined time of day is a time of day in a home time zone, which is the time zone at which the subject normally resides, or has been residing for at least a predetermined duration, for example at least one week.

In some such embodiments, timing mechanism 1110 provides controller 1106 with the current time of day, such that, following receipt of the activation input, or once capsule 1101 is in the operative state, controller 1106 tracks the current time of day, for example in the origin time zone, the destination time zone, or the home time zone, until arrival of the predetermined time, and then activates vibrating agitator according to a suitable vibration protocol.

In some embodiments, the predetermined time of day is provided to controller 1106 as part of the activation input, or in another input, for example provided by control unit 1120.

In some embodiments, regardless of whether the predetermined time of day is pre-programmed or is provided to controller 1106 as an input, the activation input (or another input) provided to controller 1106, for example by control unit 1120, includes the current time of day at the time of providing the activation input, for example in the origin time zone, the destination time zone, or the home time zone. In such embodiments, controller 1106 and/or timing mechanism 1110 is adapted to compute a delay time from the received current time of day (e.g. the time of the activation input) to the predetermined time of day, to wait the computed delay time and subsequently to activate vibrating agitator 1104 according to a suitable vibration protocol.

In another embodiment, controller 1106 may or may not be provided with the predetermined time of day. However, at the time of providing the activation input from control unit 1120, using timing mechanism 1126, control unit 1120 computes a time delay between the current time and the predetermined time of day. Control unit 1120 provides an activation input to controller 1106, indicating the computed time delay that controller 1106 should wait prior to activation of vibrating agitator 1104, so that vibrating agitator 1104 will operate in the vibrating mode of operation at the predetermined time of day. Using timing mechanism 1110, controller 1106 waits the computed time delay prior to activation of vibrating agitator, such that the first mode of operation of vibrating agitator 1104 occurs at the predetermined time of day. In some such embodiments, timing mechanism 1110 includes a timer.

In yet another embodiment, at the predetermined time of day, as indicated by timing mechanism 1126, control unit 1120 identifies that the current time of day is the predetermined time, and provides an activation input to controller 1106 indicating that the vibrating agitator should be activated at this time. Controller 1106 is adapted to activate vibrating agitator 1104 to operate in the vibrating mode of operation immediately responsive to receipt of such an input. In some such embodiments, capsule 1101 is devoid of a timing mechanism 1110.

In some embodiments, the predetermined time of day is, or coincides with, at least one predetermined mealtime.

In some such embodiments, the predetermined mealtime is a default mealtime. For example, the default mealtime may be a default breakfast time, which may be between 5 am and 10 am, between 6 am and 10 am, between 6 am and 9 am, between 6 am and 8 am, between 7 am and 10 am, between 7 am and 9 am, and between 7 am and 8 am. As another example, the default mealtime may be a default lunch time, which may be between 12 pm and 3 pm, between 12 pm and 2 pm, or between fpm and 3 pm. As yet another example, the default meal time may be a default supper time, which may be between 6 pm and 10 pm, between 7 pm and 10 pm, between Bpm and 10 pm, between 6 pm and 9 pm, between 7 pm and 9 pm, or between 6 pm and 8 pm.

In some embodiments, the at least one predetermined time of day includes at least two predetermined times of day. In some embodiments, the at least two predetermined times of day include lunchtime. In some embodiments, the at least two predetermined times of day include suppertime. In some embodiments, the at least two predetermined times of day include at least two mealtimes. In some embodiments, the at least two mealtimes include lunchtime and suppertime.

In some embodiments, the vibrating agitator is activated at the at least two mealtimes on at least two consecutive days. For example, the vibrating agitator may be activated at lunchtime and suppertime on Monday, and then again at lunchtime and suppertime on Tuesday.

In some embodiments, the vibrating agitator may also be activated an additional time, at one of the predetermined mealtimes, on a third consecutive day. For example, the vibrating agitator may be activated at lunchtime and suppertime on Monday, again at lunchtime and suppertime on Tuesday, and again at lunchtime on Wednesday.

In some embodiments in which the vibrating agitator is activated more than once, each activation of the vibrating agitator to be in the vibrating mode of operation is for a predetermined duration. For example, each activation of the vibrating agitator in the vibrating mode of operation may be for a duration of 1 hour, 1.5 hours, 2 hours, 2.5 hours, or 3 hours.

In such embodiments, power source 1108 has sufficient charge to enable activation of the vibrating agitator for the predetermined duration, multiple times. For example, power source 1108 may have sufficient charge to enable activation of the vibrating agitator in the vibrating mode of operation for at least 3 times, at least 4 times, or at least 5 times, where each activation is for the predetermined duration.

In some such embodiments, providing the vibration at a specific mealtime triggers a spontaneous bowel movement (SBM) or a complete spontaneous bowel movement (CSBM) of the subject at a later time of day than the predetermined time of day. For example, when the vibration is provided at lunchtime, i.e. between 12 pm and 3 pm or between 12 pm and 2 pm, the SBM or CSBM may occur around dinnertime, for example between 6 pm and 9 pm.

In some embodiments, use of capsule 1101, according to a treatment protocol, may increase the number of SBMs experienced by the subject, per week, by at least 1 or at least 2 SBMs.

In some embodiments, use of capsule 1101, according to a treatment protocol, may increase the number of CSBMs experienced by the subject, per week, by at least 1 or at least 2 CSBMs.

In some embodiments, the predetermined time of day is a user-specific time of day, or a user-specific mealtime at which the subject typically eats his/her meals. In some such embodiments, user input receiver 1128 of control unit 1120 (or an input receiver forming part of capsule 1101 (not shown)), is adapted to receive, from the user (e.g. the subject or a caregiver of the subject) information about the user-specific time of day or user-specific mealtime. Control unit 1120 may then provide the user-specific time of day or the user-specific mealtime to controller 1106, for example as part of an activation input or as a separate input, or may provide a suitable input or activation input based on the user-specific time of day or the user-specific mealtime. For example, if the subject is used to eating their meals at 11 am and 4 pm, these times may be provided as input to control unit 1120, which may then communicate these predetermined times of day to controller 1106 for activation of vibrating agitator 1104 at these times, or otherwise ensures that controller 1106 will activate the vibrating agitator at these times, for example by indicating a suitable delay time or providing an activation input at the user-specific time of day or the user-specific mealtime, as explained hereinabove. As another example, if the subject wishes for the capsule to vibrate at 4:00 am, the subject may provide this information as input to control unit 1120, which may then communicate this predetermined time to controller 1106 for activation of vibrating agitator 1104 at this time.

In some embodiments, the predetermined time of day is suited to the geographic region in which the capsule, the control unit 1120, or the subject, are located. For example, if the subject has ingested the capsule, and has changed time zones since ingesting the capsule (for example flew from New-York to Chicago), the predetermined time of day may be adjusted to correspond to the new time zone in which the user is located. In some embodiments, the predetermined time of day is a region-specific mealtime at which the people in a geographical region at which the subject (or capsule) is located typically eat their meals.

The times at which people typically have supper, or dinner, may vary greatly between different geographical regions. For example, in the U.S. typical supper times are between 5 pm and 8 pm or between 6 pm and 8 pm, whereas in Argentina most people only eat their supper between 9 pm and 11 pm, or even as late as midnight. Thus, the geographical region in which the capsule (and the subject) is located, may be used to identify the typical mealtimes of the subject.

In some such embodiments, location sensor 1114 of capsule 1101 may identify the location of the capsule, and provide the location or a region of the location to controller 1106. In other embodiments, location sensor 1124 of control unit 1120 may identify the location of the control unit, and may provide the location or a region of the location to controller 1106 as part of the activation input or as part of another input. Controller 1106 may then determine the predetermined time of day, or region-specific mealtime, at which the vibrating agitator 1104 should be in the operative mode of operation, based on the region. For example, controller 1106 may be preprogrammed with predetermined times of day for specific regions, and may select a suitable pre-programmed predetermined time of day based on the identified region. As another example, controller 1106 may access a database (not shown) to find the region-specific time of day, or region-specific mealtimes, for the identified region.

In yet other embodiments, location sensor 1124 of control unit 1120 may identify the location of the control unit, and may obtain, based on the identified location, the region-specific predetermined time(s) of day or region-specific mealtime(s), for example by accessing a pre-programmed list or by accessing a database (not shown). Control unit 1120 may then provide the obtained region-specific predetermined time(s) of day to controller 1106, substantially as described hereinabove. Alternatively, control unit 1120 may provide activation inputs, based on the obtained region-specific predetermined time(s) of day, to the controller 1106, without providing the region-specific predetermined time(s) of day to the controller, substantially as described hereinabove.

In some embodiments, in which the capsule 1101 is adapted to mitigate jetlag, the predetermined time of day is suited to the geographic region from which the capsule, the control unit 1120, or the subject, originate, or to the origin time zone of the subject. For example, if the subject has ingested the capsule, and has changed time zones since ingesting the capsule (for example flew from New-York to Chicago), the predetermined time of day is preferably maintained at the origin time zone of the origin location of the subject.

In some embodiments, the predetermined time of day is suited to the geographic region at which the capsule, the control unit 1120, or the subject, arrive, or to the destination time zone of the subject. For example, if the subject has ingested the capsule, and has changed time zones since ingesting the capsule (for example flew from New-York to Chicago), the predetermined time of day is preferably maintained at the destination time zone at which the subject has arrived.

In some embodiments, the capsule is adapted to operate in multiple cycles and/or during multiple predetermined times of day. In some such embodiments, in a first cycle or predetermined time of day, the time is suited to the geographic region from which the capsule, the control unit 1120, or the subject, originate, or to the origin time zone of the subject, and in a second cycle or predetermined time of day, the time is suited to the geographic region at which the capsule, the control unit 1120, or the subject, arrive, or to the destination time zone of the subject.

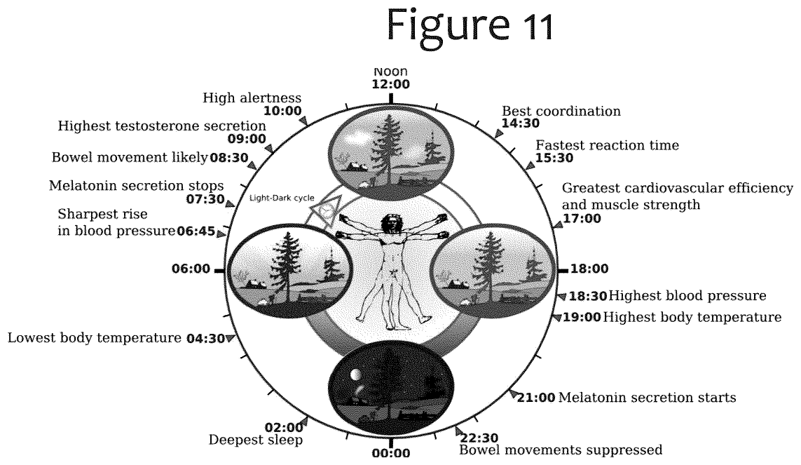

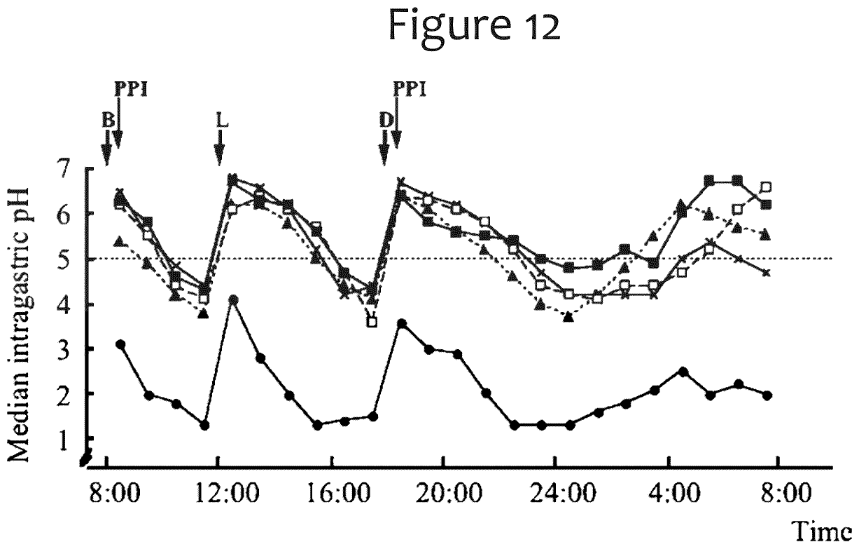

In some embodiments, the predetermined time of day is suited to the circadian cycle of the user, or to a default circadian cycle. For example, the predetermined time of day may be one occurring a predetermined duration prior to, or near, a time that, according to the circadian cycle, the user is likely to have a bowel movement, such that operation of the capsule 1101 may “assist” the gastrointestinal tract in generating, or completing, such a bowel movement. Additional examples relating to the circadian cycle are described hereinbelow with respect to FIG. 11. In some embodiments, the predetermined time of day may be selected to be a time of day at which the gastric pH of the user is relatively high, as explained in further detail hereinbelow with respect to FIG. 12.

In some embodiments, controller 1106 only activates vibrating agitator 1104 in the vibrating mode of operation at the predetermined time, if some minimum threshold duration has passed since capsule 1101 was activated to be in the operative state, since receipt of the activation input, or since the user ingested the capsule. For example, the capsule may be pre-programmed such that controller 1106 is adapted to activate vibrating agitator in the vibrating mode of operation from 6 am to 8 am, and from 6 pm to 8 pm, and the minimum threshold duration is four hours from ingestion of the capsule. If the subject ingests the capsule at 12 pm on Sunday, controller 1106 would cause vibrating agitator 1104 to operate in the vibrating mode of operation at 6 pm on Sunday, since the predetermined time of 6 pm is six hours following ingestion. However, if the subject ingests the capsule at 5 pm on Sunday, controller 1106 would cause vibrating agitator 1104 to operate in the vibrating mode of operation only at 6 am on Monday, since the first predetermined time of 6 pm is fewer than four hours following ingestion. Such a threshold duration may be particularly useful when it is desired that the capsule vibrate in a specific region or portion of the gastrointestinal tract, so that the delay time is required for the capsule to reach the specific region.

Relating to the characteristics of vibrating agitator 1104, the vibrating agitator may be any suitable mechanism that can be intermittently activated and can apply suitable forces onto capsule housing 1102.

In some embodiments, intermittently activated vibrating agitator 1104 may include a radial agitator adapted to exert radial forces on capsule housing 1102, in a radial direction with respect to the longitudinal axis of housing 1102. For example, the radial agitator may include an unbalanced weight attached to a shaft of an electric motor powered by a battery or by power source 1108, substantially as described in U.S. Pat. No. 9,707,150, which is incorporated by reference for all purposes as if fully set forth herein.

In some embodiments, intermittently activated vibrating agitator 1104 may include an axial agitator adapted to exert radial forces on the capsule housing 1102, in an axial direction with respect to a longitudinal axis of housing 1102. For example, the axial agitator may include an electric motor powered by a battery or by power source 1108, and an urging mechanism, associated with, and driven by, the electric motor, such that the urging mechanism adapted to exert said axial forces, substantially as described in U.S. Pat. No. 9,707,150. In some embodiments, the urging mechanism is adapted to exert the axial forces in opposite directions. In some embodiments, the urging mechanism is adapted to deliver at least a portion of the axial forces in a knocking mode.

In some embodiments, the forces exerted by intermittently activated vibrating agitator 1104 on capsule housing 1102 in the vibration mode of operation include radial forces in a radial direction with respect to the longitudinal axis of the housing and axial forces in an axial direction with respect to the longitudinal axis. In some embodiments, a single agitator exerts both the radial and the axial forces. In other embodiments, the axial forces are exerted by one agitator, and the radial forces are exerted by another, separate, agitator, where both agitators form part of intermittently activated vibrating agitator 1104.

In some embodiments, the intermittently activated vibrating agitator 1104 may include a magnet mounted onto a rotor adapted to exert a magnetic field as well as radial forces on capsule housing 1102. For example, such a magnetic vibrating agitator is described in U.S. patent application Ser. No. 15/058,216 filed on Mar. 2, 2016 (and published as US 2016/0310357), and entitled “PHYSIOTHERAPY DEVICE AND METHOD FOR CONTROLLING THE PHYSIOTHERAPY DEVICE”, which is incorporated by reference for all purposes as if fully set forth herein.

In some embodiments, housing 1102 may include first and second members, and vibrating agitator 1104 may include a mechanism adapted to effect a vibration by moving the first member of the housing in the opposite direction relative to the second member of the housing, substantially as described in U.S. Pat. No. 9,078,799, which is incorporated by reference for all purposes as if fully set forth herein.

In the vibrating mode of operation, intermittently activated vibrating agitator 1104 is adapted to have a plurality of vibration cycles, where each cycle includes a vibration duration followed by a repose duration. Forces are exerted by the vibrating agitator 1104 on capsule housing 1102 only during the vibration duration, and as such capsule housing 1102 only exerts forces on an environment thereof during the vibration duration.

In some embodiments, the number of vibration cycles per hour is in the range of to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, or 4 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, the total duration of one vibration cycle is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the cumulative duration of the vibrating mode of operation, or the cumulative duration during which vibration cycles are occurring, is in the range of 1 hour to 12 hours, 2 hours to 10 hours, 2 hours to 8 hours, 2 hours to 6 hours, 2 hours to 4 hours, or 2 hours to 3 hours. It will be appreciated that the cumulative duration of vibration cycles may be dependent on properties of power source 1108.

It will be appreciated by persons skilled in the art that the vibration mode of operation may be intermittent, or interrupted, such that vibrating agitator 1104 is operative in the vibration mode for a first duration, for example 30 minutes, then does not have any vibration cycles for a second duration, for example 1 hour, and then is operative in the vibration mode of operation and has vibration cycles for a third duration, for example two hours. The cumulative duration relates to the sum of all durations during which vibrating agitator 1104 was operative in the vibration mode of operation and included vibration cycles, including the vibration duration and the repose duration of each vibration cycle.

In some embodiments, vibrating agitator 1104 is configured to exert forces on the capsule housing 1102, such that a net force exerted by the capsule housing 1102 on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, vibrating agitator 1104 is configured to exert said forces on capsule housing 1102 to attain a capsule housing 1102 vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

It will be appreciated that the exact specifications of the capsule, such as the specific frequency and force ranges applicable to a specific capsule, are dependent on the specifications of the power source and of the vibrating agitator.

It will be further appreciated that a specific capsule may be controlled by the controller such that different vibrational frequencies may be attained and/or different net forces may be exerted, by the capsule in different vibration cycles of the capsule. Due to the natural distinction between subjects, use of multiple different parameters in different vibration cycles of a single capsule would allow the capsule to successfully treat multiple subjects, even if the personal optimal treatment for those subjects is not the same, as there is a higher chance that in at least some of the vibration cycles the activation parameters of the capsule would reach, or be close to, the optimal parameters for each specific subject.

Controller 1106 is adapted to control the operation of intermittently activated vibrating agitator 1104. Such control may include control of any one or more of the force applied by the vibrating agitator, the vibrational frequency reached, the times in which vibrating agitator 1104 operates in the vibration mode of operation, the vibration duration of each vibration cycle, the repose duration of each vibration cycle, the vibration cycle duration, and cumulative vibration duration of the vibrating agitators.

In some embodiments, controller 1106 is adapted to receive information relating to the desired vibration protocol from control unit 1120, prior to ingestion of the capsule or to activation thereof, or during the capsule's traversal of the subject's GI tract. For example, the information may be remotely transmitted from control unit 1120 to controller 1106, for example using a short-range wireless communication method. In some embodiments, the information is transmitted as a list of vibration parameters for effecting the vibration protocol. In some embodiments, the information is transmitted as executable code for effecting the first vibration protocol.

In some embodiments, the information includes one or more of at least one predetermined time of day, a time delay from receipt of the information to the at least one predetermined time of day, a region in which the control unit is located, a desired number of vibration cycles, a desired vibration duration in each vibration cycle, a desired repose duration in each vibration cycle, a desired cumulative vibration duration, and the like.

In some embodiments, controller 1106 is adapted to control vibrating agitator 1104 so that the capsule applies forces to an environment thereof to effect a mechanical stimulation of the wall of the gastrointestinal tract of the subject at the predetermined time(s) of day.

Turning to FIG. 9B, in one embodiment, the gastrointestinal treatment system includes only a vibrating ingestible capsule 1101b. Capsule 1101b includes a capsule housing or shell 1102, arranged along a longitudinal axis and having disposed therein a vibrating agitator 1104. Capsule 1101b further includes a controller 1106b, which may for example be, or include, a processor, is adapted to control operation of the vibrating agitator 1104, and at least one power source 1108 provides power to vibrating agitator 1104 and controller 1106b.

Shell 1102, vibrating agitator 1104, and power source 1108, are all substantially as described hereinabove with respect to FIG. 9A.

Capsule 1101b further includes a clock 1110b, and at least one sensor 1112, both functionally associated with controller 1106b and both powered by power source 1108.

The at least one sensor 1112 is adapted to sense at least one parameter within capsule 1101b or in an environment of capsule 1101b, to identify a specific condition in capsule 1101b or in the vicinity thereof, and to provide an activation input to controller 1106 in response to identification of the condition. Typically, the condition is indicative of the subject ingesting capsule 1101b. Examples of suitable sensors and conditions are provided hereinabove with respect to FIG. 9A.

It is a particular feature of the embodiment of FIG. 9B that controller 1106b is preprogrammed with at least one predetermined time of day as well as with a vibration protocol, as discussed hereinabove. Controller 1106b is adapted, in response to receiving from sensor(s) 1112 an indication that the capsule has been ingested, to activate clock 1110b to track the time of day, so as to identify occurrence of the at least one preprogrammed predetermined time of day, and to activate vibrating agitator 1104 to operate in the vibrating mode of operation, in accordance with the preprogrammed vibration protocol, at the at least one preprogrammed predetermined time of day.

In some embodiments, the capsule is in an inoperative state, until the receipt of the sensor indication, which causes controller 1106b to transition the capsule from the inoperative state to an operative state.

The at least one preprogrammed predetermined time of day is typically at least one default time of day, which may be, or may coincide with, at least one default mealtime, as described hereinabove with respect to FIG. 9A. As discussed hereinabove, a bowel movement supported by or triggered by the vibration may occur at a later time than the predetermined time of day.

As discussed hereinabove, the at least one time of day may include at least two predetermined times of day, such as two mealtimes. In some embodiments, the vibrating agitator may operate in the vibrating mode of operation at the at least one, or at least two, predetermined times of day, in two or more consecutive days.

In some embodiments, the at least one preprogrammed predetermined time of day is suited to a default circadian cycle, for example of a person who is awake during the day and sleeps during the night. For example, the predetermined time of day may be one occurring a predetermined duration prior to a time that, according to the default circadian cycle, a subject is likely to have a bowel movement, such that operation of the capsule 1101b may “assist” the gastrointestinal tract in generating, or completing, such a bowel movement. Additional examples relating to the circadian cycle are described hereinbelow with respect to FIG. 11.

In some embodiments, the clock 1110b may be set to a default time zone, for example a time zone at the location capsule 1101b is sold or provided. In such embodiments, the controller 1106b will be adapted to activate vibrating agitator 1104 to operate in the vibrating mode of operation at the preprogrammed predetermined time of day when that occurs in the default time zone, regardless of the actual location of the capsule. For example, if a subject received capsule 1101b from a physician in New York, the clock 1110b will be set to EST. If the subject then travels to London, and ingests the capsule in London, the capsule will still vibrate at the predetermined time of day according to EST, and not according to GMT.

In other embodiments, capsule 1101b further includes a location sensor 1114, such as a GPS or GLONASS receiver, functionally associated with controller 1106b, which is adapted to identify the geographic location of the capsule. In some such embodiments, when the user travels from an origin time zone to a destination time zone (such as from EST to GMT, as in the previous example), the location sensor 1114 provides to controller 1106b an indication of the change in time zone, and controller 1106b may reset clock 1110b to the destination time zone, or may otherwise compensate for the change in time zone, such that the vibration will occur at the preprogrammed predetermined time of day in accordance with the destination time zone.

As discussed hereinabove with respect to FIG. 9A, in some embodiments, controller 1106b only activates vibrating agitator 1104 in the vibrating mode of operation at the preprogrammed predetermined time(s), if some minimum threshold duration has passed since controller 1106b received an indication of ingesting of capsule 1101b.

Turning to FIG. 9C, in one embodiment, the gastrointestinal treatment system 1100c which includes a vibrating ingestible capsule 1101c, as well as a control unit 1120c. Capsule 1101c includes a capsule housing or shell 1102, arranged along a longitudinal axis and having disposed therein a vibrating agitator 1104. Capsule 1101c further includes a controller 1106c, which may for example be, or include, a processor, is adapted to control operation of the vibrating agitator 1104, and at least one power source 1108 provides power to vibrating agitator 1104 and controller 1106c.

Shell 1102, vibrating agitator 1104, and power source 1108, are all substantially as described hereinabove with respect to FIG. 9A.

Capsule 1101c further includes a clock 1110c and a remote input receiving mechanism 1116, such as a transceiver, both powered by power source 1108.

In the embodiment of FIG. 9C, controller 1106c is preprogrammed with at least one predetermined time of day as well as with a vibration protocol, as discussed hereinabove. However, capsule 1101c has no means for identifying when it has been ingested, for the controller 1106c to begin its operation.

Control unit 1120c, includes an input providing mechanism 1122, such as a transceiver, adapted to provide an activation input to transceiver 1116 of capsule 1101c, for example using the short-range wireless communication method indicated by arrow 1123. Typically, the activation input is provided from control unit 1120c to controller 1106c when the subject places the capsule 1101c on the control unit 1120c, operates a specific program or application on control unit 1120c, or otherwise indicates to control unit 1120c that he is about to ingest the capsule. The subject typically ingests the capsule immediately following provision of the activation input.

In some embodiments, control unit 1120c further includes a user input receiver (an input interface) 1128 via which the subject, or a caretaker thereof, may provide an indication that the subject is about to ingest the capsule 1101c.

Control unit 1120c, transceiver 1122, and user input receiver 1128, may be substantially as described hereinabove with respect to FIG. 9A.

In some embodiments, control unit 1120c is devoid of a timing mechanism.

Controller 1106c is adapted, in response to receipt of the activation input from control unit 1120c, to activate clock 1110c to track the time of day, so as to identify occurrence of the at least one preprogrammed predetermined time of day, and to activate vibrating agitator 1104 to operate in the vibrating mode of operation, in accordance with the preprogrammed vibration protocol, at the at least one preprogrammed predetermined time of day.

In some embodiments, the capsule is in an inoperative state, until the receipt of the activation input, which causes controller 1106c to transition the capsule from the inoperative state to an operative state.

The at least one preprogrammed predetermined time of day is typically at least one default time of day, which may be, or may coincide with, at least one default mealtime, as described hereinabove with respect to FIG. 9A. As discussed hereinabove, a bowel movement supported by or triggered by the vibration may occur at a later time than the predetermined time of day.

As discussed hereinabove, the at least one time of day may include at least two predetermined times of day, such as two mealtimes. In some embodiments, the vibrating agitator may operate in the vibrating mode of operation at the at least one, or at least two, predetermined times of day, in two or more consecutive days.

In some embodiments, the at least one preprogrammed predetermined time of day is suited to a default circadian cycle, for example of a person who is awake during the day and sleeps during the night. For example, the predetermined time of day may be one occurring a predetermined duration prior to a time that, according to the default circadian cycle, a subject is likely to have a bowel movement, such that operation of the capsule 1101c may “assist” the gastrointestinal tract in generating, or completing, such a bowel movement. Additional examples relating to the circadian cycle are described hereinbelow with respect to FIG. 11.

In some embodiments, the clock 1110c may be set to a default time zone, for example a time zone at the location capsule 1101c is sold or provided. In such embodiments, the controller 1106c will be adapted to activate vibrating agitator 1104 to operate in the vibrating mode of operation at the preprogrammed predetermined time of day when that occurs in the default time zone, regardless of the actual location of the capsule. For example, if a subject received capsule 1101c from a physician in New York, the clock 1110c will be set to EST. If the subject then travels to London, and ingests the capsule in London, the capsule will still vibrate at the predetermined time of day according to EST, and not according to GMT.

In other embodiments, capsule 1101c further includes a location sensor 1114, such as a GPS or GLONASS receiver, functionally associated with controller 1106c, which is adapted to identify the geographic location of the capsule. In some such embodiments, when the user travels from an origin time zone to a destination time zone (such as from EST to GMT, as in the previous example), the location sensor 1114 provides to controller 1106c an indication of the change in time zone, and controller 1106c may reset clock 1110c to the destination time zone, or may otherwise compensate for the change in time zone, such that the vibration will occur at the preprogrammed predetermined time of day in accordance with the destination time zone.

As discussed hereinabove with respect to FIG. 9A, in some embodiments, controller 1106c only activates vibrating agitator 1104 in the vibrating mode of operation at the preprogrammed predetermined time(s), if some minimum threshold duration has passed since controller 1106c received the activation input.

Turning now to FIG. 9D, the embodiment of FIG. 9D is substantially similar to that of FIG. 9C. The main difference between the embodiment of FIGS. 9C and 9D is that in the embodiment of FIG. 9D the gastrointestinal capsule is not preset with the predetermined time(s) of day, and receives these as part of the activation input, from the control unit.

As seen in FIG. 9D, the gastrointestinal treatment system 1100d which includes a vibrating ingestible capsule 1101d, as well as a control unit 1120d. Capsule 1101d includes a capsule housing or shell 1102, arranged along a longitudinal axis and having disposed therein a vibrating agitator 1104. Capsule 1101d further includes a controller 1106d, which may for example be, or include, a processor, is adapted to control operation of the vibrating agitator 1104, and at least one power source 1108 provides power to vibrating agitator 1104 and controller 1106d.

Shell 1102, vibrating agitator 1104, and power source 1108, are all substantially as described hereinabove with respect to FIG. 9A.

Capsule 1101d further includes a clock 1110d and a remote input receiving mechanism 1116, such as a transceiver, both powered by power source 1108.

Control unit 1120d, includes an input providing mechanism 1122, such as a transceiver, adapted to provide an activation input to transceiver 1116 of capsule 1101d, for example using the short-range wireless communication method indicated by arrow 1123. The activation input includes at least one predetermined time of day at which the capsule 1101d should be operative in the vibrating mode of operation. In some embodiments, the activation input may further include a vibration protocol, substantially as discussed hereinabove with respect to FIG. 9A.

Typically, the activation input is provided from control unit 1120d to controller 1106d when the subject places the capsule 1101d on the control unit 1120d, operates a specific program or application on control unit 1120d, or otherwise indicates to control unit 1120d that he is about to ingest the capsule. The subject typically ingests the capsule immediately following provision of the activation input.

In some embodiments, control unit 1120d further includes a user input receiver (an input interface) 1128 via which the subject, or a caretaker thereof, may provide an indication that the subject is about to ingest the capsule 1101d.

Control unit 1120d, may further include a timing mechanism 1126 which is typically a clock, and may, in some embodiments, include a location sensor 1124, as explained in further detail herein. Control unit 1120d, transceiver 1122, clock 1126, location sensor 1124, and user input receiver 1128, may be substantially as described hereinabove with respect to FIG. 9A.

In some embodiments, the at least one predetermined time of day may be preset or preprogrammed in the control unit 1120d. In some embodiments, the at least one time of day may be a default time of day, which may be, or may coincide with, at least one default mealtime, as described hereinabove with respect to FIG. 9A.

As discussed hereinabove, the at least one time of day may include at least two predetermined times of day, such as two mealtimes. In some embodiments, the vibrating agitator may operate in the vibrating mode of operation at the at least one, or at least two, predetermined times of day, in two or more consecutive days.

In other embodiments, the control unit 1120d may be adapted to compute or otherwise determine the at least one predetermined time of day.

In some such embodiments, the control unit 1120d may determine the predetermined time(s) of day based on a geographical location of the control unit, for example as identified by the location sensor 1124, as described hereinabove with respect to FIG. 9A.

In other such embodiments, the control unit 1120d may determine the predetermined time(s) of day based on input received via user input receiver via user input receiver 1128, and/or based on input received from a sensor, for example the toilet bowl sensor described hereinabove, or a sleep sensor worn by the user (e.g. Fitbit® activity tracker). In some embodiments, the control unit 1120d determines the time(s) of day is suited to the user's circadian cycle, for example based on a sleep schedule, a defecation schedule, a vitality schedule, and/or a meal schedule provided as part of the input. For example, the predetermined time of day may be one occurring a predetermined duration prior to a time that, according to the default circadian cycle or to the subject's circadian cycle, as established by the received input, the subject is likely to have a bowel movement, such that operation of the capsule 1101d may “assist” the gastrointestinal tract in generating, or completing, such a bowel movement. Additional examples relating to the circadian cycle are described hereinbelow with respect to FIG. 11.

Controller 1106d is adapted, in response to receipt of the activation input from control unit 1120d, to activate clock 1110d to track the time of day, so as to identify occurrence of the at least one predetermined time of day received as part of the activation input, and to activate vibrating agitator 1104 to operate in the vibrating mode of operation, in accordance with a preprogrammed vibration protocol or a vibration protocol received as part of the activation input, at the at least one predetermined time of day.

In some embodiments, the capsule is in an inoperative state, until the receipt of the activation input, which causes controller 1106d to transition the capsule from the inoperative state to an operative state.

In some embodiments, the clock 1110d may be set to a default time zone, for example a time zone at the location capsule 1101d is sold or provided. In some such embodiments, the controller 1106d will be adapted to activate vibrating agitator 1104 to operate in the vibrating mode of operation at the predetermined time of day when that occurs in the default time zone, regardless of the actual location of the capsule. For example, if a subject received capsule 1101d from a physician in New York, the clock 1110d will be set to EST. If the subject then travels to London, and ingests the capsule in London, the capsule will still vibrate at the predetermined time of day according to EST, and not according to GMT.

In other embodiments, when providing the activation input, control unit 1120d may use clock 1126 and location sensor 1124 thereof to compensate for the change in time zone, such that the time of day provided to controller 1106d is corrected to match the time zone at which the control unit is currently located.

In yet other embodiments, capsule 1101d further includes a location sensor 1114, such as a GPS or GLONASS receiver, functionally associated with controller 1106d, which is adapted to identify the geographic location of the capsule. In some such embodiments, when the user travels from an origin time zone to a destination time zone (such as from EST to GMT, as in the previous example), the location sensor 1114 provides to controller 1106d an indication of the change in time zone, and controller 1106d may reset clock 1110d to the destination time zone, or may otherwise compensate for the change in time zone, such that the vibration will occur at the predetermined time of day in accordance with the destination time zone.

As discussed hereinabove with respect to FIG. 9A, in some embodiments, controller 1106d only activates vibrating agitator 1104 in the vibrating mode of operation at the predetermined time(s), if some minimum threshold duration has passed since controller 1106d received the activation input (which is indicative of the subject planning to ingest the capsule).

Turning now to FIG. 9E, the embodiment of FIG. 9E is substantially similar to that of FIG. 9D. The main difference between the embodiment of FIGS. 9D and 9E is that in the embodiment of FIG. 9E the activation input does not include the predetermined time of day, but rather includes a time delay, from the current time, to the predetermined time of day.

As seen in FIG. 9E, the gastrointestinal treatment system 1100e which includes a vibrating ingestible capsule 1101e, as well as a control unit 1120e. Capsule 1101e includes a capsule housing or shell 1102, arranged along a longitudinal axis and having disposed therein a vibrating agitator 1104. Capsule 1101e further includes a controller 1106e, which may for example be, or include, a processor, is adapted to control operation of the vibrating agitator 1104, and at least one power source 1108 provides power to vibrating agitator 1104 and controller 1106e.

Shell 1102, vibrating agitator 1104, and power source 1108, are all substantially as described hereinabove with respect to FIG. 9A.

Capsule 1101e further includes a timer 1110e and a remote input receiving mechanism 1116, such as a transceiver, both powered by power source 1108.

Control unit 1120e, includes an input providing mechanism 1122, such as a transceiver, adapted to provide an activation input to transceiver 1116 of capsule 1101e, for example using the short-range wireless communication method indicated by arrow 1123. The activation input includes at least one delay time, from a current time of day to at least one predetermined time of day at which the capsule 1101e should be operative in the vibrating mode of operation. In some embodiments, the activation input may further include a vibration protocol, substantially as discussed hereinabove with respect to FIG. 9A.

Typically, the activation input is provided from control unit 1120e to controller 1106e when the subject places the capsule 1101e on the control unit 1120e, operates a specific program or application on control unit 1120e, or otherwise indicates to control unit 1120e that he is about to ingest the capsule. The subject typically ingests the capsule immediately following provision of the activation input.

In some embodiments, control unit 1120e further includes a user input receiver (an input interface) 1128 via which the subject, or a caretaker thereof, may provide an indication that the subject is about to ingest the capsule 1101e.

Control unit 1120e, further includes a timing mechanism 1126 which is typically a clock, and may, in some embodiments, include a location sensor 1124, as explained in further detail herein. Control unit 1120e, transceiver 1122, clock 1126, location sensor 1124, and user input receiver 1128, may be substantially as described hereinabove with respect to FIG. 9A.

In some embodiments, the at least one predetermined time of day may be preset or preprogrammed in the control unit 1120e. In some embodiments, the at least one time of day may be a default time of day, which may be, or may coincide with, at least one default mealtime, as described hereinabove with respect to FIG. 9A.

As discussed hereinabove, the at least one time of day may include at least two predetermined times of day, such as two mealtimes. In some embodiments, the vibrating agitator may operate in the vibrating mode of operation at the at least one, or at least two, predetermined times of day, in two or more consecutive days.

In other embodiments, the control unit 1120e may be adapted to compute or otherwise determine the at least one predetermined time of day.

In some such embodiments, the control unit 1120e may determine the predetermined time(s) of day based on a geographical location of the control unit, for example as identified by the location sensor 1124, as described hereinabove with respect to FIG. 9A.

In other such embodiments, the control unit 1120e may determine the predetermined time(s) of day based on input received via user input receiver via user input receiver 1128, and/or based on input received from a sensor, for example the toilet bowl sensor described hereinabove, or a sleep sensor worn by the user (e.g. Fitbit® activity tracker). In some embodiments, the control unit 1120e determines the time(s) of day is suited to the user's circadian cycle, for example based on a sleep schedule, a defecation schedule, a vitality schedule, and/or a meal schedule provided as part of the input. For example, the predetermined time of day may be one occurring a predetermined duration prior to a time that, according to the default circadian cycle or to the subject's circadian cycle, as established by the received input, the subject is likely to have a bowel movement, such that operation of the capsule 1101e may “assist” the gastrointestinal tract in generating, or completing, such a bowel movement. Additional examples relating to the circadian cycle are described hereinbelow with respect to FIG. 11.

Controller 1106e is adapted, in response to receipt of the activation input from control unit 1120e, to activate timer 1110e to track passage of time until completion of the delay time, and to activate vibrating agitator 1104 to operate in the vibrating mode of operation, in accordance with a preprogrammed vibration protocol or a vibration protocol received as part of the activation input, at a time coinciding with the at least one predetermined time of day.

In some embodiments, the capsule is in an inoperative state, until the receipt of the activation input, which causes controller 1106e to transition the capsule from the inoperative state to an operative state.

In other embodiments, when providing the activation input, control unit 1120e may use clock 1126 and location sensor 1124 thereof to compensate for a change in time zone, such as would be caused by a user travelling from New York to London, such that the delay time provided to controller 1106e is corrected so that the vibration will occur at the predetermined time of day in the destination location, or at which the subject is currently located.

As discussed hereinabove with respect to FIG. 9A, in some embodiments, controller 1106e only activates vibrating agitator 1104 in the vibrating mode of operation following the delay time, if some minimum threshold duration has passed since controller 1106e received the activation input (which is indicative of the subject planning to ingest the capsule).

It is a particular feature of the embodiment of FIG. 9E that, although the capsule 1101e is not (necessarily) aware of the time of day, the control unit controls the capsule such that the vibrating mode of operation occurs at the predetermined time(s) of day.

Turning now to FIG. 9F, the embodiment of FIG. 9F is distinct from the embodiments of FIGS. 9B to 9E in that the activation input is provided at the predetermined time of day, after the user has ingested the gastrointestinal capsule.

As seen in FIG. 9F, the gastrointestinal treatment system 1100f which includes a vibrating ingestible capsule 1101f, as well as a control unit 1120f. Capsule 1101f includes a capsule housing or shell 1102, arranged along a longitudinal axis and having disposed therein a vibrating agitator 1104. Capsule 1101f further includes a controller 1106f, which may for example be, or include, a processor, is adapted to control operation of the vibrating agitator 1104, and at least one power source 1108 provides power to vibrating agitator 1104 and controller 1106f.

Shell 1102, vibrating agitator 1104, and power source 1108, are all substantially as described hereinabove with respect to FIG. 9A.

Capsule 1101f further includes a remote input receiving mechanism 1116, such as a transceiver, powered by power source 1108. Transceiver 1116 is sufficiently strong, or uses a suitable protocol, such that transceiver 1116 can receive communications from outside the body, while capsule 1101f traverses the GI tract.

Capsule 1101f is devoid of a timing mechanism.

Control unit 1120f, includes an input providing mechanism 1122, such as a transceiver, adapted to communicate and to provide an activation input to transceiver 1116 of capsule 1101f, even following ingestion of the capsule, for example using the short-range wireless communication method indicated by arrow 1123. The activation input indicates to the controller 1106f that the vibrating agitator should be operated in the first vibrating mode of operation now, at the time of receipt of the activation input. In some embodiments the activation input may include the vibration protocol to be used, substantially as described hereinabove with respect to FIG. 9A, and/or the duration for which the capsule should vibrate, in response to the activation input.

In some embodiments, a link between the capsule 1101f and the control unit 1120f may be formed when the subject places the capsule 1101f on the control unit 1120f, prior to ingestion of the capsule.

Control unit 1120f, further includes a timing mechanism 1126 which is typically a clock, and may, in some embodiments, include a location sensor 1124, as explained in further detail herein. Control unit 1120f, transceiver 1122, clock 1126, location sensor 1124, and user input receiver 1128, may be substantially as described hereinabove with respect to FIG. 9A.

In some embodiments, the at least one predetermined time of day may be preset or preprogrammed in the control unit 1120f. In some embodiments, the at least one time of day may be a default time of day, which may be, or may coincide with, at least one default mealtime, as described hereinabove with respect to FIG. 9A.

As discussed hereinabove, the at least one time of day may include at least two predetermined times of day, such as two mealtimes. In some embodiments, the vibrating agitator may be activated, e.g. by the control unit, operate in the vibrating mode of operation at the at least one, or at least two, predetermined times of day, in two or more consecutive days.

In other embodiments, the control unit 1120f may be adapted to compute or otherwise determine the at least one predetermined time of day.

In some such embodiments, the control unit 1120f may determine the predetermined time(s) of day based on a geographical location of the control unit, for example as identified by the location sensor 1124, as described hereinabove with respect to FIG. 9A.

In other such embodiments, the control unit 1120f may determine the predetermined time(s) of day based on input received via user input receiver via user input receiver 1128, and/or based on input received from a sensor, for example the toilet bowl sensor described hereinabove, or a sleep sensor worn by the user (e.g. Fitbit® activity tracker). In some embodiments, the control unit 1120f determines the time(s) of day is suited to the user's circadian cycle, for example based on a sleep schedule, a defecation schedule, a vitality schedule, and/or a meal schedule provided as part of the input. For example, the predetermined time of day may be one occurring a predetermined duration prior to a time that, according to the default circadian cycle or to the subject's circadian cycle, as established by the received input, the subject is likely to have a bowel movement, such that operation of the capsule 1101f may “assist” the gastrointestinal tract in generating, or completing, such a bowel movement. Additional examples relating to the circadian cycle are described hereinbelow with respect to FIG. 11.

Following ingestion of the capsule, when control unit 1120f determines that the predetermined time of day has arrived, for example by tracking time using clock 1126, it provides an activation input to controller 1106f. Controller 1106f is adapted, in response to receipt of the activation input from control unit 1120f, to activate vibrating agitator 1104 to operate in the vibrating mode of operation, in accordance with a preprogrammed vibration protocol or a vibration protocol received as part of the activation input, at a time coinciding with the at least one predetermined time of day.

In some embodiments, the capsule is in an inoperative state, until the linking of the capsule and the control unit, which causes controller 1106f to transition the capsule from the inoperative state to an operative state.

It is a particular feature of the embodiment of FIG. 9F that, although the capsule 1101f is not aware of the time of day, the control unit controls the capsule such that the vibrating mode of operation occurs at the predetermined time(s) of day.

Reference is now additionally made to FIGS. 10A to 10F, which are schematic flowcharts of embodiments of methods for treating the gastrointestinal tract according to the present invention, the treatment being based on use of the gastrointestinal treatment system of FIGS. 9A to 9F, respectively. For example, the methods of treatment illustrated in FIGS. 10A to 10F may be used for treating an ailment of the gastrointestinal tract, or for mitigating jetlag of the user.

It will be appreciated by people of skill in the art that the methods described herein may be used for treatment of various ailments of the gastrointestinal tract, including constipation, a sensation of straining while defecating, a sensation of gastric bloating, diarrhea, and gastroparesis.

It will further be appreciated by people of skill in the art that the methods described herein may be used for mitigating at least an acute constipation symptom of jetlag of a subject.

Turning to FIG. 10A, it is seen that at step 1200, initially the treatment protocol for the subject is determined, for example by a treating physician or medical practitioner. The treatment protocol may indicate the number of treatment sessions per week or per other time duration, the time of day at which a capsule should be ingested, one or more predetermined times of day at which the capsule should be operative, and/or may indicate the vibration protocol of the capsule.

In some embodiments, for example when the treatment is intended to mitigate jetlag of a travelling subject, the treatment protocol may be selected at least partially according to a travel plan of the subject. As such, the treatment protocol may take into consideration, for example, times the subject will be in travelling (e.g. on an airplane, boat, or train), the origin time zone (from which the subject will be leaving), the destination time zone (at which the subject will be arriving), and the time of day at which the subject will be arriving at the destination.

At step 1202, a controller 1106 of an ingestible capsule 1101 is provided with a predetermined time, or times, of day at which the vibrating agitator should be operated in the vibrating mode of operation. In some embodiments the controller is also provided with the number of consecutive days that the vibrating agitator should be operated in the vibrating mode of operation, at the predetermined time(s). In some embodiments, the controller may also be provided with a specification of a time zone to which the predetermined time(s) of day should relate, for example an origin time zone or a destination time zone of a travelling subject.

In some embodiments, at step 1204 controller 1106 may optionally receive, or be programmed with, a desired vibration protocol for the subject.

At step 1206, the capsule may be activated for use, by transitioning the capsule from an inoperative state to an operative state, for example by receipt of an activation input.

The subject ingests the capsule at step 1208, and at step 1210 controller 1106 control vibrating agitator 1104 such that the vibrating mode of operation occurs at the predetermined time(s) of day, for the defined number of consecutive days.

In some embodiments, providing of the predetermined time(s) at step 1202 and/or providing the desired vibration protocol for the subject at step 1204 occurs at the time of manufacturing of the capsule, for example by pre-programming the time(s) into the controller.

In some embodiments, providing of the predetermined time(s) at step 1202 and/or providing the desired vibration protocol for the subject at step 1204 may be effected by a control unit, such as control unit 1120 of FIG. 9A.

For example, control unit 1120 may provide to controller 1106 the predetermined time of day, a user-specific predetermined time of day, a region-specific predetermined time of day, or a time-zone specific time of day as described hereinabove. In such embodiments, step 1202 may be carried out at any time prior to operating to the vibrating agitator in the vibrating mode of operation at step 1210, and specifically may be carried out prior to ingestion of the capsule by the subject, or following the subject ingesting the capsule.

For example, the programming of the vibration protocol and/or of the predetermined time of day may include remotely transmitting the desired vibration protocol and/or predetermined time of day from the control unit 1120 to the controller 1106, for example using a short-range wireless communication method. In some embodiments, the desired vibration protocol is transmitted as a list of vibration parameters for effecting the vibration protocol. In some embodiments, the desired vibration protocol is transmitted as executable code for effecting the vibration protocol.

In some embodiments, the control unit provides the predetermined time of day to controller 1106 at the predetermined time of day, which is equivalent to giving the command “operate the vibrating agitator 1104 now”. In such embodiments, step 1202 is carried out following the subject ingesting the capsule at step 1208.

As mentioned above, the capsule is activated for use at step 1206. Typically, the capsule is activated by receipt of an activation input.

As discussed hereinabove, in some embodiments the activation input may be received from the control unit 1120 or from sensors within the capsule sensing that the capsule has been ingested or that a user has carried out an activation motion with the capsule.

In some embodiments, the capsule is activated prior to the user ingesting the capsule at step 1208, for example by a signal from the control unit or by the user carrying out an activation motion. In other embodiments, the activation input is provided at the time of ingestion or immediately thereafter, for example by sensors sensing a change in the environment of the capsule due to its ingestion, as described at length hereinabove. In yet other embodiments, the activation input may be provided remotely when the capsule is already in the body of the subject, for example by remote communication from control module 1120.

Following activation of capsule 1101, or together therewith, capsule 1101 is ingested by the subject, and begins to travel through the gastrointestinal tract of the subject, as seen at step 1208.

Operation of vibrating agitator 1104 in the vibrating mode of operation at step 1210 effects vibration of capsule housing 1102, as described hereinabove, such that the housing exerts vibrations on the environment surrounding the capsule. Specifically, vibration of capsule housing 1102 may be intended to effect a mechanical stimulation of the wall of the gastrointestinal tract at the predetermined time of day.

In some embodiments, vibration of the capsule at the predetermined time of day, which effects the mechanical stimulation, triggers the subject to have an SBM or a CSBM at a later time of day than the vibration. For example, vibration of the capsule between 12 pm and 2 pm may trigger the subject to have a CSBM between 6 pm and 8 pm. In some embodiments, for example when the method of FIG. 10A is used to treat an ailment of the gastrointestinal tract, a treatment session as defined in steps 1202 to 1210 may be repeatedly administered to the subject as specified in the treatment protocol for the subject, determined or obtained at step 1200. In some embodiments, the treatment protocol includes administering a plurality of treatment sessions to the subject. In some embodiments, the treatment protocol includes administering at least one treatment session to the subject per week, over a treatment period of at least two weeks, at least at least three weeks, at least four weeks, at least five weeks, at least six weeks, or at least eight weeks. In some embodiments, the treatment protocol includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

Turning now to FIG. 10B, it is seen that at step 1200b, initially the treatment procedure for the subject may be determined, for example by a treating physician or medical practitioner. The treatment procedure may indicate the number of treatment sessions per week or per other time duration and/or the time of day at which a capsule should be ingested.

At step 1202b, the subject obtains a vibrating ingestible capsule, for example capsule 1101b as described hereinabove with respect to FIG. 9B. A controller 1106b of the capsule is preprogrammed with a predetermined time, or times, of day at which the vibrating agitator should be operated in the vibrating mode of operation, with the number of consecutive days that the capsule should be operated in the vibrating mode of operation at the predetermined time(s), and with a vibration protocol for the vibrating mode of operation.

The subject ingests the capsule at step 1208. Upon ingestion of the capsule, sensor 1112 of capsule 1101b provides to controller 1106b an input indicating that the capsule has been ingested, and the controller track time and activates vibrating agitator 1104 such that the vibrating mode of operation occurs at the predetermined time(s) of day. Operation of vibrating agitator 1104 in the vibrating mode of operation effects vibration of capsule housing 1102, as described hereinabove, such that the housing exerts vibrations on the environment surrounding the capsule. Specifically, vibration of capsule housing 1102 may be intended to effect a mechanical stimulation of the wall of the gastrointestinal tract at the predetermined time of day.

In some embodiments, vibration of the capsule at the predetermined time of day, which effects the mechanical stimulation, triggers the subject to have an SBM or a CSBM at a later time of day than the vibration. For example, vibration of the capsule between 12 pm and 2 pm may trigger the subject to have a CSBM between 6 pm and 8 pm.

In some embodiments, for example when the method of FIG. 10B is used to treat an ailment of the gastrointestinal tract, a treatment session as defined in steps 1202b and 1208 may be repeatedly administered to the subject as specified in the treatment procedure for the subject, determined or obtained at step 1200b. In some embodiments, the treatment procedure includes administering a plurality of treatment sessions to the subject, substantially as described hereinabove with respect to FIG. 10B.

Turning now to FIG. 10C, it is seen that at step 1200c, initially the treatment procedure for the subject may be determined, for example by a treating physician or medical practitioner. The treatment procedure may indicate the number of treatment sessions per week or per other time duration and/or the time of day at which a capsule should be ingested.

At step 1202c, the subject obtains a vibrating ingestible capsule, for example capsule 1101c as described hereinabove with respect to FIG. 9C. A controller 1106c of the capsule is preprogrammed with a predetermined time, or times, of day at which the vibrating agitator should be operated in the vibrating mode of operation, with the number of consecutive days that the capsule should be operated in the vibrating mode of operation at the predetermined time(s), and with a vibration protocol for the vibrating mode of operation.

At step 1206c, the subject causes a control unit, distinct from the capsule, such as control unit 1120c of FIG. 9C, to provide an activation input to the capsule 1101c. For example, the subject (or a caregiver thereof) may place the capsule in or on the control unit for a predetermined duration, so as to provide the activation input from the control unit to the capsule. As another example, the subject may operate an application running on the control unit, or otherwise provide a user input to the control unit, so as to trigger the control unit to send an activation input to the capsule.

Following provision of the activation input at step 1206c, the subject ingests the capsule at step 1208. Upon receipt of the activation input from control unit 1120c (and regardless of actual ingestion of the capsule), controller 1106c tracks time and activates vibrating agitator 1104 such that the vibrating mode of operation occurs at the predetermined time(s) of day. Operation of vibrating agitator 1104 in the vibrating mode of operation effects vibration of capsule housing 1102, as described hereinabove, such that the housing exerts vibrations on the environment surrounding the capsule. Specifically, vibration of capsule housing 1102 may be intended to effect a mechanical stimulation of the wall of the gastrointestinal tract at the predetermined time of day.

In some embodiments, vibration of the capsule at the predetermined time of day, which effects the mechanical stimulation, triggers the subject to have an SBM or a CSBM at a later time of day than the vibration. For example, vibration of the capsule between 12 pm and 2 pm may trigger the subject to have a CSBM between 6 pm and 8 pm.

In some embodiments, for example when the method of FIG. 10C is used to treat an ailment of the gastrointestinal tract, a treatment session as defined in steps 1202c to 1208 may be repeatedly administered to the subject as specified in the treatment procedure for the subject, determined or obtained at step 1200c. In some embodiments, the treatment procedure includes administering a plurality of treatment sessions to the subject, substantially as described hereinabove with respect to FIG. 10C.

Turning now to FIG. 10D, it is seen that at step 1200d, initially the treatment protocol for the subject is determined, for example by a treating physician or medical practitioner. The treatment protocol may indicate the number of treatment sessions per week or per other time duration, the time of day at which a capsule should be ingested, one or more predetermined times of day at which the capsule should be operative, a number of consecutive days that the capsule should be operated in the vibrating mode of operation at the predetermined time(s), and/or may indicate the vibration protocol of the capsule. Typically, at least a portion of the treatment protocol is provided to the control unit, such as control unit 1120d of FIG. 9D.

At step 1202d, the subject obtains, or is provided, a vibrating ingestible capsule, for example capsule 1101d as described hereinabove with respect to FIG. 9D. In some embodiments, the capsule may be provided by a medical practitioner, such as a treating physician.

At step 1206d, the subject causes the control unit, to provide an activation input to the capsule 1101d. The activation input includes the at least one time of day at which the capsule should operate in the vibrating mode of operation, and in some embodiments may also include the number of consecutive days that the capsule should be operated in the vibrating mode of operation at the predetermined time(s), and/or the vibration protocol as determined at step 1200d. For example, the subject (or a caregiver thereof) may place the capsule in or on the control unit for a predetermined duration, so as to provide the activation input from the control unit to the capsule. As another example, the subject may operate an application running on the control unit, or otherwise provide a user input to the control unit, so as to trigger the control unit to send an activation input to the capsule.

Following provision of the activation input at step 1206d, the subject ingests the capsule at step 1208. Upon receipt of the activation input from control unit 1120d (and regardless of actual ingestion of the capsule), controller 1106c tracks time and activates vibrating agitator 1104 such that the vibrating mode of operation occurs at the predetermined time(s) of day received with the activation input. Operation of vibrating agitator 1104 in the vibrating mode of operation effects vibration of capsule housing 1102, as described hereinabove, such that the housing exerts vibrations on the environment surrounding the capsule. Specifically, vibration of capsule housing 1102 may be intended to effect a mechanical stimulation of the wall of the gastrointestinal tract at the predetermined time of day.

In some embodiments, vibration of the capsule at the predetermined time of day, which effects the mechanical stimulation, triggers the subject to have an SBM or a CSBM at a later time of day than the vibration. For example, vibration of the capsule between 12 pm and 2 pm may trigger the subject to have a CSBM between 6 pm and 8 pm.

In some embodiments, at optional step 1203d, a user input may be received at the control unit, for example via a user interface thereof. For example, the user input may include information pertaining to the circadian cycle of the subject, such as the subject's sleep schedule and/or meal schedule. The user input may be provided by the subject, medical personnel, or by a caregiver of the subject. The user input may be used to set, or to adjust, the predetermined time(s) of day for the subject.

In some embodiments, the predetermined time(s) of day is preset in the control unit, or is determined at step 1200d and is provided to the control unit.

In other embodiments, at optional step 1204d, the control unit computes the predetermined time(s) of day, prior to providing the activation input to the capsule. The computation may be based on user input received at step 1203d or on a location of the control unit as identified by location sensor 1124.

In some embodiments, for example when the method of FIG. 10D is used to treat an ailment of the gastrointestinal tract, a treatment session as defined in steps 1202d to 1208 may be repeatedly administered to the subject as specified in the treatment procedure for the subject, determined or obtained at step 1200d. In some embodiments, the treatment procedure includes administering a plurality of treatment sessions to the subject, substantially as described hereinabove with respect to FIG. 10D.

Turning now to FIG. 10E, it is seen that at step 1200e, initially the treatment protocol for the subject is determined, for example by a treating physician or medical practitioner. The treatment protocol may indicate the number of treatment sessions per week or per other time duration, the time of day at which a capsule should be ingested, one or more predetermined times of day at which the capsule should be operative, a number of consecutive days that the capsule should be operated in the vibrating mode of operation at the predetermined time(s), and/or may indicate the vibration protocol of the capsule. Typically, at least a portion of the treatment protocol is provided to the control unit, such as control unit 1120e of FIG. 9E.

At step 1202e, the subject obtains, or is provided, a vibrating ingestible capsule, for example capsule 1101e as described hereinabove with respect to FIG. 9E. In some embodiments, the capsule may be provided by a medical practitioner, such as a treating physician.

At step 1206e, the subject causes the control unit, to provide an activation input to the capsule 1101e. The activation input includes at least one-time delay from a current time to at least one time of day at which the capsule should operate in the vibrating mode of operation, and in some embodiments may also include the number of consecutive days that the capsule should be operated in the vibrating mode of operation at the predetermined time(s), and/or the vibration protocol as determined at step 1200e. The at least one-time delay may be computed by the control unit, based on the current time and the predetermined time(s) of day. For example, the subject (or a caregiver thereof) may place the capsule in or on the control unit for a predetermined duration, so as to provide the activation input from the control unit to the capsule. As another example, the subject may operate an application running on the control unit, or otherwise provide a user input to the control unit, so as to trigger the control unit to send an activation input to the capsule.

Following provision of the activation input at step 1206e, the subject ingests the capsule at step 1208. Upon receipt of the activation input from control unit 1120e (and regardless of actual ingestion of the capsule), controller 1106e tracks passage of time until the at least one time delay has been completed, and activates vibrating agitator 1104 to operate in the vibrating mode of operation, such that the vibrating mode of operation coincides with the predetermined time(s) of day. Operation of vibrating agitator 1104 in the vibrating mode of operation effects vibration of capsule housing 1102, as described hereinabove, such that the housing exerts vibrations on the environment surrounding the capsule. Specifically, vibration of capsule housing 1102 may be intended to effect a mechanical stimulation of the wall of the gastrointestinal tract at the predetermined time of day.

In some embodiments, vibration of the capsule at the predetermined time of day, which effects the mechanical stimulation, triggers the subject to have an SBM or a CSBM at a later time of day than the vibration. For example, vibration of the capsule between 12 pm and 2 pm may trigger the subject to have a CSBM between 6 pm and 8 pm.

In some embodiments, at optional step 1203e, a user input may be received at the control unit, for example via a user interface thereof. For example, the user input may include information pertaining to the circadian cycle of the subject, such as the subject's sleep schedule and/or meal schedule. The user input may be provided by the subject, medical personnel, or by a caregiver of the subject. The user input may be used to set, or to adjust, the predetermined time(s) of day for the subject.

In some embodiments, the predetermined time(s) of day is preset in the control unit, or is determined at step 1200e and is provided to the control unit.

In other embodiments, at optional step 1204e, the control unit computes the predetermined time(s) of day, as well as the delay time(s), prior to providing the activation input to the capsule. The computation of the predetermined time(s) of day may be based on user input received at step 1203e or on a location of the control unit as identified by location sensor 1124.

In some embodiments, for example when the method of FIG. 10E is used to treat an ailment of the gastrointestinal tract, a treatment session as defined in steps 1202e to 1208 may be repeatedly administered to the subject as specified in the treatment procedure for the subject, determined or obtained at step 1200e. In some embodiments, the treatment procedure includes administering a plurality of treatment sessions to the subject, substantially as described hereinabove with respect to FIG. 10E.

Turning now to FIG. 10F, it is seen that at step 1200f, initially the treatment protocol for the subject may be determined, for example by a treating physician or medical practitioner. The treatment protocol may indicate the number of treatment sessions per week or per other time duration, the time of day at which a capsule should be ingested, one or more predetermined times of day at which the capsule should be operative, a number of consecutive days that the capsule should be operated in the vibrating mode of operation at the predetermined time(s), and/or may indicate the vibration protocol of the capsule. Typically, at least a portion of the treatment protocol is provided to the control unit, such as control unit 1120f of FIG. 9F.

At step 1202f, the subject obtains, or is provided, a vibrating ingestible capsule, for example capsule 1101f as described hereinabove with respect to FIG. 9F. In some embodiments, the capsule may be provided by a medical practitioner, such as a treating physician.

The subject ingests the capsule at step 1208.

Subsequently, at step 1210f, at the at least one predetermined time of day, the control unit 1120f provides an activation input to the capsule 1101f. In some embodiments, the activation input may include the vibration protocol as determined at step 1200f. Upon receipt of the activation input from control unit 1120f, controller 1106f, substantially immediately, activates vibrating agitator 1104 to operate in the vibrating mode of operation, such that the vibrating mode of operation coincides with the predetermined time(s) of day. Operation of vibrating agitator 1104 in the vibrating mode of operation effects vibration of capsule housing 1102, as described hereinabove, such that the housing exerts vibrations on the environment surrounding the capsule. Specifically, vibration of capsule housing 1102 may be intended to effect a mechanical stimulation of the wall of the gastrointestinal tract at the predetermined time of day.

In some embodiments, providing the activation input at step 1210f is responsive to a user input. In other embodiments, providing the activation input at step 1210f is automatic, and is based on control unit 1120f knowing the predetermined time(s) of day, and tracking time using clock 1126 to identify the arrival of the predetermined time(s) of day.

In some embodiments, vibration of the capsule at the predetermined time of day, which effects the mechanical stimulation, triggers the subject to have an SBM or a CSBM at a later time of day than the vibration. For example, vibration of the capsule between 12 pm and 2 pm may trigger the subject to have a CSBM between 6 pm and 8 pm.

In some embodiments, at optional step 1203f, an input may be received at the control unit, for example via a user interface thereof or via a sensor, such as the toilet bowl sensor described hereinabove or a sleep tracking sensor. For example, the input may include information pertaining to the circadian cycle of the subject, such as the subject's sleep schedule, vitality schedule, defecation schedule, and/or meal schedule. The input may be provided by the subject, medical personnel, or by a caregiver of the subject. The input may be used to set, or to adjust, the predetermined time(s) of day for the subject.

In some embodiments, the predetermined time(s) of day is preset in the control unit, or is determined at step 1200f and is provided to the control unit.

In other embodiments, at optional step 1204f, the control unit computes the predetermined time(s) of day, prior to providing the activation input(s) to the capsule. The computation of the predetermined time(s) of day may be based on user input received at step 1203f or on a location of the control unit as identified by location sensor 1124.

In some embodiments, for example when the method of FIG. 10F is used to treat an ailment of the gastrointestinal tract, a treatment session as defined in steps 1202f to 1210f may be repeatedly administered to the subject as specified in the treatment procedure for the subject, determined or obtained at step 1200f. In some embodiments, the treatment procedure includes administering a plurality of treatment sessions to the subject, substantially as described hereinabove with respect to FIG. 10F.

Reference is now made to FIG. 11, which is a schematic illustration of the circadian cycle of a person, including suitable times of day for the implementation of the methods of FIGS. 10A to 10F. The times of day shown in FIG. 11, and the corresponding body activities or characteristics at those times of day, are known in the art, and have been shown in medical and scientific research.

As will be noted, according to the typical circadian cycle shown in FIG. 11, a bowel movement is likely around 8:30 am, and bowel movements are suppressed around 10:30 pm (22:30).

As shown in the Examples below, Applicants have discovered that setting a vibrating ingestible capsule, such as the capsule described hereinabove with respect to FIGS. 9A to 9F, to vibrate during the morning hours (e.g. around 7:00-8:00 am) significantly increases the number of SBMs experienced by users in those hours.

Without wishing to be bound by theory, Applicants surmise that vibration during the morning hours, at which bowel movements are likely according to the circadian cycle, affects the walls of the gastrointestinal tract and promotes peristaltic movement, thereby assisting in completion of the bowel movement the body is likely to be promoting at that time. As shown in the Examples below, Applicants have further discovered that setting a vibrating ingestible capsule, such as the capsule described hereinabove with respect to FIGS. 9A to 9F, to vibrate during the evening hours (e.g. around 7:00-9:00 pm) significantly increases the number of SBMs experienced by users in those hours and/or in the morning hours, when the gastrointestinal system “wakes up”.

Without wishing to be bound by theory, Applicants surmise that vibration during the evening hours, prior to the body suppressing bowel movements according to the circadian cycle, affects the walls of the gastrointestinal tract and promotes peristaltic movement, thereby assisting in creating an additional bowel movement “cleaning out” the GI tract before bowel movements are suppressed, and/or that the triggered peristaltic movement which assists in generating an SBM or CSBM in the morning hours.

Without wishing to be bound by theory, the Applicants further surmise that when the gastrointestinal tract is sufficiently active with contractions suitable for generating an SBM or a CSBM, such as during the morning hours or during breakfast time, any impact of the vibrations of the capsule is small relative to the contractions already occurring. The Applicants further surmise that when the digestive system is inactive, for example not during mealtimes, vibrations of the capsule are insufficient to generate peristaltic activity of the gastrointestinal tract. However, when the digestive system is active, but typically not enough to generate a bowel movement, activation of the capsule may “assist” the gastrointestinal tract in completing a bowel movement, resulting in an increase in the number of bowel movements the user experiences during a predefined duration, e.g. per week.

Reference is now made to FIG. 12, which is a graphic illustration of the gastric pH of a person, indicating suitable times of day for the implementation of the methods of FIGS. 10A and 10F.

As seen in FIG. 12, the gastric pH of a typical person oscillates during the day, and is relatively high at typical mealtimes, and then decreases gradually until the next mealtime. Research has shown gastric pH is sensed by mechanoreceptors in the GI tract, and is tied to peristalsis in the GI tract (see for example “Acid sensing in the gastrointestinal tract” to Holtzer, (Am J Physiol Gastrointest Liver Physiol. 2007 March; 292(3): G699-G705, https://www.ncbi.nlm.nih.gov/pmc/articles/PMC4370835/) in the section about Esophago-gastro-duodenal motility).

As shown in the Examples below, Applicants have discovered that setting a vibrating ingestible capsule, such as the capsule described hereinabove with respect to FIGS. 9A to 9F, to vibrate during typical mealtimes (during the morning hours and during the evening hours) significantly increases the number of SBMs experienced by users in those hours, or in a few hours following vibration.

Without wishing to be bound by theory, Applicants surmise that vibration at mealtimes, at which there are changes in the gastric pH according to the graph provided in FIG. 12, and during which times there is likely to be peristaltic activity in the GI tract, impacts the walls of the gastrointestinal tract and supports the peristaltic movement, thereby assisting in promoting bowel movements at those times.

### EXAMPLES

Reference is now made to the following examples, which, together with the above description, illustrates the invention in a non-limiting fashion.

### Example 1

A study which included 130 participating subjects suffering from constipation was conducted. Half of the participating subjects, termed herein “trial subjects”, were treated with a vibrating gastrointestinal capsule according to a treatment protocol, in accordance with the present invention, while the other half, termed herein “sham subjects”, were treated with a sham capsule, which appeared and behaved identically to the vibrating gastrointestinal capsule prior to ingesting thereof, but did not vibrate within the subject's alimentary tract.

The treatment protocol included treatment cycles including administering one gastrointestinal capsule per day five times per week, repeated for a treatment duration of six weeks. The administered capsules included a non-chargeable battery as the power source, and a coin-type eccentric vibration motor as the vibrating agitator.

The capsules administered to the “trial subjects” were programmed to have operate in the vibration mode of operation during the morning hours following an activation time delay of at least 8 hours, and when in the vibration mode of operation, to have vibration treatment cycles including a 3 second vibration duration followed by a 16 second repose duration, for a cumulative treatment duration of 1.5 to 3 hours. During the vibration mode of operation, the force applied by the capsule housing on the surrounding environment was in the range of 200 gram-force to 500 gram-force, and the vibrational frequency was in the range of 120 Hz to 280 Hz. Different specific forces were applied to the surrounding environment, and corresponding different vibrational frequencies were attained, in different vibration cycles of the administered capsules.

Due to the activation time delay, it is assumed that vibration was affected when the capsules were disposed in a section of the large intestine of the participating subjects.

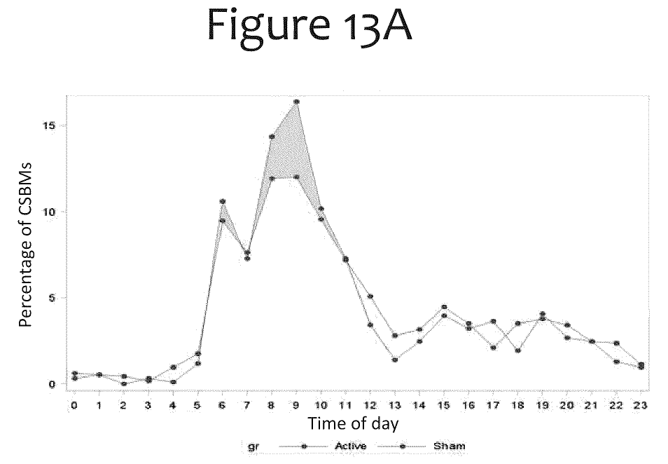

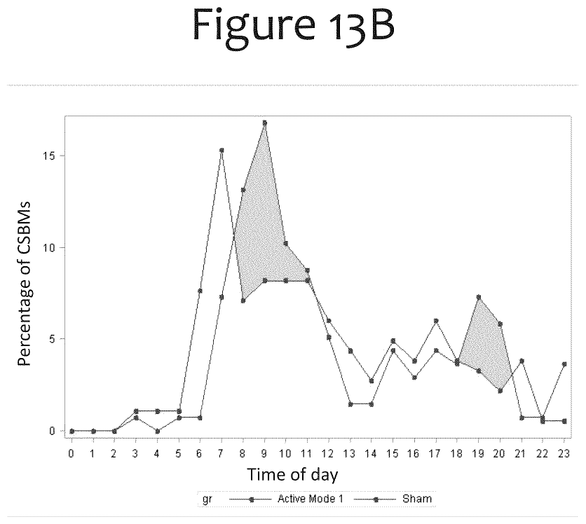

The results of the study are shown in FIG. 13A, which illustrates the percentage of complete spontaneous bowel movements (CSBMs) relative to a time from ingestion of the capsules. As seen in FIG. 13A, at the times when vibration of the capsules is effected—between 8 and 11 hours after ingestion of the capsules and during the morning hours, the subjects receiving active capsules had a significantly greater number of CSBMs than those receiving sham capsules. As such, the results illustrated in FIG. 13A indicate that vibration of the capsules in the morning hours (e.g. at times at which bowel movements are likely according to the circadian cycle), and/or coinciding with mealtimes (e.g. at times at which gastric pH is low), improves the success of the treatment—thereby providing motivation for treating subjects at specific times of day.

### Example 2

A study which included 26 participating subjects suffering from constipation was conducted. 16 of the participating subjects, termed herein “trial subjects”, were treated with a vibrating gastrointestinal capsule according to a treatment protocol, in two different arms of the study, in accordance with the present invention, while the remaining ten subjects, termed herein “sham subjects”, were treated with a sham capsule, which appeared and behaved identically to the vibrating gastrointestinal capsule prior to ingesting thereof, but did not vibrate within the subject's alimentary tract.

The treatment protocol included treatment cycles including administering one gastrointestinal capsule per day five times per week, repeated for a treatment duration of six weeks. The administered capsules included a non-chargeable battery as the power source, and a coin-type eccentric vibration motor as the vibrating agitator.

The capsules administered to the “trial subjects” were programmed to have operate in the vibration mode of operation during the early morning hours following an activation time delay of at least 8 hours, and to operate in the vibration mode of operation again during the afternoon hours. The capsules were programmed, when in the vibration mode of operation, to have vibration treatment cycles including a 3 second vibration duration followed by a 16 second repose duration, for a cumulative treatment duration of 1.5 to 3 hours. During the vibration mode of operation, the force applied by the capsule housing on the surrounding environment was in the range of 200 gram-force to 500 gram-force, and the vibrational frequency was in the range of 120 Hz to 280 Hz. Different specific forces were applied to the surrounding environment, and corresponding different vibrational frequencies were attained, in different vibration cycles of the administered capsules.

Due to the activation time delay, it is assumed that vibration was affected when the capsules were disposed in a section of the large intestine of the participating subjects.

The results of the study are shown in FIG. 13B, which illustrates the percentage of complete spontaneous bowel movements (CSBMs) relative to a time from ingestion of the capsules. As seen in FIG. 13B, at the times when vibration of the capsules is effected—between 8 and 11 am, and between 7 and 9 pm, the subjects receiving active capsules had a significantly greater number of CSBMs than those receiving sham capsules. As such, the results illustrated in FIG. 13B indicate that improvement of the subject's symptoms of the subject's symptoms by addition of CSBMs is coincidental with times at which the capsule vibrates, thus demonstrating the efficacy of the capsules, and the benefit in activating the capsules to operate at specific times of day, which coincide with mealtimes and/or at which the circadian cycle indicates that a bowel movement is likely and prior to bowel movements becoming unlikely according to the circadian cycle.

### Example 3

A study was conducted, which study included 299 participating subjects suffering from chronic idiopathic constipation according to the Rome III criteria and who have not experienced relief of their symptoms from available therapies. 155 of the participating subjects, termed herein “trial subjects”, were treated with a vibrating gastrointestinal capsule according to a treatment protocol, in accordance with the present invention, while the remaining 144 subjects, termed herein “placebo subjects”, were treated with a placebo capsule, which appeared identically to the vibrating gastrointestinal capsule prior to ingesting thereof, but disintegrated within the subject's alimentary tract.

All subjects were observed and monitored for two weeks, termed a “run-in period”. During the run-in period, the subjects were asked to refrain from taking any medication or supplement to relieve their constipation. The subjects completed a diary every day, in which the subjects reported regarding their daily bowel movements, change of diet, change of symptoms, and change in general health conditions. The run in period was used to set a baseline for the number of SBMs, and CSBMs that each subject experiences per week.

During the trial period, subjects continued to complete the diary, and to report their experience regarding daily bowel movements, as well as regarding straining and consistency of bowel movements (in accordance with the Bristol stool scale).

The treatment protocol included treatment cycles including administering one gastrointestinal capsule per day five times per week, repeated for a treatment duration of eight weeks. The administered capsules included a non-chargeable battery as the power source, and a coin-type eccentric vibration motor as the vibrating agitator.

The capsules administered to the “trial subjects” were programmed to operate in the vibration mode of operation at two predetermined times of day, for approximately two hours each time, for a total of five vibration periods.

The capsules administered to the “trial subjects” were programmed to operate in the vibration mode of operation during lunchtime and again during suppertime, for three consecutive days. As such, each capsule vibrated during lunchtime and supper time on day 1, during lunchtime and suppertime on day 2, and during lunchtime on day 3, if the capsule was still in the gastrointestinal tract of the user.

The capsules were programmed, when in the vibration mode of operation, to have vibration treatment cycles including a 3 second vibration duration followed by a 16 second repose duration, for a cumulative treatment duration of 1.5 to 3 hours. During the vibration mode of operation, the force applied by the capsule housing on the surrounding environment was in the range of 200 gram-force to 500 gram-force, and the vibrational frequency was in the range of 120 Hz to 280 Hz. Different specific forces were applied to the surrounding environment, and corresponding different vibrational frequencies were attained, in different vibration cycles of the administered capsules.

The capsules were programmed to vibrate only following an activation time delay of at least 8 hours. Due to the activation time delay, it is assumed that vibration was affected when the capsules were disposed in a section of the large intestine of the participating subjects.

The capsules were programmed to vibrate for multiple consecutive days. Additionally, capsules were administered to the subjects five days per week, i.e. on some consecutive days. Consequently, there were times at which multiple capsules simultaneously operated in the vibration mode of operation within the gastrointestinal tract of a subject, at different locations along the gastrointestinal tract.

Results of the study, with respect to number of bowel movements, are shown in Table 1.

TABLE 1

| | Trial subjects | | | Placebo | | | |
|---|---|---|---|---|---|---|---|
| Result | n | N | % | n | N | % | p-value |
| At least 1 additional | 63 | 155 | 40.65 | 33 | 144 | 22.92 | <0.0001 |
| CSBM for at least 6 out of | | | | | | | |
| 8 weeks | | | | | | | |
| At least 2 additional | 36 | 155 | 23.23 | 17 | 144 | 11.81 | 0.0008 |
| CSBM for at least 6 out of | | | | | | | |
| 8 weeks | | | | | | | |

The results of the study with respect to number of bowel movements are further shown in FIGS. 14A and 14B. FIG. 14A illustrates the per-week proportion of subjects who had one additional CSBM relative to the baseline, for at least six out of eight weeks. FIG. 14B illustrates the per-week proportion of subjects who had two additional CSBMs relative to the baseline, for at least six out of eight weeks. Stated differently, for each week in FIGS. 14A and 14B, the value represents the percentage of the subjects who met the criteria (i.e. had one or two additional CSBM per week for six out of eight weeks) and who had an additional one or two CSBM on that specific week.

As is clearly evident from the results presented herein, more than 40% of the trial subjects experienced at least one additional CSBM per week for at least six out of the eight weeks. Furthermore, more than 23% experienced at least two additional CSBMs per week for at least six out of the eight weeks. The percentage of placebo subjects to experience the same level of improvement is approximately half that of the trial subjects, demonstrating the efficacy of the capsules.

Additionally, as discussed hereinabove, in Example 2 the percentage of subjects who experienced additional CSBMs is approximately 15%, as compared to the results of Example 3 in which the percentage of trial subjects who experienced additional CSBMs is greater than 23%. This is indicative of vibration during lunchtime and suppertime being more efficacious than vibration during breakfast time and lunchtime, and supports the Applicant's understanding regarding therapeutic windows for providing vibration.

Further, as discussed hereinabove, in Example 2 each capsule vibrated twice, i.e. for a single day. As a result, no two capsules were vibrating simultaneously in different parts of the GI tract. By contrast, in Example 3, two, and in some cases even three, capsules were vibrating concomitantly in different parts of the GI tract. As such, the difference in results between Examples 2 and 3 may indicate an advantage in providing simultaneous vibration by multiple capsules in different portions of the GI tract.

Comparison of the results of Examples 2 and 3 indicates that there are one or more better times for operation of vibrating capsules according to the present invention. Such better times may be during times in which the digestive system is active, e.g. during mealtime, but not during times that the gastrointestinal tract is sufficiently active with contractions suitable for generating an SBM or CSBM.

Without wishing to be bound by theory, the Applicants surmise that when the gastrointestinal tract is sufficiently active with contractions suitable for generating an SBM or a CSBM, such as during the morning hours or during breakfast time, any impact of the vibrations of the capsule is small, or even negligible, relative to the contractions already occurring. The Applicants further surmise that when the digestive system is inactive, for example not during mealtimes, vibrations of the capsule are insufficient to generate peristaltic activity of the gastrointestinal tract. However, when the digestive system is active, but typically not active enough to generate a bowel movement, activation of the vibrating capsule may “assist” the gastrointestinal tract in completing a bowel movement, resulting in an increase in the number of bowel movements the user experiences during a predefined duration, e.g. per week.

The results of the study with respect to straining are further shown in FIG. 14C. FIG. 14C illustrates the per-week proportion of subjects who experienced an improvement in straining (i.e. reduction of straining during defecating) of at least 3 points in an 11 point scale. Stated differently, for each week in FIG. 14C, the value represents the percentage of the subjects who met the criteria (i.e. had an improvement in straining of at least 3 points in the 11 point scale) and who experienced that improvement on that specific week.

As seen in FIG. 14C, during all eight weeks of the trial, the proportion of trial subjects who experienced an improvement of at least 3 points in straining is greater than the proportion of placebo subjects who experienced the same level of improvement. As seen, in at least six of the eight weeks of the trial, more than 30% of the trial subjects experienced an improvement in straining.

The results of the study with respect to stool consistency are further shown in FIG. 14D. FIG. 14D illustrates the per-week proportion of subjects who experienced an improvement in stool consistency (i.e. an increase in the Bristol stool score of defecations) of at least 1 point in the point Bristol stool scale. Stated differently, for each week in FIG. 14D, the value represents the percentage of the subjects who met the criteria (i.e. had an increase of at least 1 point in the Bristol stool score) and who experienced that improvement on that specific week.

As seen in FIG. 14D, during all eight weeks of the trial, the proportion of trial subjects who experienced an improvement of at least 1 points in the Bristol stool score is greater than the proportion of placebo subjects who experienced the same level of improvement. As seen, seven of the eight weeks of the trial, more than 40% of the trial subjects experienced an improvement in stool consistency, and in five of the eight weeks more than 50% of the trial subjects experienced an improvement in stool consistency.

As such, it is clearly evident that in addition to improving the number of CSBMs of the subjects, the treatment protocol used in Example 3 is also successful in improving subjects' need for straining, and improving Bristol stool scores, for many of the trial subjects.

In some cases, the increase in the number of weekly bowel movements, together with improvement in the straining and/or stool consistency parameters, was sufficient to remove some subjects from the Rome III criteria or other criteria used to identify the clinical definition of chronic constipation severity, thereby not only improving these subjects' symptoms, but also improving their diagnosis.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A gastrointestinal treatment system for treatment of a disorder in a gastrointestinal tract of a subject, the system comprising:
(a) an input source providing input relating to a characteristic of a circadian cycle of the subject;
(b) a control unit, functionally associated with said input source, said control unit adapted to determine a user-specific time of day based on the input relating to the characteristic of the circadian cycle of the subject; and
(c) a gastrointestinal capsule for vibrating in the gastrointestinal tract of the subject following ingestion of said gastrointestinal capsule, said gastrointestinal capsule comprising:
a housing;
a vibrating agitator adapted such that, in a first vibrating mode of operation, said housing exerts vibrations on an environment surrounding said capsule;
a power supply disposed within said housing and adapted to power said vibrating agitator; and
a controller adapted, in response to receipt of an activation input from the control unit, to activate said vibrating agitator to operate in said first vibrating mode of operation at the user-specific time of day.

2. The gastrointestinal treatment system of claim 1, wherein the input source comprises a user interface, adapted to receive input from the subject or from a caretaker of the subject.

3. The gastrointestinal treatment system of claim 1, wherein the input source comprises a sensor, adapted to sense a characteristic relating to the circadian cycle of the subject and to provide the input.

4. The gastrointestinal treatment system of claim 1, wherein said input includes input relating to a sleep time or sleep schedule of the subject.

5. The gastrointestinal treatment system of claim 1, wherein said input includes input relating to a meal time or meal schedule of the subject.

6. The gastrointestinal treatment system of claim 1, wherein said input includes input relating to a time of defecation of the subject.

7. The gastrointestinal treatment system of claim 1, wherein said input includes input relating to a vitality schedule of the subject.

8. The gastrointestinal treatment system of claim 1, wherein said user-specific time of day is a user-specific mealtime.

9. The gastrointestinal treatment system of claim 1, further comprising a computer readable memory adapted to store a subject profile for the subject, the subject profile including subject data received as said input.

10. The gastrointestinal treatment system of claim 1, wherein said control unit is adapted to provide said user-specific time of day, to said gastrointestinal capsule, as part of said activation input.

11. The gastrointestinal treatment system of claim 1, wherein said control unit is adapted to incorporate said user-specific time of day into a vibration protocol and to program said gastrointestinal capsule with said vibration protocol.

12. A method of treating a gastrointestinal condition of a subject, the method comprising:
(a) receiving input relating to a characteristic of a circadian cycle of the subject;
(b) determining a user-specific time of day based on the input relating to the characteristic of the circadian cycle of the subject;
(c) programming a gastrointestinal capsule being adapted to transit a gastrointestinal tract of the subject, with said user-specific time of day, said gastrointestinal capsule having:
(1) a housing;
(2) a vibrating agitator, the vibrating agitation mechanism adapted such that, in a first vibrating mode of operation, said housing exerts vibrations on an environment surrounding said gastrointestinal capsule;
(3) a power supply disposed within said housing and adapted to power said vibrating agitator; and
(4) a controller adapted to activate said vibrating agitator to operate in said first vibrating mode of operation;
(d) following ingestion of said gastrointestinal capsule by the subject, and when said gastrointestinal capsule is in an operative state, activating said vibrating agitator to operate in said first vibrating mode of operation said user-specific time of day.

13. The method of claim 12, wherein said receiving input comprises receiving input relating to a sleep schedule of the subject.

14. The method of claim 12, wherein said receiving input comprises receiving input relating to a meal schedule of the subject.

15. The method of claim 12, wherein said receiving input comprises receiving input relating to a time of defecation of the subject.

16. The method of claim 12, wherein said receiving input comprises receiving input relating to a vitality schedule of the subject.

17. The method of claim 12, wherein said receiving input comprises receiving said input from the subject or from a caregiver of the subject.

18. The method of claim 12, wherein said receiving input comprises receiving said input from a sensor.

19. The method of claim 12, further comprising, storing said input in a subject-profile associated with the subject.

20. The method of claim 12, wherein said programming comprises providing the user-specific time of day to said gastrointestinal capsule as part of an activation input.
